(19) United States
(12) Patent Application Publication
(10) Pub. No.: US 2025/0316385 A1
(43) Pub. Date: Oct. 9, 2025

(54) SCLCPHENO-SEQ, A TARGETED CAPTURE PANEL AND ASSOCIATED METHODOLOGY TO CALL THE ACTIVITY OF KEY TRANSCRIPTION FACTORS OF CLINICAL RELEVANCE TO SMALL CELL LUNG CANCER FROM PATIENT LIQUID BIOPSIES

(71) Applicant: Fred Hutchinson Cancer Center, Seattle, WA (US)

(72) Inventors: David MacPherson, Seattle, WA (US); Joseph Hiatt, Seattle, WA (US); Gavin Ha, Seattle, WA (US); Anna-Lisa Doebley, Seattle, WA (US)

(73) Assignee: Fred Hutchinson Cancer Center, Seattle, WA (US)

(21) Appl. No.: 19/170,035

(22) Filed: Apr. 3, 2025

Related U.S. Application Data
(60) Provisional application No. 63/573,699, filed on Apr. 3, 2024.

(51) Int. Cl. G16H 50/20; G16B 20/30

(52) U.S. Cl. CPC G16H 50/20; G16B 20/30

(57) ABSTRACT

Small cell lung cancer (SCLC) exhibits distinct molecular subtypes characterized by activation of transcription factors (TFs) such as ASCL1, NEUROD1, POU2F3, and REST, but clinical translation has been limited by tissue scarcity. Here, a cell-free DNA (cfDNA) targeted sequencing assay is disclosed that analyzes DNA fragmentation patterns to infer nucleosome profiles at TF binding sites and gene transcription start sites (TSSs) and also detects exonic mutations in certain genes. Application to plasma cfDNA from SCLC patient-derived xenograft models faithfully captured signatures of TF activity and gene expression and revealed a subset of highly informative nucleosome profiling loci including TSSs of key genes including ATOH1, POU2AF2, and targets of SCLC subtype defining TFs. Prediction models of ASCL1, NEUROD1, and REST activity achieved AUCs (0.82-1.00) in SCLC patient samples while a predictor of SCLC vs NSCLC histology achieved an AUC of 0.99. Targeted cfDNA nucleosome profiling can enable SCLC subtyping to improve patient care.

### CROSS-REFERENCES TO RELATED APPLICATIONS

This application claims the benefit of U.S. Provisional Application No. 63/573,699, filed Apr. 3, 2024, the disclosure of which is incorporated herein in its entirety.

### STATEMENT OF GOVERNMENT LICENSE RIGHTS

This invention was made with Government support under CA228944 awarded by the National Institutes of Health. The Government has certain rights in the invention.

### BACKGROUND

The present disclosure uses targeted sequencing to identify previously unrecognized heterogeneity in cfDNA nucleosome profiling signals, resulting in a practical method for non-invasive SCLC genotype and phenotype assessment. This framework also has immediate application to assay NSCLC-SCLC trans-differentiation and can be generalized to study and monitor many other cancer types.

Small cell lung cancer (SCLC) is an aggressive neuroendocrine (NE) neoplasm with poor clinical outcomes and a paucity of therapeutic advances despite decades of concerted effort (1). Treatment naïve disease is typically sensitive to platinum doublet chemotherapy but the development of recurrent disease that is chemotherapy resistant is essentially ubiquitous and often rapid (1). SCLC is typically diagnosed using fine needle aspiration of mediastinal lymph nodes which may contain large necrotic regions and is rarely resected (1). As such, available tissue specimens are often inadequate for detailed immunophenotyping and deep genomic profiling, impeding efforts to understand SCLC biology and link biological features of SCLC to treatment responses in clinical trials. Methods to non-invasively profile SCLC tumors are therefore needed to advance care for patients with SCLC.

Tissue sampling is also a key obstacle to optimal care for patients with non-small cell lung cancer (NSCLC). For example, patients with NSCLC harboring driver mutations often experience prolonged responses to targeted inhibitors; however, a key mechanism of acquired resistance is trans-differentiation to SCLC, with implications for subsequent treatment selection (2, 3). Detecting this event currently requires a biopsy of a progressing lesion for pathologic evaluation, but repeat sampling is often infeasible or undesirable. Non-invasive assessment of lung cancer histology could reduce or eliminate the need for tissue biopsy to identify trans-differentiation to SCLC and substantially improve care for patients with driver mutation positive NSCLC.

Despite tissue scarcity, there has been major recent progress in understanding the genetic and molecular phenotypic landscape of SCLC. Genetically, SCLC is characterized by nearly ubiquitous biallelic inactivating mutations of TP53 and highly frequent biallelic mutation of RB1 (4-6). Other recurrent genomic abnormalities have also been described including loss of function mutations in various chromatin modifiers and focal amplifications of MYC, MYCL and MYCN (6-8). SCLC tumors very rarely harbor activating mutations in oncogenic drivers with available targeted therapies, and efforts to therapeutically target specific genetic subsets of SCLC remain exploratory (1). Nevertheless, SCLC genetics remains relatively poorly characterized compared to other common tumor types, particularly in extensive stage samples and following therapy resistance, and it remains critical to identify genetic drivers in SCLC and link mutations to therapy response even when biopsies are unavailable.

Considerable effort has also been devoted to understanding gene expression and inter-tumoral heterogeneity in SCLC, with multiple promising emerging insights. First, SCLC tumors tend to highly express one of the master regulatory transcription factors (TFs) ASCL1, NEUROD1 or POU2F3 and preclinical models with differential activity of these TFs exhibit intriguing biological differences, including therapeutic sensitivities (9-12). Based on these findings, a taxonomy of SCLC “transcriptional subtypes” defined by TF activity has been proposed (10). ATOH1, a TF active in certain neurosensory cells, has also been proposed to identify a subtype of SCLC (13). Second, although long appreciated (14, 15), variation in the degree of NE differentiation in SCLC has recently been explored more carefully (16-18). A neuroendocrine-low subset of SCLC has also been identified to express REST. REST is a repressor of neuronal cell fate that is variably active in SCLC, with REST-active SCLC exhibiting a suppressed NE gene expression profile (11, 19, 20).

Additionally, recent large-scale IHC analyses have established that ASCL1 and NEUROD1 are co-expressed in a substantial fraction of tumors (21-24), and a double-positive (ASCL1+NEUROD1) subtype has also been proposed based on bulk transcriptomic analysis of SCLC patient derived xenograft (PDX) models (25), although thresholds for sample classification have yet to be defined. In addition, regulation of other key genes, including the MYC family, the NOTCH pathway, and cell surface markers such as DLL3 and SEZ6 that are targets of antibody-drug conjugates or BiTEs make up additional phenotypes that could inform clinical decisions in SCLC.

Critically, pioneering studies have linked both TF activity-defined SCLC subtype and relative NE status with patient outcomes (11, 26). For example, in an analysis of patients receiving chemotherapy with or without an immune checkpoint inhibitor (ICI) in IMpower133, Gay et al. identified an “inflamed” subset of tumors that were ASCL1, NEUROD1, and POU2F3-low, and which derived the most benefit from ICI (11). There is also considerable interest in inhibiting LSD1 as a treatment approach for SCLC. It has also been shown in immune deficient models that the ability of LSD1 inhibition to activate NOTCH and suppress ASCL1 was linked to the strength of response (14). Also, NE-low (i.e., REST-high) SCLC tends to express MHC-I, while ASCL1-active SCLC tends to exhibit suppression of MHC-I15, suggesting that transcriptional subtypes of SCLC might differentially respond to LSD1 inhibitor in combination with immune checkpoint inhibitors. On the other hand, although POU2F3-positive tumors are also REST-high and low for NE markers, these patients did not appear to benefit from ICH and had poor outcomes overall (11). In a separate study, patients who experienced clinical benefit from second line ICI were more likely to harbor REST-high, NE-low tumors (26). Other investigational drug targets are also correlated with one or more of these characteristics; for example, the Notch ligand DLL3, which is the target of multiple investigational therapeutic strategies, is correlated with ASCL1 and anti-correlated with REST (12). Multiplexed assessment of TF activity is therefore of considerable importance to SCLC clinical research efforts but is technically challenging due to tissue scarcity. Further, high expression and activity of key transcriptional regulators define diverse molecular subtypes of SCLC, and the inability to link biologically distinct subsets to clinical responses exemplifies the major limitations of current treatment strategies and poor outcomes for SCLC.

Further, trans-differentiation from non-small cell lung cancer (NSCLC) into SCLC is a resistance mechanism to targeted therapies. Tissue sampling is also a key obstacle to optimal care for patients with NSCLC. For example, patients with NSCLC harboring driver mutations often experience prolonged responses to targeted therapies such as EGFR tyrosine inhibitors; however, a key mechanism of acquired resistance is transformation to SCLC. Identification of SCLC transformation can have implications for subsequent treatment selection, including a strong response to platinum-etoposide chemotherapy (a typical treatment for SCLC). Patients with EGFR/TP53/RB1-mutant tumors are particularly at risk. Transformation from NSCLC to SCLC also occurs in EGFR wild-type NSCLC, for example, in patients with ALK alterations treated with ALK tyrosine kinase inhibitors and with ROS1 rearranged NSCLC treated with crizotinib. Transformation events are under-diagnosed because repeat biopsies of progressing lesions for pathologic evaluation are often not performed. Moreover, a single biopsy may not be representative of the histological heterogeneity following SCLC-transformation between the metastatic sites. Also, further phenotyping of transformed SCLC into different subsets based on activation of master regulatory transcription factors may inform our understanding of ways to best treat transformed SCLC patients. Thus, non-invasive assessment of lung cancer histology can be employed in the clinic to identify transformation to SCLC and quantify phenotypic heterogeneity in order to substantially improve care for patients with driver-mutation-positive NSCLC.

Circulating tumor DNA (ctDNA) provides a non-invasive window to study transcriptional regulation and to classify SCLC tumor phenotypes and to use the results to substantially improve patient care. Cell-free DNA (cfDNA) is released into circulation by dying cells (27); in patients with cancer, a variable portion of cfDNA is derived from tumor cells. Sequencing cfDNA to non-invasively detect tumor-specific mutations is becoming a mainstay of routine clinical care (28), while pioneering recent studies have analyzed the fragmentation pattern of cfDNA to infer the position of nucleosomes in cells-of-origin (29-31). Because nucleosome positioning is strongly influenced by transcription factors and other proteins involved in gene expression regulation, TF activity and gene expression can be inferred directly from cfDNA coverage patterns. This strategy, referred to as cfDNA “nucleosome profiling,” has been adapted to multiple important applications including cancer detection (32) and subtyping of breast and prostate cancer (33, 34), but has not been previously applied to distinguish subtypes of SCLC.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides a method of determining a lung cancer type from a sample comprising cell-free DNA isolated from a patient biological sample, the method comprising: obtaining nucleotide sequence read data generated from the sample comprising cell-free DNA; performing a computer-implemented method, the method comprising: receiving, by a computing system, sequence read data, wherein the sequence read data includes a plurality of fragment reads, wherein each fragment read has a fragment length and a GC content indicating a percentage of bases in the fragment read that are G or C; determining, by the computing system, GC bias values for each fragment read based on the fragment length and the GC content of the fragment read; generating, by the computing system, a genomic coverage distribution that is adjusted for GC bias using the sequence read data and the GC bias values; predicting, by the computing system, the cell type based on the genomic coverage distribution; and determining the lung cancer type based on the prediction provided by the computer system.

The determination of the GC bias value can be based on the fragment length and the GC content of the fragment read and includes: counting a number of observed reads of each combination of fragment length and GC content to determine GC counts for the sequence read data; dividing the GC counts by corresponding GC frequencies in a GC frequency matrix to determine a GC bias for each fragment length; normalizing a mean GC bias for each fragment length to determine rough GC bias values; and smoothing the rough GC bias values to determine the GC bias values.

In one embodiment of the method subsequent to determination of the GC bias, the lung cancer is determined to be small cell lung cancer (SCLC) or non-small cell lung cancer (NSCLC). In another embodiment the SCLC phenotype determination includes determining expression of one or more genes of interest.

In a certain embodiment the method is performed a plurality of time over time, and wherein the method further comprises detecting a change from NSCLC to SCLC over time. The method can be used wherein the patient receives a cancer therapy between performances of the method, and wherein the method further comprises determining the responsivity of the NSCLC or SCLC to the treatment.

The method can encompass a step wherein the sequence read data is generated from a panel of genomic targets. In certain embodiments, the panel of genomic targets can comprise transcription factor binding sites (TFBSs) of one or more transcription factors associated with SCLC. In specific embodiments of the method, the one or more transcription factors associated with SCLC comprise one or more of ASLC, ATOH1, NEUROD1, POU2F3, REST, and wherein the method comprises determining the nucleosome occupancy of the TFBSs.

In certain embodiments of the method the TFBSs are identified by ChIP-seq data, and the TFBSs are retained in the panel if they are proximal to a transcription start site of a gene associated with lung cancer. In certain embodiments of the method, the panel of genomic targets comprise transcription start sites (TSSs) for one or more markers associated with lung cancer, wherein the method comprises determining the nucleosome occupancy of the TSSs.

The method can further comprises administering an effective treatment to the patient based on the determined cancer subtype. In specific embodiments, the method further comprises administering an effective treatment to the patient based on the transition of the lung cancer from NSCLC to SCLC.

The present disclosure also discloses a method for treating a patient with lung cancer comprising: obtaining nucleotide sequence read data generated from the sample comprising cell-free DNA; performing a computer-implemented method comprising; receiving, by a computer system receiving, by a computing system, sequence read data, wherein the sequence read data includes a plurality of fragment reads, wherein each fragment read has a fragment length and a GC content indicating a percentage of bases in the fragment read that are G or C; determining, by the computing system, GC bias values for each fragment read based on the fragment length and the GC content of the fragment read; generating, by the computing system, a genomic coverage distribution that is adjusted for GC bias using the sequence read data and the GC bias values; and predicting, by the computing system, the cell type based on the genomic coverage distribution. Based on the prediction of the cell type, the lung cancer type is based on the prediction provided by the computer system; and an effective therapy for the lung cancer type detected is administered to the patient.

In certain embodiments, the comprises determining the GC bias value based on the fragment length and the GC content of the fragment read includes: counting a number of observed reads of each combination of fragment length and GC content to determine GC counts for the sequence read data; dividing the GC counts by corresponding GC frequencies in a GC frequency matrix to determine a GC bias for each fragment length; normalizing a mean GC bias for each fragment length to determine rough GC bias values; and smoothing the rough GC bias values to determine the GC bias values.

In certain embodiments, the method determines that lung cancer is small cell lung cancer (SCLC) or non-small cell lung cancer (NSCLC). In other embodiments, the method determines the SCLC phenotype and the method includes determining expression of one or more genes of interest.

In certain embodiments, the method is performed a plurality of times over time, and the method can further comprise detecting a change from NSCLC to SCLC over time.

In certain embodiments, the method comprises generating sequence read data from a panel of genomic targets. In more specific embodiments of the method, the panel of genomic targets comprises transcription factor binding sites (TFBSs) of one or more transcription factors associated with SCLC and/or transcription start sites (TSSs) for one or more markers associated with lung cancer.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings, wherein:

FIGS. 1A to 1E show an integrated cfDNA sequencing assay for mutation detection and inference of tumor TF activity and gene expression. FIG. 1A is an illustration of targeted sequencing panel design (panel 1), sample characteristics (panel 2), and analysis strategy (panels 3-4). (1) Coding regions of cancer-related genes, transcription start sites (TSSs) and transcription factor binding sites (TFBSs) of interest were targeted by a custom panel. At TSSs and TFBSs, nucleosomes (grey) were arranged in a more organized fashion around active sites, resulting in an excess of nucleosome-protected DNA fragments (orange) and reduced DNA fragments from open chromatin. DNA was enriched with probes covering 360 bp around TSSs and 1000 bp around TFBSs. (2) Plasma samples, with or without matched tumor tissue and buffy coat genomic DNA, were collected from patients and immunocompromised mice harboring lung cancer PDX models. (3) Sequencing reads were aligned to human and mouse genomes to remove signal from non-tumor cells in PDX samples. (4) The Griffin normalization and bias correction algorithm was applied to generate targeted nucleosome profiles for subsequent analysis and interpretation. SCLC, small cell lung cancer; TF, transcription factor; TFBS, transcription factor binding site; TSS, transcription start site; Mb, megabase; ChIPseq, chromatin immunoprecipitation sequencing; NSG, NOD/SCID/gamma; PDX, patient derived xenograft; IHC, immunohistochemistry. FIG. 1B shows a number of plasma samples obtained, filtered and analyzed. * indicates that three samples grouped into the PDX category were POU2F3-positive cell lines. Samples were excluded (excl.) due to quality control (QC) parameters of inadequate sequencing coverage or visually irregular data (n=1), or low tumor fraction (<5%). Excl., excluded; NSCLC, non-small cell lung cancer; PBMC, peripheral blood mononuclear cell; pts, patients. FIG. 1C shows tumor fraction in cfDNA predicted by ichorCNA for non-malignant (n=4), LUAD (n=5), LUSC (n=4), and SCLC (n=70) patient samples that passed filters. Distribution kernel density estimates are shown as violins. LUAD, lung adenocarcinoma; LUSC; lung squamous cell carcinoma. FIG. 1D shows tumor fraction (top row) and detected mutations from cfDNA in SCLC patients with available matched buffy coat sample (n=39) plotted with CoMut. The genes shown were mutated in at least 6 samples or of special interest in SCLC. FIG. 1E shows characteristics of cfDNA libraries and tissue phenotype from PDX and patient samples with matched tissue. Human fraction (fraction of reads preferentially aligned to human vs mouse) for PDX samples and tumor fraction for patient samples is shown on top row. Heatmap of RNAseq expression or IHC measurement of gene/protein level is shown in second row. REST activity as determined by reduced expression of REST target genes is shown in third row. Tumor subtype for NSCLC samples (LUAD, LUSC, or LCNEC) and SCLC samples, which are assigned into a TF status-based group based on gene expression, is shown in fourth row. Non-malign., non-malignant.

FIGS. 2A to 2E show tumor cell TF activity and gene expression signatures were apparent in targeted cfDNA nucleosome profiling data. FIG. 2A is an illustration of a calculation of a composite nucleosome profile for a feature of interest (TFBSs or TSSs) by averaging the Griffin nucleosome profile of individual sites within a sample or group of samples, followed by display as a curve or heatmap. Nuc. prof., nucleosome profile. FIG. 2B is composite nucleosome profiles from cfDNA targeted sequencing at ASCL1, NEUROD1, POU2F3, and REST sites in neuroendocrine PDX samples (n=20), colored by sample TF activity. Samples with positive TF activity (light gray (yellow)) have reduced central coverage and more variable composite nucleosome profiles. FIG. 2C depicts unsupervised hierarchical clustering of neuroendocrine PDX samples (n=20) by composite nucleosome profiles, shown alongside tissue characteristics and difference in tissue TF expression in groups of samples defined by first bifurcation of dendrogram. First column: Dendrogram and heatmap of clustered composite nucleosome profiles, where dendrogram branches are colored by two groups that result from clustering. Second column: Tissue transcript level by RNAseq. Third column: Binary label of sample TF activity as determined by tissue RNAseq. Fourth column: Kernel density estimates of tissue transcript level in groups of samples defined by first bifurcation of dendrogram shown as violin with median shown as horizontal bar and p value (two sided Mann-Whitney U test), and groups are labeled as “Low” or “High” by relative magnitude of median TF transcript level. Color of violins corresponds to dendrogram branches. Unsupervised clustering results in sample groups with significantly different TF expression for NEUROD1, POU2F3, and REST. Act., activity; exp., expression; clust., cluster. FIG. 2D depicts composite nucleosome profiles from cfDNA targeted sequencing of TSSs (n=13,240) in neuroendocrine PDX models (n=20) calculated by aggregating nucleosome profiles from all samples, binning TSSs into deciles by transcript level of associated genes, and computing composite (average) nucleosome profile for each decile. As gene expression increased, the composite nucleosome profiles showed a progressively more pronounced gain in coverage downstream of the TSS and a loss of coverage at and upstream of the TSS. FIG. 2E depicts distribution of TSS amplitude values at individual TSSs, calculated as the difference in relative coverage at +120 bp and −45 bp from the TSS, for individual TSS by gene expression decile (n=13,240 TSSs in 20 neuroendocrine PDX models), shown with Pearson's r and p value. TSS amplitude was correlated with gene expression although distributions were widely overlapping across expression deciles.

FIGS. 3A to 3H show the analysis of selected highly informative sites preserved strong signals of TF activity and gene expression. FIG. 3A provides an illustration of the strategy to determine informativeness of individual TFBS and TSSs in targeted panel. In the first step, cfDNA nucleosome profiles at each individual site (n=1535 TFBSs and n=5274 TSSs of variably expressed genes) in lung cancer PDX samples (n=25) were subjected to k-means clustering with k=2. In the second step, at each TFBS, sample cluster assignments were compared with known sample TF activity using the adjusted Rand index; at each TSS, sample cluster assignments were compared with known tissue gene expression of the gene corresponding to that TSS using a two-sided Mann-Whitney U test (TSSs). TFBSs with higher adjusted Rand index and TSSs with lower Mann-Whitney U test p values were considered more informative. FIG. 3B depicts the distribution of TFBS individual site adjusted Rand index values; annotation denotes number of sites with adjusted Rand index≥0.5. FIG. 3C depicts the distribution of TSS individual site analysis two-sided Mann Whitney U test p values; annotation denotes number of sites with p<0.01.

FIG. 3D depicts average nucleosome profiles (+/−standard deviation), based on sample groups formed by k-means clustering at each site, at four individual ASCL1 TFBS with highest adjusted Rand index, in samples in high ASCL1 expression group (top panel) and low ASCL1 expression group (bottom panel). Legend reports genomic location of site, adjusted Rand index (Rand idx.), and number of samples in higher and lower TF expression cluster. Average nucleosome profiles at informative TFBSs (i.e., with higher adjusted Rand index) appear to adopt site-specific characteristic profile shapes associated with high or low TF activity. FIG. 3E depicts average nucleosome profiles (+/−standard deviation), based on sample groups formed by k-means clustering, at five individual TSSs with smallest Mann Whitney U test p value in samples in group with higher expression of corresponding gene (top panel) and lower expression of corresponding gene (bottom panel). Legend reports gene symbol, Mann Whitney U test p value of gene expression difference between clusters (based on tissue RNAseq), mean gene expression level in each cluster [log₂(TPM+1)], and number of samples in each cluster. Average nucleosome profiles at informative TSSs (i.e., with small Mann-Whitney U test p values) appear to adopt site specific characteristic profile shapes associated with high or low gene expression. FIG. 3F provides an illustration of a calculation of the profile shape similarity for an observed cfDNA nucleosome profile in a single sample at an individual TFBS or TSS. For each informative TFBS and TSS, two average nucleosome profiles (yellow) and dark gray dotted lines) are calculated from the site-specific nucleosome profiles of PDX samples that were clustered together by k=2 k-means clustering. Next, for an individual sample site-specific profile, the Euclidean distance to each of the average profiles is calculated. In parallel, each cluster (n=2) is labeled as “higher” or “lower” for expression of the TF or gene of interest based on mean TF or gene expression from tissue RNAseq. Finally, the profile shape similarity is calculated as the log₂ ratio of the distance values, where the distance to the average profile of the cluster that more lowly expresses the TF or gene of interest is in the numerator. FIG. 3G depicts cfDNA nucleosome profile shape similarity versus tissue transcript level at selected highly informative TSSs in neuroendocrine PDX samples (n=20) color coded by tissue SCLC subtype. TSSs are grouped into columns by correlation with individual SCLC TFs in independent SCLC cell line RNAseq data. Pearson r correlation of Euclidean distance ratio and tissue gene expression shown as inset. cfDNA nucleosome profile Euclidean distance ratio is tightly correlated with tissue gene expression. FIG. 3H depicts hierarchical clustering of cfDNA nucleosome profile shape similarity values at selected highly informative TSSs (n=16) in neuroendocrine PDX samples (n=20). First column: Dendrograms and heatmap of clustered TSS Euclidean distance ratios. Second column: Pearson correlations between transcript level of gene of interest (y axis) and key SCLC TFs (x axis) in independent SCLC cell line RNAseq data. Bottom row: Heatmap showing tissue transcript levels for key TFs in PDX samples. Unsupervised clustering of selected highly informative TSSs groups samples by tissue TF expression and groups genes by correlation with key SCLC TFs.

FIGS. 4A to 4H show histology predictions in patient samples using targeted cfDNA nucleosome profiling. FIG. 4A Illustrates a probabilistic model constructed to estimate cfDNA composition with respect to tumor fraction and NSCLC (LUAD or LUSC) vs SCLC. cfDNA nucleosome profiles were modeled as a linear combination of the observed coverage in tumor-free cfDNA and tumor-only cfDNA from SCLC and NSCLC PDX. This model was then applied to each sample to estimate the tumor fraction (α) and the relative fraction of SCLC to NSCLC (s, “histology score”) in cfDNA that maximized the likelihood of the observed cfDNA nucleosome profiles. An example is shown where the maximum likelihood corresponds to a tumor fraction of 0.6 (60%) and a histology score of 1, suggesting SCLC histology. FIG. 4B shows cfDNA and tumor characteristics for PDX models used in training (n=24), a PDX model which was held out (MSK_LX68), and patient samples (n=79) used in testing of a histology classification model based on highly informative TFBS and TSS nucleosome profiling data. First column: Histology score prediction with dotted line designating cutoff used to call NSCLC or SCLC. Second column: REST activity status from tissue RNAseq data (when available). Third column: Ground truth histology. Fourth column: Tumor fraction in cfDNA predicted by histology classifier. Fifth column: Tumor fraction in cfDNA predicted by ichorCNA. FIG. 4C shows Tumor fraction, and, FIG. 4D shows histology score predictions made by histology classifier in training data of non-malignant patient samples (n=4) and PDX models (n=24), based on the leave one out method. Shown as violins are kernel density estimate of distributions. P values from two-sided Mann-Whitney U test. FIG. 4E shows receiver operator characteristic (ROC) curves of histology classification in training data of non-malignant patient samples (n=4) and PDX models (n=24), based on leave-one-out method. AUC, area under the curve. FIG. 4F shows tumor fraction, and FIG. 4G shows histology score predictions made by histology classifier in test data of NSCLC (n=9) and SCLC patient samples (n=70) with ichorCNA tumor fraction prediction≥0.05. Shown as violins are kernel density estimate of distributions. P values from two-sided Mann-Whitney U test. FIG. 4H shows receiver operator characteristic (ROC) curves of histology classification in test data of NSCLC (n=9) and SCLC patient samples (n=70) with ichorCNA tumor fraction prediction≥0.05, and NSCLC (n=13) and SCLC patient samples (n=14) with ichorCNA tumor fraction prediction<0.05, AUC, area under the curve; TFx, tumor fraction.

FIGS. 5A to 5C depict SCLC subtype inference in patient samples using TF activity prediction. FIG. 5A shows cfDNA and tumor characteristics for PDX models (n=25), SCLC patient samples with matched tissue (n=24), and NSCLC patient samples (n=9). Samples are grouped into columns by PDX/patient and SCLC or not SCLC. Top row: Dendrogram and heatmap of hierarchically clustered cfDNA nucleosome profile TF scores predicted by TF score models. Second row: Ground truth/predicted REST activity shown for neuroendocrine samples. Third row: Ground truth/predicted SCLC subtype shown for neuroendocrine samples. Fourth row: Ground truth/predicted histology from histology classifier (FIG. 4B) is shown for reference. Fifth row: Tissue transcript levels (RNAseq) or protein levels (IHC, shown in black lined boxes), where available. Unsupervised clustering of TF score estimates groups samples by ground truth subtype and tissue TF expression. FIG. 5B shows distributions of cfDNA nucleosome profile TF score estimates in neuroendocrine PDX (top row) and SCLC patient (bottom row) samples by ground truth subtype (boxes show median and first and third quartiles, whiskers extend to 1.5*IQR) for ASCL1, NEUROD1, ATOH1, POU2F3, and REST (columns). Two-sided Mann Whitney U test comparing samples with positive versus negative TF status. cfDNA nucleosome profile TF scores are significantly higher in TF positive samples for ASCL1, NEUROD1, POU2F3 (no positive patient samples), and REST. FIG. 5C depicts receiver operator characteristic curves for sample TF status classification with cfDNA nucleosome profile TF score for neuroendocrine PDX (left) and SCLC patient (right) samples. AUC, area under curve.

FIGS. 6A to 6C show tumor fraction and coverage statistics for all sequenced samples. FIG. 6A shows distribution of tumor fraction estimates by ichorCNA by histology for all NSCLC/SCLC patient cfDNA samples. Dotted line denotes cutoff of 0.05 that is used to exclude low tumor fraction samples. LUAD, lung adenocarcinoma; LUSC, lung squamous cell carcinoma. FIG. 6B depicts the fold-coverage of a targeted region by histology for all PDX (left) and patient (right) cfDNA samples. Kernel density estimates of the distributions are shown as violins. LCNEC, large cell neuroendocrine carcinoma. FIG. 6C depicts the fraction of targeted region that is covered at >50× by histology for all PDX (left) and patient (right) cfDNA samples. Kernel density estimates of the distributions are shown as violins. Dotted line designates cutoff of 0.5 that is used to exclude low coverage samples.

FIG. 7A to 7C show the determination of SCLC subtype label and REST activity based on tissue RNAseq. FIG. 7A is a schematic of TF activity label determination for samples with RNAseq data. Transcript levels are considered in units of log₂(TPM+1). Samples with all TF transcripts<5 are labeled “pan-low.” Samples with POU2F3 transcript≥5 are labeled as POU2F3. The remaining samples were examined for ASCL1, NEUROD1, and ATOH1 expression. Those with ATOH1 transcript≥5 are labeled ATOH1. In parallel, because ASCL1 and NEUROD1 can be co-expressed, those with ASCL1 and/or NEUROD1≥5 were subjected to unsupervised hierarchical clustering, and samples are labeled based on relative expression of ASCL1 and NEUROD1 in the resulting clusters. FIG. 7B depicts hierarchical clustering of tissue ASCL1/NEUROD1-related gene transcript levels in PDX (left, n=506 genes) and patient (right, n=501 genes) samples with increased ASCL1 and/or NEUROD1 transcript [log₂(TPM+1)>5]. ASCL1 and NEUROD1 transcript levels are shown on the right border of heatmaps for reference. Two clear groups are formed, each characterized by dominant expression of ASCL1 or NEUROD1, and samples were considered positive for ASCL1 or NEUROD1 depending on group membership. FIG. 7C depicts hierarchical clustering of REST target gene tissue RNAseq transcript levels in PDX (left, n=334 genes) and patient (right, n=354 genes) samples. Also shown to the left of the main heatmaps are Z scores of transcript levels of REST and the REST targets CHGA, CHGB, and SYP. In PDX models, a REST-positive group (including LUAD and LUSC samples) was readily apparent. In patient samples, two samples are clustered together that have the highest REST transcript levels and the lowest levels of REST targets CHGA and CHGB and were therefore considered REST-positive.

FIG. 8 shows that the re-centering of individual TFBS windows on a consensus TF binding motif improves signal at REST sites. Neuroendocrine PDX composite cfDNA nucleosome profiles based on averaging all sites for a given TF (first column), only sites containing a consensus binding motif (second column), only sites containing a consensus binding motif after re centering the signal at individual sites to the midpoint of the consensus motif (third column), or sites without a consensus binding motif (fourth column). Re-centering individual TFBS windows visually improved the signal of REST activity but did not obviously affect the ASCL1 or NEUROD1 signal.

FIGS. 9A to 9C show that targeted nucleosome profiling closely recapitulated the signals observed in deep whole genome sequencing. FIG. 9A depicts composite nucleosome profiles by TF and sample in targeted nucleosome profiling and deep whole genome sequencing of cfDNA from the same plasma samples. FIG. 9B depicts the distribution (median, IQR, and 1.5*IQR) of individual TFBS nucleosome profile Pearson correlations between targeted nucleosome profiling and deep WGS, by TF (panel) and sample (x axis). FIG. 9C depicts the distribution (median, IQR, and 1.5*IQR) of individual TSS nucleosome profile Pearson correlations between targeted nucleosome profiling and deep WGS, by sample (x axis).

FIGS. 10A to 10C depict the central amplitude and composite nucleosome profiles of TFBS in PDX and patient samples. FIG. 10A shows the central amplitude, calculated from cfDNA composite nucleosome profiles as the difference between coverage on either side of the TFBS and coverage at the TFBS, versus tissue TF expression in neuroendocrine PDX samples (n=20), with Pearson r correlation and p value. FIG. 10B shows composite nucleosome profiles from cfDNA targeted sequencing at ASCL1 (n=448), NEUROD1 (n=330), POU2F3 (n=471), and REST (n=268) sites in SCLC patient samples with tissue RNAseq data (n=19), colored by TF expression/activity. FIG. 10C shows central amplitude, calculated from cfDNA composite nucleosome profiles as the difference between coverage on either side of the TFBS and coverage at the TFBS, versus tissue TF expression in SCLC patient samples with tissue RNAseq data (n=19), with Pearson r correlation and p value.

FIG. 11 shows the distribution of TSS fragment length entropy for individual TSS by gene expression decile. Distributions of cfDNA TSS fragment length entropy aggregated from 20 neuroendocrine PDX samples (n=13,240 per sample) and binned by gene expression decile, shown as violins. TSS fragment length entropy was correlated with gene expression (Pearson r shown as inset) although distributions were widely overlapping across expression declines.

FIG. 12 shows average TFBS nucleosome profiles based on sample groups formed by k-means clustering at NEUROD1, POU2F3, and REST sites. cfDNA average nucleosome profile (+/−standard deviation) at four individual TFBS with highest adjusted Rand index in samples in high TF expression group (top panel) and low TF expression group (bottom panel), shown for NEUROD1, POU2F3, and REST. Sample groups were determined by k-means clustering of single sample nucleosome profiles at each site. Legend reports genomic location of site, adjusted Rand index (Rand idx.), and number of samples in higher and lower TF expression cluster. Average nucleosome profiles at informative TFBSs (I, with higher adjusted Rand index) appear to adopt site-specific characteristic profile shapes associated with high or low TF activity.

FIGS. 13A and 13B depict the selection of informative sites in PDX samples was validated in patient samples. FIG. 13A shows per-sample mean of cfDNA TFBS profile shape similarity metric calculated for PDX samples (top row) and patient samples with tissue RNAseq data (bottom row) and for all sites or only sites with adjusted Rand index≥0.5 (i.e., highly informative sites), grouped by whether a sample is TF-negative (dark gray) or TF positive (light gray), with two sided Mann-Whitney U test p value, and difference in median of sample means. Selection of informative sites in PDX samples leads to an increase in the difference of median of sample means of the profile shape similarity metric in patient samples, supporting the generalizability of TFBS informativeness as determined in the PDX samples. FIG. 13B shows cfDNA TSS profile shape similarity calculated for PDX samples (top row) and patient samples with tissue RNAseq data (bottom row) and for all sites (left column) or only sites with Mann-Whitney U test p value<0.01 (i.e., highly informative sites, right column), with linear regression fit shown as black line, Pearson's r and p value. Selection of informative sites in PDX samples improved the correlation between profile shape similarity metric and gene expression in patient samples, supporting the generalizability of TSS informativeness as determined in the PDX samples.

FIG. 14 depicts hierarchical clustering of cfDNA nucleosome profile shape similarity values at an inclusive set of TSSs. Hierarchical clustering of cfDNA nucleosome profile Euclidean distance ratios at all TSSs which are highly informative and associated with genes that are correlated to SCLC TF expression in external data (n=106) in neuroendocrine PDX samples (n=20). First column: Dendrograms and heatmap of clustered TSS Euclidean distance ratios. Second column: Pearson correlations between transcript level of gene of interest (y axis) and key SCLC TFs (x axis) in independent SCLC cell line RNAseq data. Bottom row: Tissue transcript levels for key TFs in PDX samples. Unsupervised clustering of selected highly informative TSSs groups samples by tissue TF expression and groups of genes by correlation with key SCLC TFs.

FIG. 15A to 15F depict phenotype prediction model characteristics and results in PDX and patient samples. FIG. 15A is a table of phenotype prediction model (SCLC vs NSCLC or TF activity) characteristics and prediction accuracy metrics. For SCLC vs NSCLC prediction models, SCLC samples were considered “positive” and NSCLC samples “negative.” FIG. 15B shows cfDNA and tumor characteristics for SCLC patient samples without matched tissue (n=46). Samples were grouped into columns by PDX/patient and SCLC or not SCLC. Top row: Dendrogram and heatmap of hierarchically clustered cfDNA nucleosome profile TF scores predicted by TF score estimators. Second row: Ground truth/predicted REST activity shown for neuroendocrine samples. Third row: Ground truth/predicted SCLC subtype shown for neuroendocrine samples. Fourth row: Ground truth/predicted histology from histology classifier (FIG. 4B) is shown for reference. FIG. 15C depicts distribution kernel density estimates of predicted TF scores in patient samples without matched tissue by histology (n=9 NSCLC and 46 SCLC in each comparison), with two sided Mann-Whitney U test p value. FIG. 15D depicts a number of SCLC patient samples without matched tissue (n=46) predicted positive or negative for a given TF based on thresholding of TF scores, with percent shown overlaid/adjacent. FIG. 15E shows the NEUROD1 TF score estimate versus ASCL1 TF score estimate for patient samples with matched tissue (left panel) and patient samples without matched tissue (right panel), colored by tissue subtype and size scaled by ichorCNA-estimated tumor fraction.

FIG. 15F shows the relative cfDNA TF score (ASCL1/ASCL1+NEUROD1) in SCLC cfDNA samples by sample source (PDX or patient) and tissue SCLC subtype. Dotted lines are shown at ratios of 0.25 and 0.75. Most patient samples exhibited relatively extreme (ratio<0.25 or >0.75) relative TF scores.

FIG. 16 is a listing of genes targeted by the methods disclosed herein.

FIG. 17 is a sample of the gene expression for the PDX and patient samples showing the type of cancer as determined by histology and expression data for ASCL1, ATOH1, REUROD1, POU2F3, and REST.

FIG. 18 is a table showing the number of features as a total number of 15 bp genomic bins that were independently considered in the predictive model.

### DETAILED DESCRIPTION

Small cell lung cancer (SCLC) is an aggressive neuroendocrine neoplasm with poor clinical outcomes and a paucity of therapeutic advances. While initially sensitive to platinum/etoposide chemotherapy, emergence of chemotherapy resistant disease is rapid. SCLC is typically diagnosed using fine needle aspiration of mediastinal lymph nodes leading to inadequate clinical material to link biological features of SCLC to treatment responses. Methods to non-invasively profile SCLC tumors are greatly needed to advance patient care. As above, SCLC exhibits heterogeneity in driver gene mutations and distinct molecular subtypes characterized by activation of transcription factors (TFs) such as ASCL1, NEUROD1, POU2F3, and REST. Regulation of other key genes, including the MYC family, the NOTCH pathway, and cell surface markers such as DLL3 and SEZ6 that are targets of antibody-drug conjugates or BiTEs make up additional phenotypes that could inform clinical decisions in SCLC. Finally, trans-differentiation of non-small cell lung cancer (NSCLC) into SCLC can occur as a mechanism of resistance to targeted therapies and remains a challenge to detect clinically, requiring improved methods to monitor such transformations and to further discern SCLC subtype following transformation.

SCLC does not have “driver mutations” like NSCLC. A driver mutation is typically viewed to mean a mutation that is oncogenic, and targetable. NSCLC is known to have driver mutations making the treatments for NSCLC very different from treatments for SCLC.

SCLCs are known to be driven by differences at the epigenetic level, meaning that rather than the DNA sequence of the genes being changed, other factors influence which genes are expressed and which are not. Among these other factors are transcription factors which help to control gene expression. Four subtypes of SCLC are currently recognized. SCLC-A (wherein the dominant transcription factor is ASCL1), SCLC-N (wherein the dominant transcription factor is NEUROD1), SCLC-P (wherein the dominant transcription factor is POU2F3), and SCLC-I (wherein no particular transcription factor is dominant; however, the tumor exhibits a number of inflammatory features including, for example, immune checkpoints, T cells, and other immune cells). As such, the various subtypes respond to some of the same, but in many cases different treatments. For example, SCLC-A is typically is treated with BCL2 inhibitors, DLL3 targeted drugs, HDAC inhibitors, LSD1 inhibitors, Cisplatin and PARP inhibitors, and/or labetuzumab govitecan; SCLC-N is typically treated with therapies targeting MYC, IMPDH inhibitors and/or somatostatin analogues; SCLC-P is typically treated with therapies targeting MYC, PARP inhibitors with anti-metabolites containing nucleoside analogues and anti-folates, IGF1R inhibitors, MICA inhibitors, and/or mSWI/SNF ATPase degraders; and SCLC-I is typically treated with therapies targeting MYC, IMPDH inhibitors, BTK inhibitors, MICA inhibitors, immune checkpoint inhibitors, and/or HDAC inhibitors.

Recognizing the need to phenotype SCLC, the present disclosure provides a targeted capture cell-free DNA (cfDNA) sequencing assay, SCLCpheno-seq, that profiles genomic and transcriptional features in plasma samples. The assay analyzes circulating tumor DNA (ctDNA) fragment patterns to infer nucleosome profiles at key transcription start sites (TSSs) for thousands of genes. SCLCpheno-seq also profiles lung cancer for exonic mutations in cancer driver genes, allowing for “all-in-one” tumor phenotyping from a biological sample, such as blood. The example assays provided herein show that the application of SCLCpheno-seq to plasma cfDNA from SCLC and NSCLC transformation in xenograft models and clinical patient samples faithfully capture TF and gene activity for SCLC transcriptional subtyping and histological classification. The results of the assays can be used by a skilled practitioner to modify or adjust patient treatment depending on the outcome to obtain better treatment results for a patient.

In the present disclosure, cfDNA nucleosome profiling has been further adapted for non-invasive TF activity and gene expression inference in SCLC. Previous nucleosome profiling studies have either used whole genome sequencing (WGS) at varying depths or have used targeted sequencing at a small number of sites of interest (35). A novel approach has been developed and described herein which comprises targeted sequencing of thousands of genomic loci in cfDNA to identify nucleosome profiling signals. This method, which is termed SCLCpheno-seq, facilitates detailed investigation of nucleosome profile patterns at individual genomic sites. Furthermore, because SCLCpheno-seq analyzes native cfDNA, it is modular and can be seamlessly integrated with conventional targeted sequencing for mutation detection. First, targeted cfDNA nucleosome profiling was validated to reliably detect signals of TF activity and gene expression. Then nucleosome profiling signals at individual genomic sites were examined to generate key insights regarding site-to-site heterogeneity. Finally, high performance of targeted nucleosome profiling was demonstrated for two key applications: (1) discrimination of tumor histology (NSCLC vs SCLC), which could be used to monitor for histologic type switching during targeted therapy of NSCLC, a well-described resistance mechanism, and (2), inference of TF activity to predict tumor SCLC subtype. Overall, SCLCpheno-seq is a practical and accurate method for non-invasive prediction of key tumor features in lung cancer patients with the potential to substantially accelerate the arrival of improvements in patient care.

The development of the disclosed cfDNA assay addresses major deficiencies in SCLC precision medicine, including applications in monitoring and predicting therapeutic outcomes: (i) discrimination of tumor histology (NSCLC vs SCLC) to monitor emergent resistant phenotypes in “real-time”, which can be used to monitor for histologic transformation during targeted therapy of NSCLC and allocate the appropriate therapy to improve treatment outcomes; (ii) classification of SCLC molecular subtypes when tissue biopsies are not routinely available. The disclosed assay and methods examine the phenotypes with the most clinical impactful. The ability to accurately determine genomic and transcriptional subtypes from cfDNA will be critical for biomarker discovery in correlative/translational studies for clinical trials and the development of future companion diagnostics for new SCLC targeted therapies. cfDNA nucleosome profiling is a practical and accurate method for non-invasive prediction of key tumor features in lung cancer patients.

The nucleosome profiling method used herein is generally disclosed in WO 2022/217096 (incorporated by reference herein in its entirety). The method (Griffin) comprises the following steps:
receiving, by a computing system, sequence read data, wherein the sequence read data includes a plurality of fragment reads, wherein each fragment read has a fragment length and a GC content indicating a percentage of bases in the fragment read that are G or C;
determining, by the computing system, GC bias values for each fragment read based on the fragment length and the GC content of the fragment read;
generating, by the computing system, a genomic coverage distribution that is adjusted for GC bias using the sequence read data and the GC bias values; and
predicting, by the computing system, the cell type based on the genomic coverage distribution.

The method proceeds with determining the genomic regions of interest and filtering to identify cell-type-informative sites. Any suitable technique for determining and filtering cell-type-informative sites can be used, and different techniques will likely be used for different types of cancer, different molecular subtypes of a cancer type, different tissues, different cell types, and different types of assays. Next, a GC frequency matrix is determined for combinations of fragment lengths and GC content. For certain sequencing technologies, fragments having certain amounts of G and C bases (“GC content”) will be overrepresented in the sequence read data. This bias is not constant, as fragments of different sizes will have different GC biases. Because sequence read data from cell-free DNA fragments typically includes short fragments of many different lengths, establishing a GC frequency matrix that specifies expected proportions of GC content for various different fragment lengths allows sequence read data to be properly corrected for the GC bias, and for meaningful signals to be obtained from sequence read data that would otherwise be too noisy. One will recognize that the actions described may be performed on reference genome data before obtaining a sample or sequence data to be analyzed.

Next, the sequence read data is received. In some embodiments, the sequence read data represents sequence reads generated for a sample obtained from a subject. In some embodiments, the sequence read data may be obtained from an archive or other previously obtained sample. The GC frequency matrix is used to determine GC bias values for the sequence read data. Any suitable technique may be used here. The GC bias values are used to generate a genomic coverage distribution of the sequence read data for the cell-type-informative sites. Again, any suitable technique may be used. Next, features are extracted from the genomic coverage distribution. Any features suitable for use with a classifier model may be extracted and may depend on the type of classifier model used, the assay that generated the sequence reads, and/or the cell type (e.g., type of cancer, cancer subtypes, tissue, or cell type) to be detected. Mean coverage may be extracted by determining the mean coverage in a window around an informative site. The window around the informative site for determining mean coverage may be any suitable size, including but not limited to a range from 1800-2200 bp (from +/−900 bp to +/−1100 bp). One non-limiting example of a suitable size for the window for determining mean coverage is 2000 bp (+/−1000 bp).

Central coverage may be extracted by determining the mean coverage in a smaller window around the informative site. The window around the informative site for determining central coverage may be any suitable size, including but not limited to a range from 40-80 bp (from +/−20 bp to +/−40 bp). One non-limiting example of a suitable size for the window for determining mean coverage is 60 bp (+/−30 bp).

Amplitude may be extracted by trimming the genomic coverage distribution to an area that includes a given number of peaks (such as an area of +/−960 bp that contains 10 peaks), performing a fast Fourier transform, and taking the magnitude of a frequency based on the given number of peaks (e.g., the 10th frequency for the area that contains 10 peaks). The features are provided as input to a classifier model to predict the tumor subtype. Any suitable classifier model may be used. Once the cancer subtype is predicted by the classifier model, the method then terminates. Naturally, in some embodiments, further action may be taken once the cancer subtype is determined, including but not limited to an appropriate cancer diagnosis, identifying cancer subtype change or switch, recommending a new course of treatment, altering an existing course of treatment, or any other appropriate action.

In some embodiments, the method is performed a plurality of times. Accordingly, the method can be a method of monitoring for the presence and/or identity of cancer in the patient. The cancer cell(s) detected in the patient at each performance of the method can be further characterized. For example, the cell(s) can be monitored over time using this method to determine a cancer subtype or phenotype of the detected cancer cell(s) based on the prediction provided by the computing system. In some embodiments, the method further comprises detecting a change in phenotype of the detected cancer cell(s) over time. For example, as described in more detail below SCLC can progress from one subtype to another during the course of disease or NSCLC and transdifferentiate into SCLC. In certain embodiments, cancer cells can evolve and essentially switch between characterized subtypes. These changes can be associated with changes in malignancy and/or responsivity to various treatments, all of which can be detected given the demonstrated sensitivity of the Griffin workflow.

Monitoring and documenting such changes over time can inform a requirement for modification of therapy to optimize the outcome. As a non-limiting example, non-small cell lung cancer (NSCLC) can be monitored for trans-differentiation to small cell lung cancer (SCLC). Alternatively, SCLC subtypes can be monitored for trans-differentiation to distinct subtypes. In some embodiments, the method can be performed starting before or during the course of treatment for cancer. Accordingly, the cancer can be monitored for responsivity to the treatment, or for changes in phenotype during the course of treatment. These characteristics can inform any appropriate adjustments to the treatment regimen. In some embodiments, the method comprises implementing a treatment or treatment change based on the monitored status of the cancer cells as determined by the method. In another aspect, the disclosure provides a method of determining a cancer subtype of a target cancer cell from a sample comprising cell-free DNA derived from the target cancer cell. The method comprises:
obtaining sequence read data generated from the sample comprising cell-free DNA;
performing the computer-implemented method described in more detail above (and which is incorporated into this aspect in all of its embodiments); and
determining the cell type of the target cancer cell based on the predicted cancer 25 subtype provided by the computing system.

The sample can be a biological sample from the patient, e.g., a patient with cancer or suspected to have cancer. Exemplary biological samples are described in more detail below. In some embodiments, the method comprises obtaining the biological sample from the subject and/or generating the sequence read data from the sample, according to standard techniques appropriate for the desired sequencing platform and/or targeted capture technology.

In another embodiment, the cancer is characterized as metastatic lung cancer. In a further embodiment, determining the subtype of the lung cancer comprises determining whether the cancer is small cell lung cancer (SCLC) or non-small cell lung cancer (NSCLC). As indicated above, the input sequence read data can be generated from a variety of platforms and with a variety of techniques, including whole genome analysis. In the example presented herein, it has been established that whole genome analysis, however, is not required. Instead, a panel of genomic targets deemed relevant to distinguishing NSCLC from SCLC and subtyping SCLC was designed.

Accordingly, in some embodiments, the lung cancer is further subtypes using sequence read data generated from a panel of genomic targets. In some embodiments, the panel of genomic targets comprises transcription factor binding sites (TFBSs) of one or more transcription factors associated with a designated subtype that is the subject of analysis, e.g., SCLC. For example, for subtyping SCLC, the one or more associated transcription factors comprise one or more of ASLC, ATOH1, NEUROD1, POU2F3, REST, and the like. In such embodiments, the method comprises determining the nucleosome occupancy of the TFBSs using any appropriate technique (e.g., CUT & RUN, and the like). The TFBSs can be identified by ChIP-seq data, or similar techniques known in the art. Candidate TFBSs can be retained in the panel if they are proximal to a transcription start site (TSS) of a gene associated with lung cancer, or the subtype of lung cancer that is of interest in the subtyping. In this regard, the term proximal can mean within a proximity that the TFBSs is functionally influential on the start of transcription at the TSS. In some instances, the functional influence or relationship can be established if the TSS is the closest TSS to the TFBS. In other embodiments, the panel of genomic targets comprise transcription start sites (TSSs) for one or more markers associated with lung cancer (or the specific subtype of lung cancer that is of interest). In such embodiments, the method comprises determining the nucleosome occupancy of the TSSs through known techniques.

The biological sample described herein can be any sample obtained from a subject that is likely to have cell free DNA. Illustrative, non-limiting examples encompassed by the disclosure include the sample is blood, plasma, or serum, which are particularly useful to assess cfDNA and ctDNA from a subject. In any embodiment of the foregoing aspects relating to detection or assessment of cancers in a subject, the methods can further comprise obtaining the biological sample from the subject. Additionally, for a subject that is determined to have cancer or a cancer subtype at any time, the method can further comprise prescribing appropriate treatment or actively treating the subject appropriately based on the determination of the cancer type or subtype according to accepted practice in the medical field for the determined cancer.

In any aspect described herein, the described method can be performed multiple times to provide multiple assessments. This can be useful to provide methods for monitoring the presence or evolution of cell types or subtypes from a source. For example, the methods can be performed from sequence read data obtained from biological samples obtained from a subject before and/or for time points at or after initial diagnosis of cancer.

In the present application, a custom target sequencing panel was designed to simultaneously identify somatic mutations and infer transcription factor activity and gene expression from plasma cfDNA. In a particular embodiment 842 genes were assembled from various sources, including, for example, published cancer gene panels, genes known to be frequently mutated in SCLC, other sources including, those known from functional studies can also be used.

To infer the activity of transcription factors (TFs) key in SCLC, windows of 1 kb around Transcription Factor binding sites (TFBS) in ASCL1 (590), NEUROD1 (414), POU2F3 (640), and REST (781) that were identified using available sequencing and gene expression data. Regarding putative binding sites for ASCL1 and NEUROD1, only sites of at least 20 base pairs (bp) in length after the intersection of lists of peaks across examined cells lines were retained. Putative binding sites were defined as associated with a differentially expressed gene if they were either within 10 kb of a differentially expressed gene TSS, or if the closest gene TSS on the same chromosome was differentially expressed (regardless of distance), and only differentially expressed gene-associated putative binding sites were retained. For ROU2F3, putative bindings sites were annotated as being associated with a differentially expressed gene and filtered as described above for ASCL1 and NEUROD1. For REST, putative binding sites were defined as peaks observed in a least five experiments in a sequence data base, for example, the GTRD human ChIP-seq data base. SCLC cell line gene expression data was mined to create a coarse list of REST-associated differentially expressed genes. REST-associated differentially expressed genes were defined as the 250 genes that were most significantly more highly expressed in REST-high cell lines. Putative REST binding sites were annotated as being associated with a differentially expressed gene and filtered as described above.

Transcription start sites were selected from various lists well known in the art, such as, the human genome version of GRCh38.p12). TSSs residing on alternative contigs or chromosome Y were excluded. TSSs were required to have at least one of the following properties: i) transcript support level equal to 1 according the Gencode v31; ii) a single exon gene; iii) association with the same genes that were defined as SCLC-TF associated when designing the TFBS component of the panel, or iv) presence in the MSigDB v7.0 Hallmark pathways list. In the specific embodiments disclosed herein a list of 36,379 TSSs corresponding to 18,030 unique genes were obtained. Sites were padded by 100 bp upstream and 260 bp downstream and these intervals were used to obtain a probe set by known methods that adequately targeted (defined as continuous probe coverage from 90 bp upstream to 245 bp downstream of the TSS) for a total of 35,917 TSSs corresponding to 17,921 genes. Desired genes and genomic regions surrounding TFBSs and TSSs of interest, as defined above, were used for custom targeted panel design and synthesis by known methods. In a specific embodiment disclosed herein coding sequence and TFBS regions were targeted together in one 4.5 Mb panel and TSS regions were targeted in a second 9.2 Mb panel.

Genomic DNA can be extracted from a biological sample by known methods. A biological sample can be a blood or plasma sample. In a specific embodiment disclosed herein genomic DNA was extracted from buffy coat and cell-free DNA was extracted by known methods. After isolation, DNA concentration was measured, and size distribution was assessed.

Gene sequencing data can be obtained using methods well known in the art, including, for example, ChIP-seq. Plasma cfDNA sequencing libraries were constructed. Buffy coat genomic DNA was fragmented mechanically to a goal modal fragment size of approximately 250 nucleotides.

To infer the expression of single genes, windows of around transcription start sites (TSS) were examined. In a specific embodiment 360 bp windows around 35,917 TSSs corresponding to 17,921 genes were targeted. In total, the panel targeted 13.7 Mb with 120 bp probes. The targeted cfDNA sequencing approach was applied to plasma from mice harboring SCLC or NSCLC PDX models. In addition, the targeted panel was applied to plasma samples from patients with SCLC, patients with NCLC, and to individuals without cancer.

In certain embodiments, to facilitate interpretation of cell-free DNA nucleosome profiling, a stepwise labeling scheme is developed for transcription factor activity for small cell lung cancer and large cell neuroendocrine carcinoma patient-derived xenograft models and small cell lung cancer patient samples. In certain specific embodiments, activity of key small cell lung cancer transcription factors, including but not limited to: POU2F3, ASCL1, NEUROD1, ATOH1, and REST, in samples with available cell-free DNA data is assigned as follows: (1) samples with all transcription factor transcripts<5 are labeled “pan-low”; (2) samples with POU2F3 transcript>5 are labeled POU2F3-active; (3) the remaining samples are examined for ASCL1, NEUROD1, and ATOH1 activity; (4) samples with ATOH1 transcript>5 are labeled ATOH1-active; (5) because ASCL1 and NEUROD1 can be co-expressed, samples with ASCL1, NEUROD1, or ASCL1 and NEUROD1 transcript>5 are subjected to unsupervised hierarchical clustering of the transcript levels of a group of 526 ASCL1 and NEUROD1-associated genes, to produce two main clusters with divergent ASCL1 versus NEUROD1 expression, in which samples are labeled as ASCL1-active or NEUROD1-active based on relative expression of ASCL1 and NEUROD1 in the resulting clusters; and (6) REST activity is labeled based on sample grouping after unsupervised hierarchical clustering of samples by REST target gene transcript levels. In some embodiments, clustering is performed with the linkage method, for example, the scipy software package (v1.7.1) with method=“ward” and metric=“Euclidean” can be used. In some embodiments, transcript levels are considered in units of log₂(TPM+1). In certain specific embodiments, transcription factor activity for ASCL1, NEUROD1, and POU2F3 in samples with immunohistochemical data is classified as positive, focal, or negative. In certain embodiments, subtype labels are generated as the union of all transcription factors that are considered active, including positive or focal for patient samples with immunohistochemical data only.

In a specific embodiment, analysis of targeted cell-free DNA nucleosome profiles at transcription start sites for evidence of tumor cell gene expression includes: (1) focusing on a set of 13,240 transcription start sites selected for transcript abundance, available gene expression data, and adequate coverage in cell-free DNA libraries; (2) examining the coverage at individual transcription start sites in the data for single gene expression inference by calculating the correlation between the transcription start site “amplitude,” which is the magnitude of the coverage difference between the +120 and −45 positions at an individual transcription start site, and gene transcript level, and by calculating the fragment length entropy.

In certain embodiments, analysis of targeted cell-free DNA nucleosome profiles includes analysis of nucleosome occupancy. In one embodiment, analysis of nucleosome occupancy includes: (1) assigning one transcription start site to each gene with available expression data; and (2) determining the distance from each transcription start site to the nearest CpG island. In a specific embodiment, assigning transcription start sites to genes uses, for example, CCLE transcript-specific gene expression data from the 2021Q1 CCLE data repository to select, for each gene, a single transcription start site associated with the most highly expressed transcript across small cell lung cancer cell lines. The following is retained in the result: (a) a transcription start site for genes in which the transcription start site associated with the most highly expressed transcript is not covered by the targeted panel but a transcription start site corresponding to a similarly expressed transcript is; and (b) genes with only one transcription start site that is covered by the targeted panel, even when that site does not correspond to the most highly expressed transcript in the CCLE data. In one embodiment, the resulting list of one-to-one transcript start site-to-gene mappings is intersected with (a) transcript start sites that are successfully analyzed by the Griffin nucleosome occupancy profiling, (b) genes for which transcript levels are estimated in all the patient-derived xenograft and patient samples in which RNA sequencing has been performed, and (c) sites at which the entropy feature could be calculated, to generate a curated list of transcription start site-to-gene pairings for subsequent analyses. In one embodiment, determining the distance from each transcription start site to the nearest CpG island includes (1) downloading known CpG island locations from a data base, for example, the UCSC table browser (table cpgIslandExt for UCSC genome version hg38, most recently updated on 3/13/2020); (2) calculating the distance from each transcription start site to the nearest CpG island using the “closest” function in, for example, the Bedtools package (v2.30.0), and (3) labeling transcription start sites CpG proximal if the nearest CpG island is <1 kb away.

In certain embodiments, analysis of targeted cell-free DNA nucleosome profiles includes calculating descriptive features from the nucleosome profiles. In one embodiment, descriptive features from the nucleosome profiles include: (1) the transcription factor binding site central amplitude, (2) the transcription start site amplitude, (3) the transcription start site fragment length entropy, (4) the transcription factor binding site k-means clustering and profile shape similarity metric, and (5) the transcription start site k-means clustering and profile shape similarity metric.

In a specific embodiment, the transcription factor binding site central amplitude is calculated as the arithmetic difference between the mean coverage across two windows flanking the transcription factor binding site (−195 to −120 bp and 120 to 195 bp) and the mean coverage at the transcription factor binding site (−30 to 30 bp).

In a specific embodiment, the transcription start site amplitude is defined as the arithmetic difference between the coverage at the +120 position and the −45 position relative to the transcription start site. Calculating the transcription start site fragment length entropy at individual transcription start sites includes: (a) tabulating the distribution of aligned fragment lengths in 5 bp bins; (b) defining aligned fragments as fragments with mapping quality of at least 20 in both reads ranging in length from 15 to 500 bp that overlap any of the region 90 bp upstream to 240 bp downstream of each transcription start site; (c) calculating the entropy of these binned length distributions using, for example, the scipy.stats.entropy function; and (d) Z score-normalizing the entropy scores within each sample.

In a specific embodiment, calculating the transcription factor binding site k-means clustering and profile shape similarity metric includes: (a) performing, at each TFBS, k-means clustering (k=2) of the nucleosome occupancy profiles of the patient-derived xenograft samples; (b) trimming the nucleosome occupancy profiles to the central 720 bp to minimize the contribution of noisier signal at the edges of captured windows, and performing clustering with, for example, the sklearn.cluster.KMeans module of the scikit-learn package with random_state=0; (c) calculating the adjusted Rand index at each transcription factor binding site as an indicator of the extent to which variation in nucleosome occupancy profile at that site is related to tissue expression of the corresponding transcription factor; (d) classifying patient-derived xenograft models as positive or negative for a given transcription factor as described in the above embodiment; (e) calculating the adjusted Rand index of the clustering assignment using, for example, the sklearn.metrics.adjusted_rand_score module of the scikit-learn package, with sites that have an adjusted Rand index of at least 0.5 to be considered as informative; (f) using the results of the clustering at each site to calculate average nucleosome profiles within each of the two clusters, and labeling average profiles as “high” or “low” expressing of the transcription factor of interest based on the mean relative expression of the transcription factor in samples in each cluster; and (g) calculating, for each sample at each site, the Euclidean distance between the occupancy profile observed and each of the two average profiles, and also calculating the log₂ transformed ratio of the two Euclidean distance metrics, where the distance to the “low” expressing cluster is in the numerator and the distance to the “high” expressing cluster is in the denominator.

In one embodiment, the k-means clustering and profile shape similarity metric for transcription start site is calculated as for individual transcription factor binding sites as described in the preceding embodiment, with the following modifications: (a) trimming nucleosome occupancy profiles to a −60 to +165 bp window relative to the transcription start site to minimize the contribution of noisier signal at the edges of captured windows; (b) performing, for each transcription start site of interest, k-means clustering with k=2 of the nucleosome occupancy profiles in the PDX samples; (c) testing, for each site, whether the clustering yields two distinct expression states by performing a Mann-Whitney U test of the log₂(TPM+1) expression values for the gene of interest in the samples in each of the two clusters; (d) filtering sites based on the Mann Whitney U test p values to identify sites where the coverage profiles significantly discriminated tissue gene expression in the patient-derived xenograft models; (e) to minimize the inclusion of spuriously informative sites, requiring the difference in mean expression between the two groups of samples generated by clustering to be at least four-fold; and (f) calculating, for each sample, the Euclidean distance of the coverage profile at that transcription start site to the mean coverage profile of each of the two clusters, and also calculating a single metric of relative distance as the log₂ of the ratio of the distance of the coverage profile to the low expression cluster mean over the distance of the coverage profile to the high expression cluster mean.

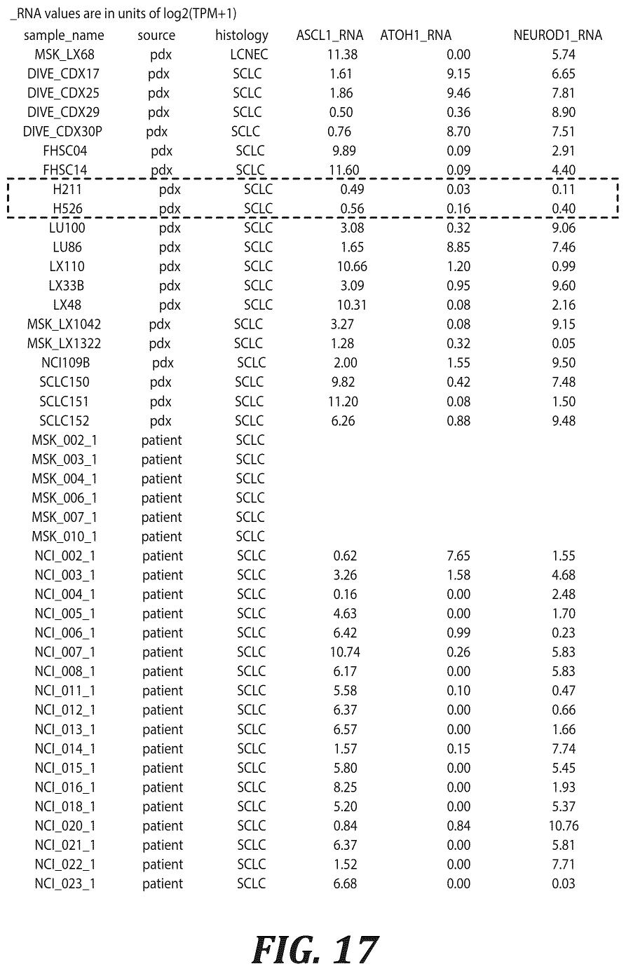

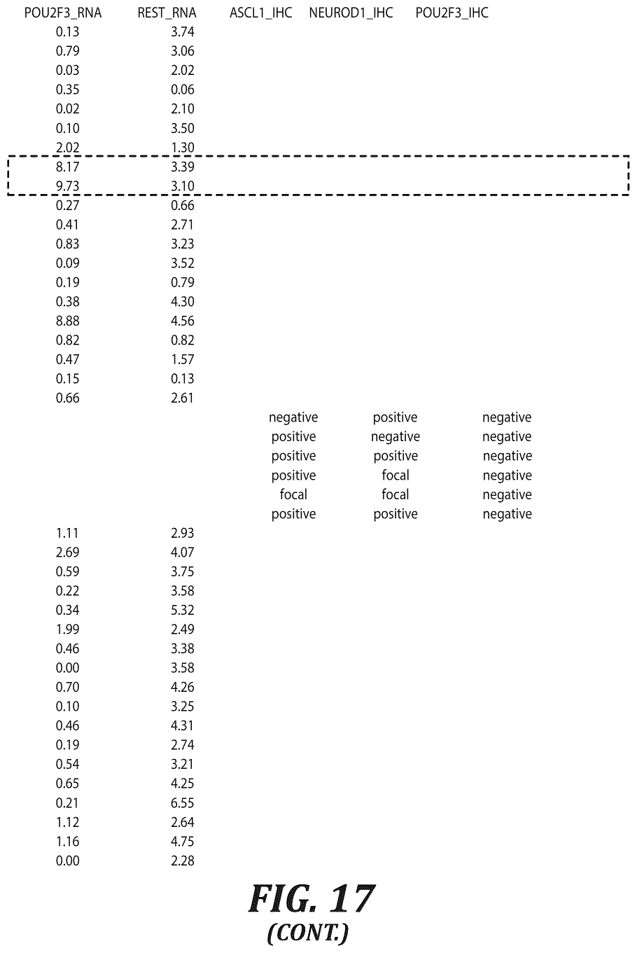

In a specific embodiment, analyzing the targeted nucleosome profiles to predict key tumor features in lung cancer patients includes identifying genes that are correlated with key small cell lung cancer transcription factors in the nucleosome profiles. In one embodiment, identifying genes that are correlated with key small cell lung cancer transcription factors includes: (1) using the small cell lung cancer cell line gene expression data from for example, CCLE (2021 Q2 dataset), correlating individual gene expression values with the expression of the key small cell lung cancer transcription factors, including but not limited to: ASCL1, ATOH1, NEUROD1, and POU2F3 (Sample gene expression information is provided in FIG. 17); and (2) defining transcription factor-associated genes as genes with a Pearson correlation coefficient greater than 0.6 with a given transcription factor.

In one embodiment, analyzing the targeted nucleosome profiles to predict key tumor features in lung cancer patients includes identifying genes that are differentially expressed between non-small cell lung cancer and small cell lung cancer cell lines. In one embodiment, identifying genes that are differentially expressed between non-small cell lung cancer and small cell lung cancer cell lines includes categorically labeling genes as expressed or not expressed based on a log₂(TPM+1) cutoff of 1, and testing differential expression status using Chi-square, where genes with differential expression false discovery rate<0.1 (Benjamini-Hochberg) are considered histology-specific.

In another aspect, the disclosure provides a method of predicting lung cancer histologic type, such as distinguishing small cell lung cancer from non-small cell lung cancer using targeted cell-free DNA nucleosome profiling. The method comprises:
receiving, by a computing system, cell-free DNA sequencing data from small cell lung cancer and non-small cell lung cancer patient-derived xenograft models or patients;
formulating, by a computing system, a probabilistic framework that analyzes cell-free DNA nucleosome profiles that simultaneously estimates the tumor fraction and histologic composition of a patient plasma sample using highly informative transcription factor binding sites;
applying, by the computing system, the probabilistic framework to the received cell-free DNA sequencing data, and
   estimating, by the computing system, a “histology score,” representing the histologic composition as a continuous value from 0 (non-small cell lung cancer-like) to 1 (small cell lung cancer-liked).

In another aspect, the disclosure provides a method of inferencing small cell lung cancer subtype in patient samples using transcription factor activity prediction. The method comprises:
receiving, by a computing system, cell-free DNA sequencing data from small cell lung cancer and non-small cell lung cancer patient-derived xenograft models or patients;
formulating, by a computing system, a probabilistic framework that analyzes cell-free DNA nucleosome profiles to predict the activity of the small cell lung cancer transcription factors;
formulating, by the computing system, a prediction model that jointly estimates cell-free DNA tumor fraction and a “transcription factor score,” ranging from 0 (transcription factor-negative) to 1 (transcription factor-positive), for each transcription factor; and
applying, by the computing system, the probabilistic framework to the received cell-free DNA sequencing data to predict transcription factor activity in the patient samples.

In some embodiments, formulating a probabilistic framework for analyzing targeted nucleosome profiling includes: (1) designing a probabilistic model to estimate the composition of patient samples with respect to lung cancer histology and small cell lung cancer transcription factor activity; and (2) training each probabilistic model with samples that represent the phenotypic states of interest. In one embodiment, designing the probabilistic model includes: (a) modeling the cfDNA nucleosome relative coverage profiles observed in patients as a linear combination of the coverage profiles in cell-free DNA derived from “healthy” (non-malignant) cells only and from tumor cells only; (b) modeling the tumor cells only component as a linear combination of cell-free DNA coverage profiles derived from one of two phenotypic states of interest, such as NSCLC or SCLC, or TF-positive or TF-negative; (c) defining, for each genomic position “i” within the coverage profile, the expected coverage (μᵢ) in a patient samples as a function of “a,” which is the fraction of tumor cell-free DNA that is derived from the phenotypic state, modeled by the following equation:

$${μ}_{i} ( α , s ) = ( 1 - α ) {μ}_{i , H} + ( α \times s ) {μ}_{i , S} + ( α \times ( 1 - s ) ) {μ}_{i , N}$$

(d) determining the expected coverage values for each component (μ_{i,H}, μ_{i,S} and μ_{i,N}) by the mean coverage values observed in data in the non-malignant patients or in the patient-derived xenograft samples, i.e., calculating the “healthy” coverage mean (μ_{i,H}) using the non-malignant patient samples, calculating μ_{i,S} using the phenotype-positive patient-derived xenograft samples, and calculating μ_{i,N} using the phenotype-negative samples; (e) calculating the standard deviation for each feature (σi) as the mean of the standard deviations from each of the three states as follows:

$${σ}_{i} = \frac{{σ}_{i , H} + {σ}_{i , S} + {σ}_{i , N}}{3}$$

(f) using the above relationships, modeling the likelihood of a given tumor fraction (α) and subtype fraction (s) given the vector of features (coverage at each position, F_{0:T}) observed in cell-free DNA from a particular sample, as follows:

$$ℒ ( α , s \vert {F}_{0 : T} ) ∝ {\prod }_{i = 0}^{T} 𝒩 ( {F}_{i} \vert {μ}_{i} ( α , s ) , {σ}_{i} )$$

(g) calculating the probability of each feature Fi being derived from a normal distribution with mean μᵢ(α, s) and standard deviation σi, and then taking the product of those probabilities as an estimate of the relative likelihood of a given a and s giving rise to the observed signal; (h) finding the a value and s value that have the highest likelihood of creating the observed data taking the −log₂ of the likelihood value and using a minimize function, for example, the minimize function from scipy.optimize (version 1.7.1) to estimate a and s simultaneously. In one embodiment, this framework as described is used to generate six models: the first model predicts the histology (small cell lung cancer versus non-small cell lung cancer), in which case s is the “histology score;” the other five models predict transcription factor activity in small cell lung cancer samples, including but not limited to transcription factors ASCL1, NEUROD1, ATOH1, POU2F3, and REST, in which s is the “transcription factor score.”

In certain embodiments, each probabilistic model was trained with samples that represent the phenotypic states of interest includes: (a) for all probabilistic models, using non-malignant patient samples to estimate the mean feature values in the “healthy” component of cell-free DNA (μ_{i,H}); (b) using the patient-derived xenograft models to estimate the mean feature values for a tumor with (μ_{i,S}) or without (μ_{i,N}) the phenotype of interest, with the specific patient-derived xenograft models used depending on the phenotype being predicted; (c) for the small cell lung cancer versus non-small cell lung cancer probabilistic model, using the small cell lung cancer patient-derived xenograft samples to estimate the mean for the phenotype of interest (μ_{i,S}), and both lung adenocarcinoma and squamous carcinoma patient-derived xenograft samples are used to estimate the mean for the non-phenotype of interest (μ_{i,N}); (d) excluding large cell neuroendocrine carcinoma patient-derived xenograft sample from the training set due to shared features with both small cell lung cancer and non-small cell lung cancer; (e) for the transcription factor activity predictors, using only the small cell lung cancer patient-derived xenograft samples in the training set; (f) using the transcription factor-positive models to calculate (μ_{i,S}), and using the transcription factor-negative models to calculate (μ_{i,N}); (g) for preliminary testing, training and testing models on patient-derived xenograft and non-malignant patient samples using leave-one-out cross-validation; and (h) for testing on small cell lung cancer and non-small cell lung cancer patient samples, training models on the entire set of patient-derived xenograft and non-malignant patient samples.

In some specific embodiments, to train each probabilistic model, a combination of transcription factor binding sites and transcription start sites that are informative and relevant to the phenotypes of interest is used for each probabilistic model, such as: (a) using the Griffin relative coverage within each transcription factor binding site in 15 bp bins from −360 bp to 360 bp around the transcription factor binding site; and (b) using the Griffin relative coverage within each transcription start site in 15 bp bins from −60 bp to 165 bp around the transcription start site.

In one embodiment, training the small cell lung cancer versus non-small cell lung cancer probabilistic model uses all transcription factor binding sites that are determined to be informative, and all transcription start sites that are determined to be informative and coarsely differentially expressed between small cell lung cancer and non-small cell lung cancer cell lines. In some embodiments, the determination of whether transcription factor binding sites are information is based on their k-means clustering, as described in the above embodiment. In some embodiments, the determination of whether transcription start sites are information and coarsely differentially expressed between small cell lung cancer and non-small cell lung cancer cell lines is based on their k-means clustering and profile shape similarity metric and also the identification of genes that are differentially expressed between small cell lung cancer and non-small cell lung cancer cell lines, as described in the above embodiments.

In some embodiments, in training the probabilistic models to predict transcription factor activity in small cell lung cancer samples, the transcription factor activity prediction is restricted to informative transcription factor binding sites associated with a given transcription factor and informative transcription start sites associated with genes with expression correlated to small cell lung cancer transcription factor expression in small cell lung cancer cell line RNA sequence data, as described in the above embodiments on k-means clustering and profile shape similarity metric calculation for transcription factor binding sites and transcription start sites and on identifying genes correlated with key small cell lung cancer transcription factors.

In some embodiments, training the probabilistic models includes selecting a threshold for the models. In some embodiments, the optimal thresholds for all models are selected by calculating the F1 Score in the patient-derived xenograft models for the full range of possible thresholds from 0 to 1 in 0.01 increments, and if multiple sequential optimal thresholds yield the same maximum F1 score, the median of the optimal thresholds is used.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); and Ausubel, F. M., et al. (eds.), Current Protocols in Molecular Biology, John Wiley & Sons, New York (2010); for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline;
or making the final point of degeneration less debilitating.

The treatment or amelioration of symptoms can be based on objective or subjective parameters, including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., a cancer), or any combination thereof. The term “therapeutic effect” refers to the reduction, elimination, or prevention of the disease or condition, symptoms of the disease or condition, or side effects of the disease or condition in the subject.

As used herein, the terms “nucleic acid” or “polynucleic acid” refer to a polymer of nucleotide monomer units or “residues”, typically DNA or RNA. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

The studies disclosed herein provide a framework to non-invasively classify and predict molecular subtypes of SCLC using cfDNA nucleosome profiling. The assay addresses the lack of available tools to monitor therapeutic response and link treatments to phenotypes in SCLC.

Publications cited herein and the subject matter for which they are cited are hereby specifically incorporated by reference in their entireties.

### EXAMPLES

### Example 1

This example provides an approach for cancer phenotyping based on targeted sequencing of cell-free DNA (cfDNA) for small cell lung cancer (SCLC). A targeted capture panel that identifies chromatin organization signature at 1535 transcription factors (TFs) and 13,240 gene transcription start sites and detects exonic mutations in 842 genes is demonstrated. Informative loci were identified, and the approach is shown to have utility for SCLC subtyping and monitoring the transformation from non-small cell lung cancer to SCLC.

### Methods

### Patient Recruitment and Tissue and Plasma Sample Procurement

### Human Subjects Research

### PDX Model Generation

SCLC and NSCLC PDX models were generated from circulating tumor cells or tumor tissue as previously described and propagated according to methods well known in the art.

### Blood and Plasma Sample Processing

From patients, peripheral blood was collected in K₂EDTA tubes. From NSG mice harboring flank tumors of lung cancer xenograft models, blood was collected by cardiac puncture. Fresh blood was pooled for up to five mice harboring the same model when available. Blood was then centrifuged at 2,500×g for 10 minutes at room temperature and plasma supernatant was removed by pipetting. At this step, for some patient samples, buffy coat was aspirated and stored at −80° C. Plasma was then centrifuged at 13,200×g for 10 minutes at room temperature to remove any residual debris, and supernatant was collected. Both plasma and buffy coat were stored at −80° C. until DNA isolation was performed.

### Tissue Transcriptome and Immunohistochemistry (IHC) Analysis

### RNAseq of PDX and Patient Tumor Samples

RNAseq of tumor tissue from PDX models was performed as follows. RNA was extracted from cell pellets or tumor samples using TRIzol™ reagent. RNAseq libraries were generated from 500 ng of RNA using the NEBNext® Ultra™ RNA Library Prep Kit for Illumina (New England Biolabs E7530L). Sequencing was performed using an Illumina HiSeq 2500 instrument in 50 bp single end read configuration. Fastq files were filtered to exclude reads that did not pass Illumina's base call quality threshold. STAR v2.7.1 (35) with 2-pass mapping was used to align reads to mouse genome build mm10 and GENCODE gene annotation M23 (See gencodegenes.org website; mouse). FastQC 0.11.9 (Babraham Bioinformatics website, projects section, FastQC) and RSeQC 3.0.0 (36) were used for QC including insert fragment size, read quality, read duplication rates, gene body coverage and read distribution in different genomic regions. FeatureCounts (37) in Subread 1.6.5 (37) was used to quantify gene-level expression by unstranded read counting. Bioconductor package edgeR 3.26.8 (38) was used to detect differential gene expression between sample groups. Genes with low expression were excluded using edgeR function filterByExpr with min.count=5 and min.total.count=15. The filtered expression matrix was normalized by the TMM method (39).

RNAseq transcript TPM values for the two SCLC cell lines (H211 and H526) that were included in the PDX were downloaded from the DepMap site (2019Q2 release) (41). RNAseq of patient tissue samples was performed as previously described (18).

### Immunohistochemistry of Patient Tumor Samples

Immunohistochemistry for ASCL1, NEUROD1, and POU2F3 was performed as previously described (21).

### SCLC Subtype and REST Activity Status Labeling

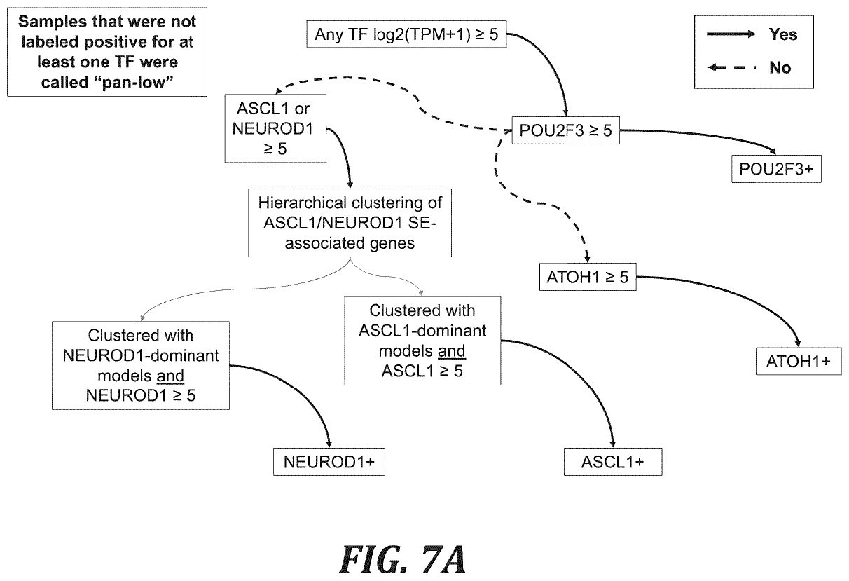

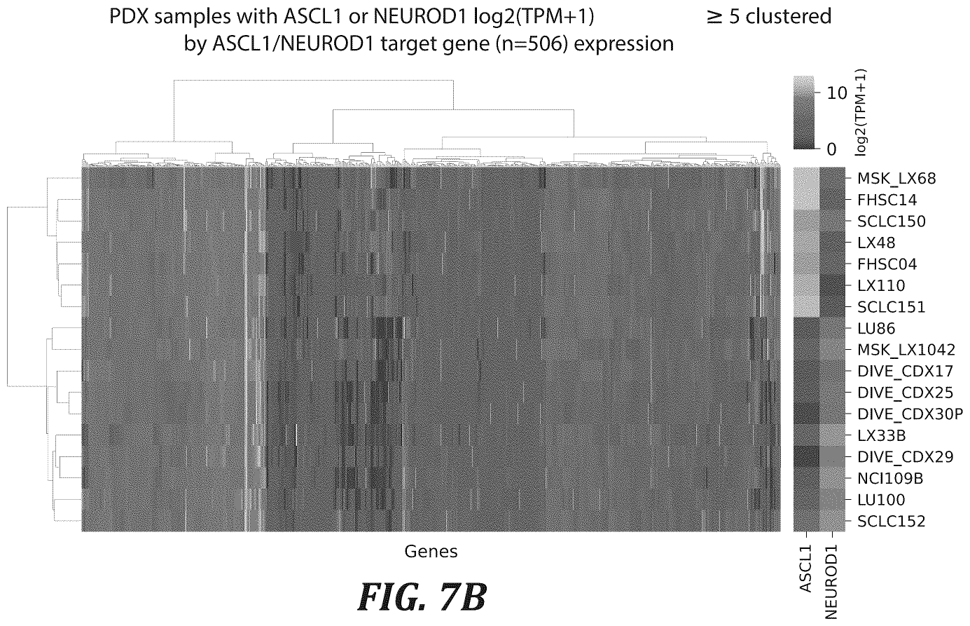

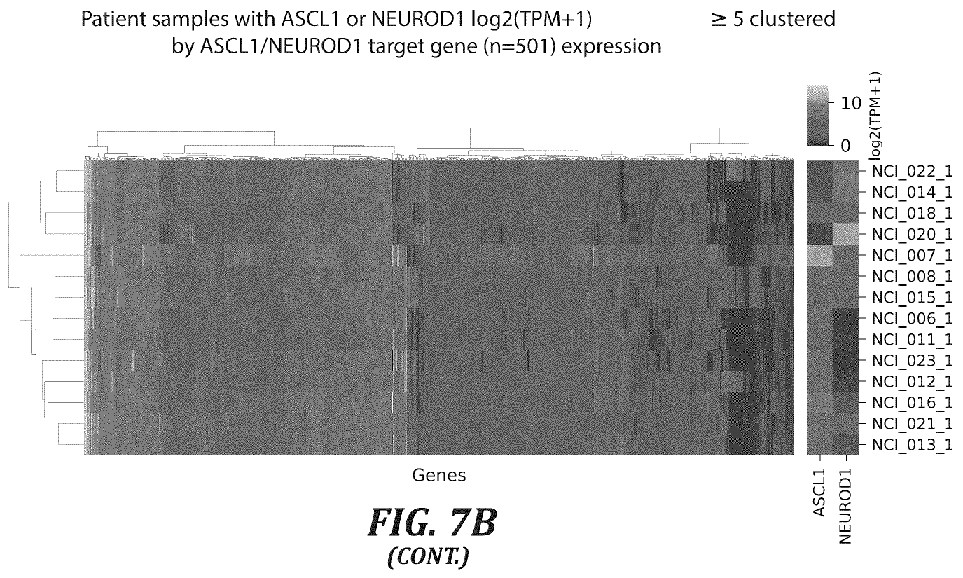

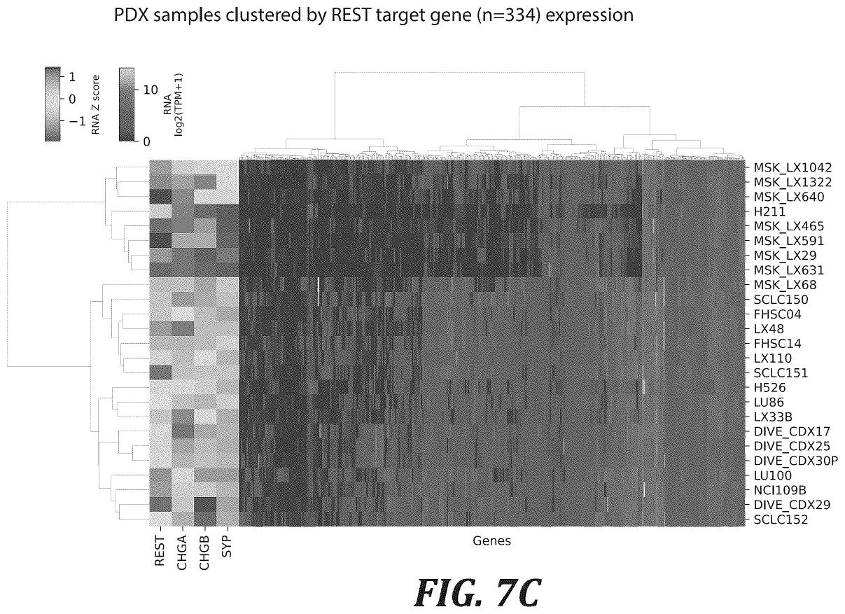

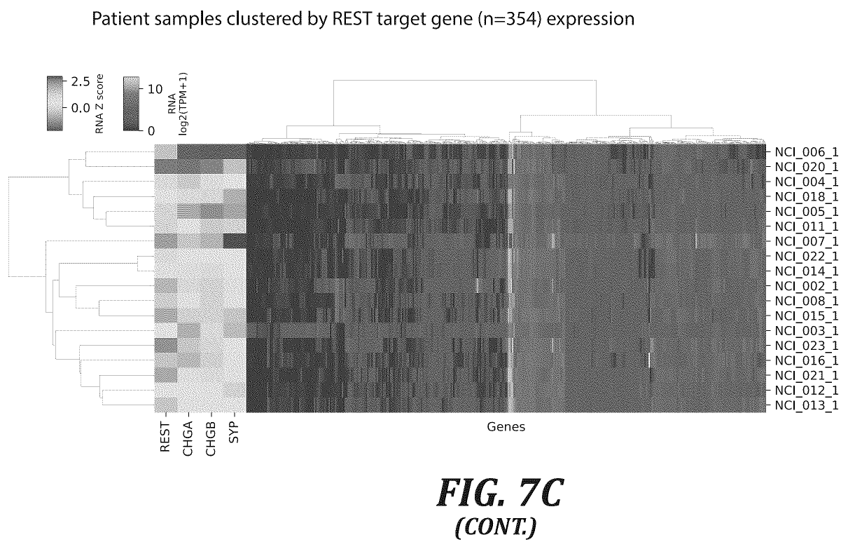

To facilitate interpretation of cfDNA nucleosome profiling efforts, a stepwise labeling scheme was developed for SCLC TF activity for SCLC and LCNEC PDX models and SCLC patient samples (FIG. 7A). ASCL1, ATOH1, NEUROD1, and POU2F3 activity in samples were assigned with available RNAseq data as follows. Transcript levels are considered in units of log₂(TPM+1). Samples with all TF transcripts≤5 were labeled “pan-low.” Samples with POU2F3 transcript>5 were labeled POU2F3-active. The remaining samples were examined for ASCL1, NEUROD1, and ATOH1 activity. Those with ATOH1 transcript>5 were labeled ATOH1-active. Because ASCL1 and NEUROD1 can be co-expressed, those with ASCL1 and/or NEUROD1 transcript>5 are subjected to unsupervised hierarchical clustering of the transcript levels of a group of 526 ASCL1 and NEUROD1-associated genes (62), resulting in two main clusters with divergent ASCL1 vs NEUROD1 expression; samples were labeled as ASCL1-active or NEUROD1-active based on relative expression of ASCL1 and NEUROD1 in the resulting clusters (FIG. 7B). Clustering was performed with the linkage method of the scipy software package (v1.7.1) with method=“ward” and metric=“euclidean.” For samples with IHC data, to assign ASCL1, NEUROD1, and POU2F3 activity, TF activity for ASCL1, NEUROD1, and POU2F3 was classified as positive, focal, or negative as described above. Subtype labels were generated as the union of all TFs that were considered active (including positive or focal for patient samples with IHC only). For example, a sample that was determined to be ATOH1-active and NEUROD1-active (a common occurrence) was given the subtype ATOH1+NEUROD1.

REST is a transcriptional repressor that can be expressed as an inactive splice variant (63), limiting the use of REST transcript level alone to determine REST activity. Therefore, tissue levels of REST target genes were examined, using the 383 genes in the “REST ENCODE” gene set obtained from the “ENCODE_and_ChEA_Consensus_TFs_from_ChIP-X” file hosted by the EnrichR pathway enrichment tool (64). REST activity was labeled based on sample grouping after unsupervised hierarchical clustering (as above) of samples by REST target gene transcript levels (FIG. 7C). In PDX models, REST-positive samples, including NSCLC samples, formed a clear distinct cluster at the first fork in the dendrogram (FIG. 7C). In patient samples, the first fork in the dendrogram did not separate REST positive from REST-negative samples; however, two samples (NCI_006 and NCI_020) clustered together and were notable for having the highest REST transcript levels and very low levels of the neuroendocrine genes CHGA, CHGB, and SYP(FIG. 7C). Therefore, these two patient samples were labeled REST positive.

### Targeted Capture Panel Design

### Gene Selection

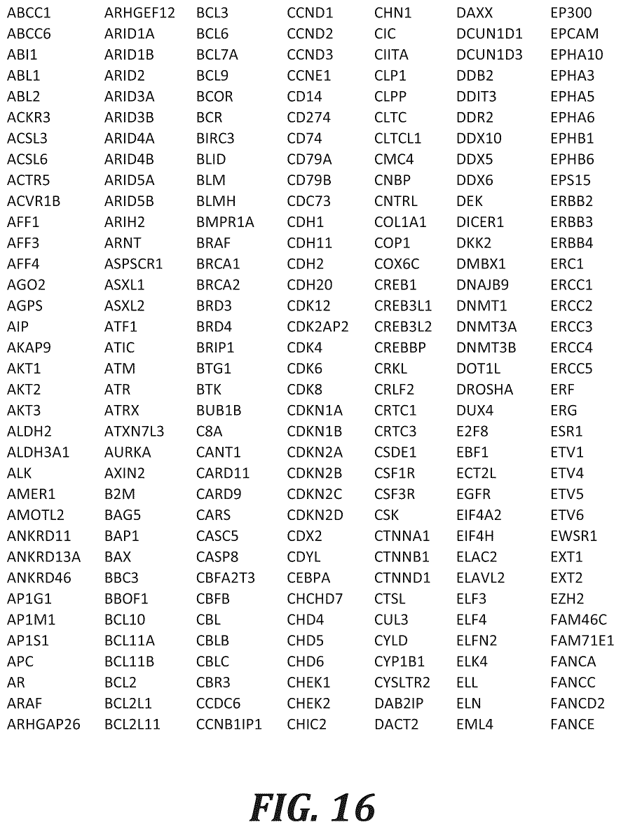

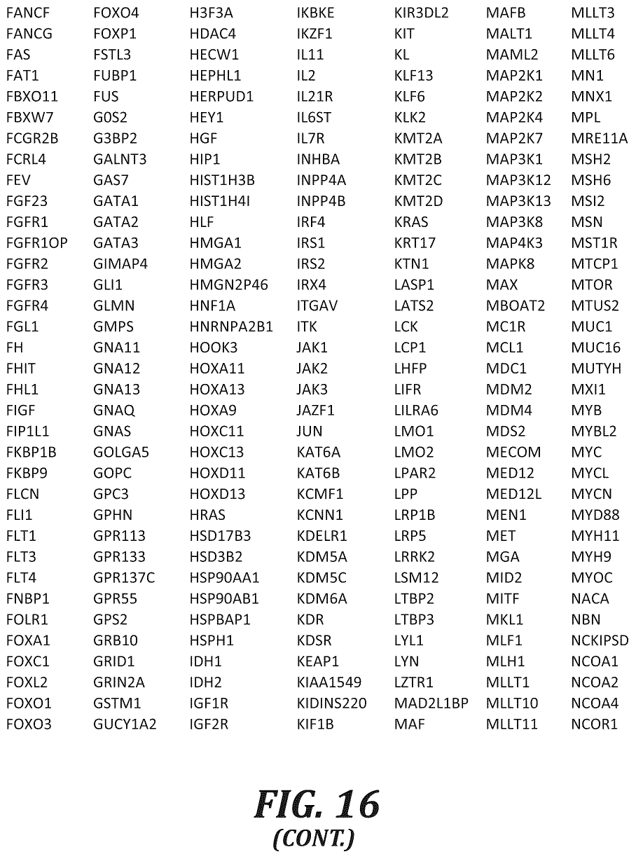

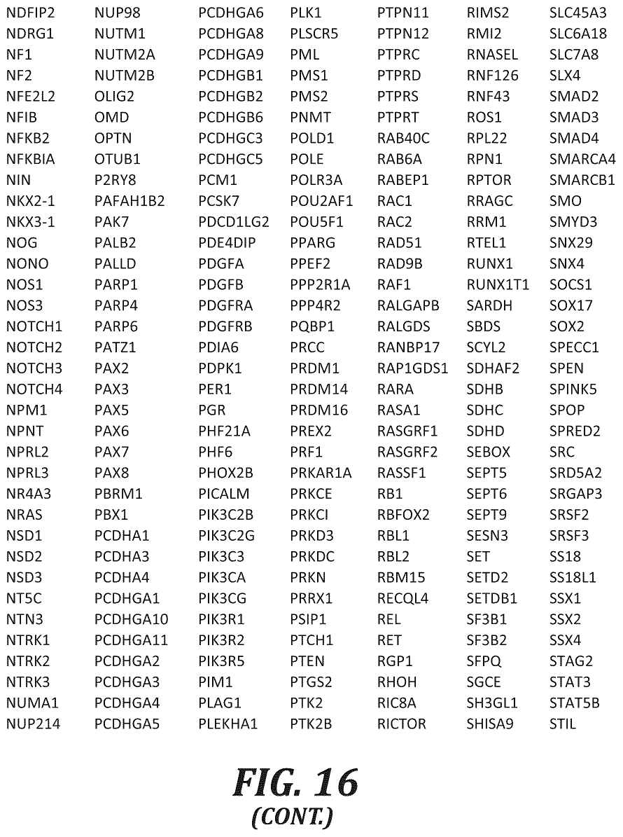

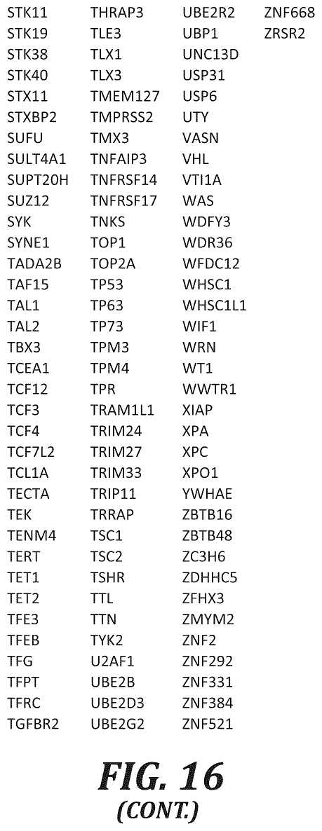

A panel of 842 genes was assembled manually using the following sources: (1) genes that are frequently mutated in SCLC according to publicly available databases (cBioPortal, Project GENIE), (2) genes with established or putative roles in SCLC biology according to literature review, and (3) genes that have been implicated in functional screens performed in the MacPherson lab. A full list of targeted genes is described in FIG. 16.

### Transcription Factor Binding Site Selection

Curated TFBS lists were generated individually for the SCLC TFs ASCL1, NEUROD1, POU2F3, and REST as follows. For ASCL1 and NEUROD1, ChIP seq peaks obtained for three ASCL1-expressing SCLC cell lines and two NEUROD1-expressing lines were obtained from published data (46). Peaks from all cell lines expressing the same factor (n=3 for ASCL1 and n=2 for NEUROD1) were intersected using “bedtools intersect” to create a stringent list of putative binding sites for each factor. Only putative binding sites at least 20 bp in length after intersection of lists of peaks across cell lines were retained. TF specific upregulated genes were obtained from the same publication (46). Putative binding sites were defined as associated with a differentially expressed gene if they were either within 10 kb of a differentially expressed gene TSS, or if the closest gene TSS on the same chromosome was differentially expressed (regardless of distance), and only differentially expressed gene-associated putative binding sites were retained.

For POU2F3, a ChIP-seq bedgraph file from the POU2F3-expressing SCLC cell line NCIH1048 was obtained from published data (59). Peak calling was performed using the bdgpeakcall function of the MACS2 package with the following flag string: “-c 2 -1 20 -g 20.” TF-specific upregulated genes were obtained from the same publication (59), and putative POU2F3 binding sites were annotated as being associated with a differentially expressed gene and filtered as described above for ASCL1 and NEUROD1.

For REST, putative binding sites were defined as peaks observed in at least five experiments in the GTRD human ChIP-seq database (65). SCLC cell line gene expression data (CCLE/DepMap 2019 Q2 dataset) (41) was then mined to create a coarse list of REST-associated differentially expressed genes as follows. Five REST-high SCLC cell lines (NCIH2286, NCIH1048, SW1271, DMS114, and NCIH1339) and five REST-low lines (NCIH2227, NCIH2171, NCIH1930, NCIH1092, and NCIH1836) were identified. An unpaired Welch's t-test of the transcript level estimates of each gene was then performed comparing these two groups of cell lines. REST-associated differentially expressed genes were defined as the 250 genes that were most significantly more highly expressed in REST high cell lines. Putative REST binding sites were then annotated as being associated with a differentially expressed gene and filtered as described above for ASCL1, NEUROD1, and POU2F3.

### Transcription Start Site (TSS) Selection

A list of human TSS from the Ensembl v97 annotation (corresponding to human genome version GRCh38.p12) was downloaded from Ensembl using the BioMart tool. Gene annotations from the Gencode v31 “Basic” gene annotation were downloaded from the University of California Santa Cruz (UCSC) genome browser. These two annotations were then merged based on the Ensembl “Transcript stable ID” field. The following filters were then applied. TSSs residing on alternative contigs or chromosome Y were excluded. TSSs corresponding to transcripts not labeled “protein_coding” or “retained_intron” were removed. TSSs were then required to have at least one of the following properties: (1) transcript support level equal to 1 according to Gencode v31, (2) a single exon gene, (3) association with the same genes that were defined as SCLC TF-associated when designing the TFBS component of the panel, or (4) presence in the MSigDB v7.0 Hallmark pathways list (66, 67). This resulted in a list of 36,379 TSSs corresponding to 18,030 unique genes. Sites were padded by 100 bp upstream and 260 bp downstream and these intervals were submitted to commercial vendor Twist Bioscience for custom capture panel design. The resulting probe set adequately targeted (defined as continuous probe coverage from 90 bp upstream to 245 bp downstream of the TSS) a total of 35,917 TSSs corresponding to 17,921 genes.

### Panel Synthesis

Desired genes and genomic regions surrounding TFBSs and TSSs of interest, as defined above, were submitted to a commercial vendor (Twist Bioscience) for custom targeted panel design and synthesis. Coding sequence and TFBS regions were targeted together in one 4.5 Mb panel. TSS regions were targeted in a second 9.2 Mb panel.

### DNA Isolation and Sequencing Library Construction

### DNA Isolation

Genomic DNA was extracted from buffy coat using the QIAamp DNA Blood Mini Kit (QIAGEN) according to manufacturer's instructions. Cell-free DNA was extracted from 0.5 to 4 mL of plasma using the QIAamp MinElute ccfDNA Mini Kit (QIAGEN) or the QIAamp Circulating Nucleic Acid Kit (QIAGEN) according to manufacturer's instructions. After isolation, DNA concentration was measured using the Qubit™ dsDNA HS kit (ThermoFisher Scientific) and size distribution was assessed using the Bioanalyzer instrument and DNA 1000 or HS DNA 1000 kit (5067-1504 or 5067-4626, Agilent).

### Whole Genome Sequencing Library Construction

Plasma cfDNA sequencing libraries were constructed using the NEBNext® Ultra™ II DNA Library Prep Kit for Illumina (New England Biolabs), or the xGen™ cfDNA & FFPE DNA Library Prep MC Kit with unique dual index PCR primers (Integrated DNA Technologies), according to manufacturer's instructions. DNA concentration and size distribution were again measured using the Qubit and Bioanalyzer systems (see above). Buffy coat genomic DNA was fragmented mechanically to a goal modal fragment size of approximately 250 nucleotides using sonication (Bioruptor® sonication instrument; Diagenode) with the following parameters: low, 30 seconds on and 90 seconds off, at 4° C., for 30 minutes. Individual samples were subjected to additional fragmentation as needed based on size distribution analysis. All buffy coat genomic DNA libraries were constructed using the xGen™ cfDNA and FFPE DNA Library Prep MC Kit with unique dual index PCR primers (Integrated DNA Technologies).

### Hybridization Enrichment

Whole genome sequencing (WGS) libraries were pooled and hybridized to one or both targeted panels according to manufacturer's instructions (Twist Bioscience) with the following modifications. WGS libraries were pooled in groups of between 8 and 26 libraries prior to capture. Equal DNA mass of each library was pooled, ranging from approximately 140 to 187.5 ng per library, where less than 187.5 ng of each library was used so as not to exceed a maximum total DNA mass in the pool of 4 μg. For cfDNA libraries, one of two capture strategies was used. Prior to establishing the functionality of the TSS panel, cfDNA libraries were captured using the two panels separately in parallel, and sequencing data were pooled in silico. Once the functionality of the TSS panel had been established in preliminary data, cfDNA libraries were captured using both panels in a single capture reaction. Buffy coat genomic DNA libraries were enriched using only the coding sequence and TFBS-targeted panel.

### Sequencing

All libraries were sequenced on Illumina instruments (HiSeq 2500 or NovaSeq) in a paired end configuration with individual read lengths ranging from 50 to 150 bp.

### Initial Sequencing Data Analysis

### Alignment and Removal of Reads Aligning to the Mouse Genome

For libraries constructed with the IDT xGen kit, which adds a variable sequence during the adapter ligation step that then serves as a unique molecular identifier (UMI), the UMI was extracted from the sequence read and retained as a bam file flag using the ExtractUmisFromBam command from the fgbio toolkit (version 1.3.0). Reference genomes for human (NCBI GRCh38) and mouse (NCBI GRCm38) were downloaded from the Illumina iGenomes website (See the Illumina website; sequencing software; igenome). For all libraries, sequence reads were then aligned using bwa-mem (version 0.7.17) to a concatenated human and mouse reference genome consisting of hg38 and mm10. Read pairs in which both reads were aligned to the human genome were retained for further processing (68). The retained reads were then realigned to the human genome using bwa mem. Next, duplicates were marked using the MarkDuplicates function from the Picard toolkit (version 2.18.29). For libraries constructed with the IDT xGen kit, duplicate marking was UMI-aware. Next, base qualities were recalibrated using the BaseRecalibrator workflow from GATK (version 4.1.8.1). Sorting steps throughout were performed with “samtools sort” (version 1.11) or GATK SortSam. Finally, sequencing metrics were calculated using the CollectAlignmentSummaryMetrics, CollectInsertSizeMetrics, and CollectHsMetrics functions from the Picard toolkit (version 2.25.0).

Tumor Fraction Estimation Using ichorCNA

The ichorCNA package (script version date Jan. 6, 2020) was applied to low coverage sequencing data with default settings. The tumor fraction prediction that was predicted as most likely was then used as the estimated tumor fraction for each sample in subsequent analyses.

Variant Calling from Plasma cfDNA

Plasma samples corresponding to patients for whom buffy coat (PBMC) sample was also available were subjected to variant calling. Buffy coat libraries were available for 46 independent patients. Of the 46 patients, 43 had SCLC and 3 had non-malignant lung nodules. Variant calling was limited to samples from patients with SCLC. Restricting to the 43 patients with SCLC, there were 40 cfDNA targeted libraries corresponding to 39 distinct patients that passed the QC filters described previously. Targeted sequencing data from cfDNA and buffy coat samples were processed using the variant callers Mutect2 (of the GATK package), Manta/Strelka (version 1.6.0 and 2.9.9, respectively), and CNVkit (version 0.9.7) as follows.

Single nucleotide variants (SNVs) and small insertions and deletions (indels) were called using the Mutect2 pipeline as follows. A “panel of normal” was generated from the five cfDNA libraries corresponding to patients with non-malignant lung nodule biopsy results (filtering of libraries due to lower coverage was performed after this step). Mutect2 was then run with a premade “germline-resource” file that was downloaded from the GATK website (“af-only-gnomad.hg38.vcf.gz”) as well as the custom “panel of normal” vcf in a germline-matched configuration (with “-normal” flag set). FilterMutectCalls was then run with default settings and only variants passing filtering were retained.

In parallel, single nucleotide variants (SNVs) and small indels were called using the Manta/Strelka pipeline. Manta was run using default settings in a germline matched configuration to generate a list of candidate indels. Strelka was then run with default settings in a germline matched configuration including the preliminary indel list generated by Manta.

Variants called by the Mutect2 and Manta/Strelka pipelines were then annotated using the Funcotator tool in GATK with default settings and the prepackaged data source version v1.6.20190124s (downloaded from the GATK website). SNV and small indel calls were then additionally filtered as follows. Only calls that were made and passed default filters in both Mutect2 and Manta/Strelka were retained. Furthermore, variants were only analyzed that were predicted to change protein sequence or affect a splice site.

Each sample was also inspected for possible mis-assignment to the germline sample by examining whether predicted somatic variants tended to occur in a commonly used germline database (gnomAD). Indeed, for a single sample (FH_083_1), a substantial fraction of predicted somatic variants were present in the gnomAD database. This sample was therefore excluded from the final analysis.

To call larger copy number variants (CNVs), the CNVkit pipeline was used as follows. The normal sample reference was constructed from all the available buffy coat targeted libraries (n=46). Buffy coat libraries corresponding to four SCLC patient cfDNA libraries were included that had failed QC and three non-malignant control patient cfDNA libraries as it was reasoned that the buffy coat libraries from these individuals could still provide additional valuable data for normal reference construction. The accessible region of the genome was defined using the “cnvkit.py access” command as the hg38 genome sequence excluding the TSS component of the targeted panel (with each TSS-containing targeted region padded by 300 bp). CNV calling was then performed using the “batch” command. CNV calls were filtered and labeled as follows:
Intergenic variants and variants involving chrY were removed
Variants with absolute value of log₂ fold-change of less than 1 were removed
Variants involving the gene DUX4 were removed, as DUX4 is normally present in variable copy number in a genomic macrosatellite region and interpretation of copy number change predictions are therefore substantially more challenging for this gene Genotyping results were visualized with CoMut (69).

### Nucleosome Occupancy Profile Determination Using the Griffin Platform

### Griffin Implementation for Targeted Panel Sequencing Data

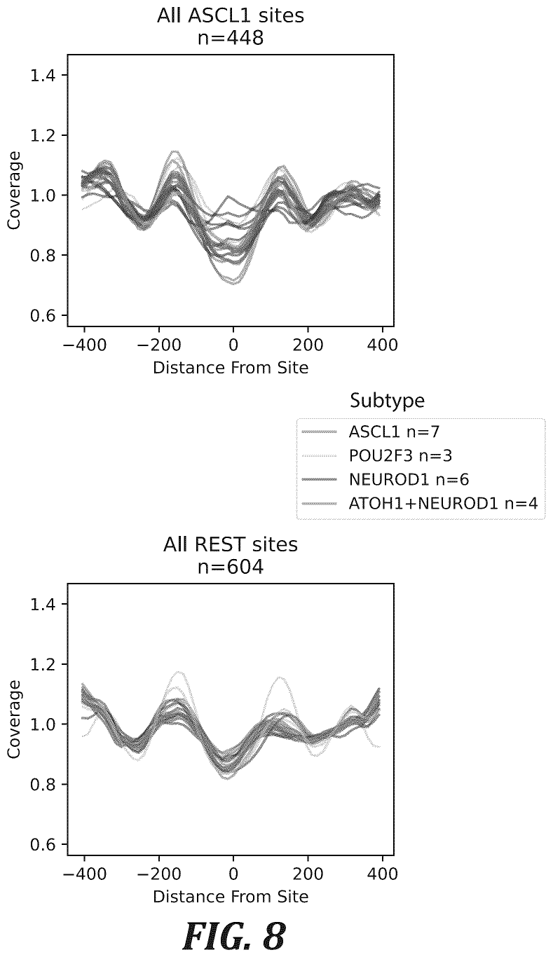

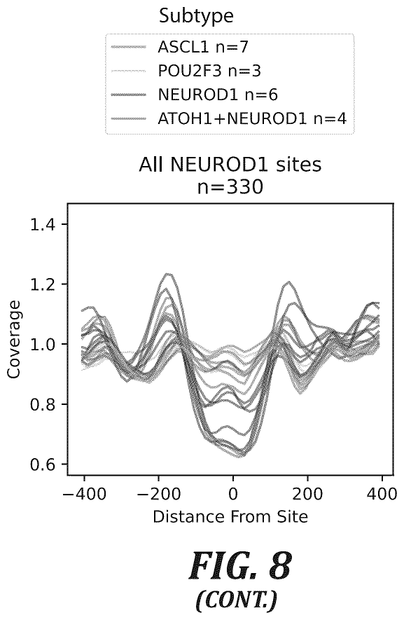

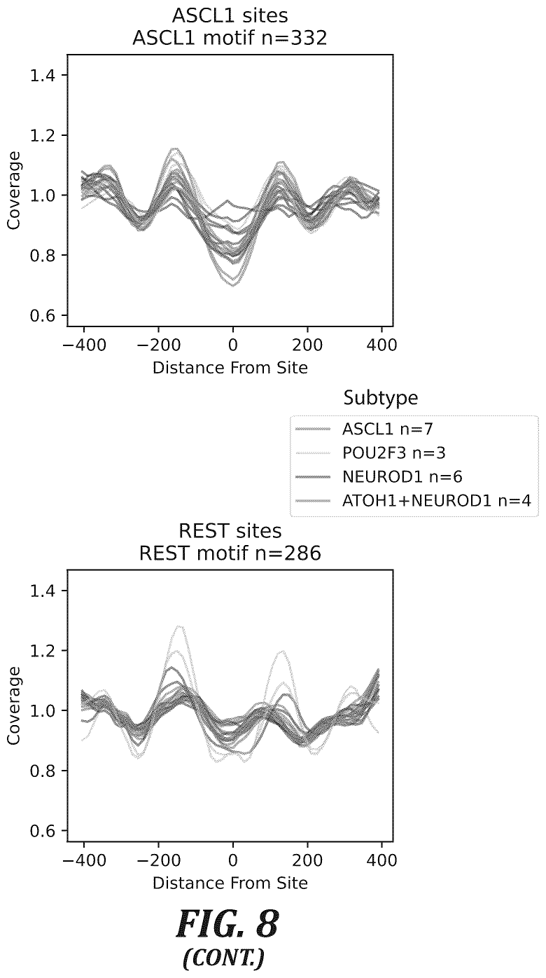

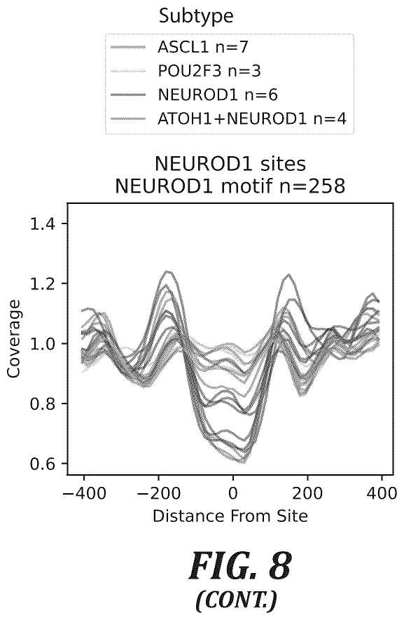

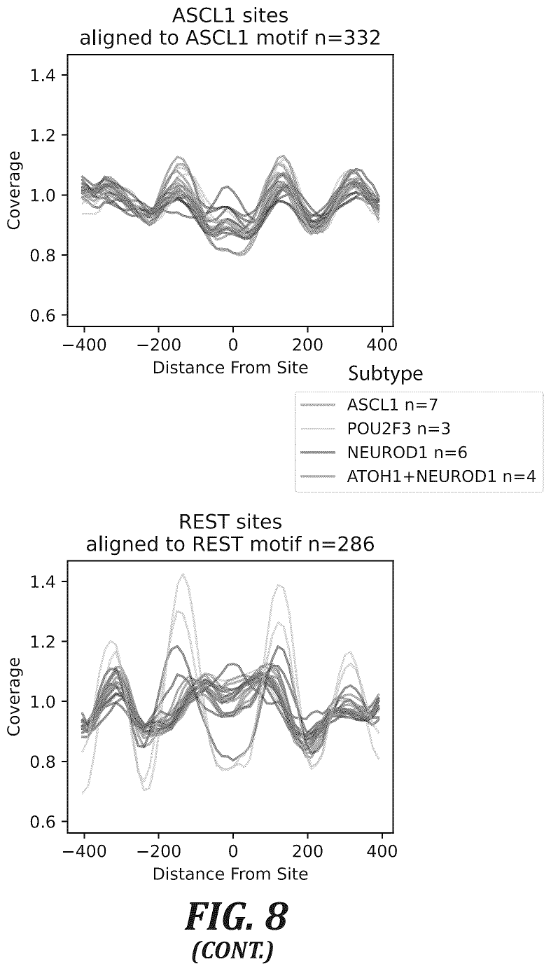

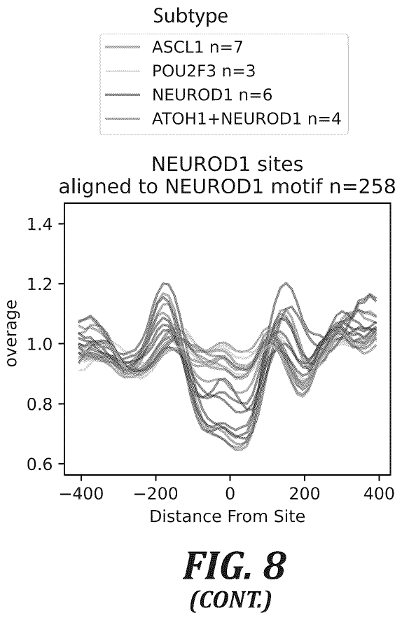

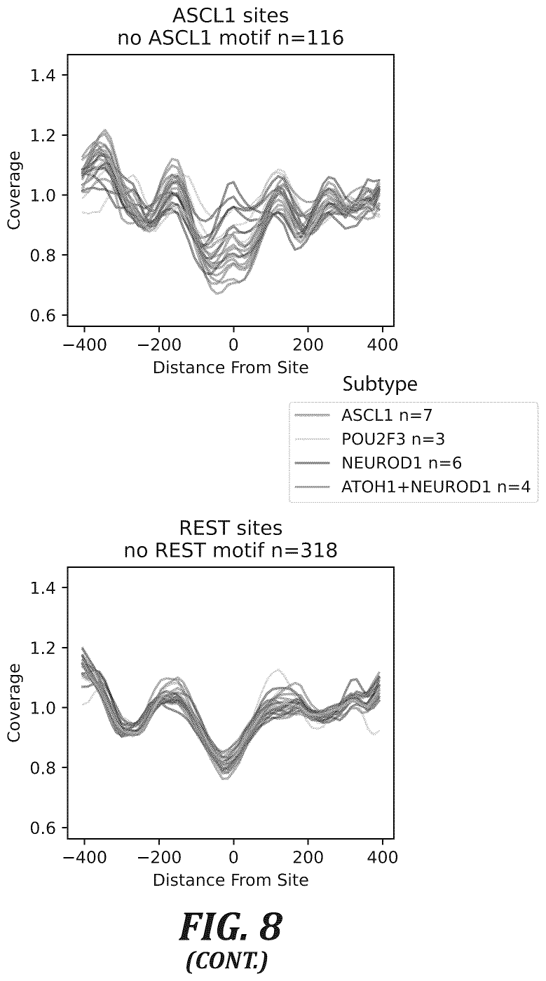

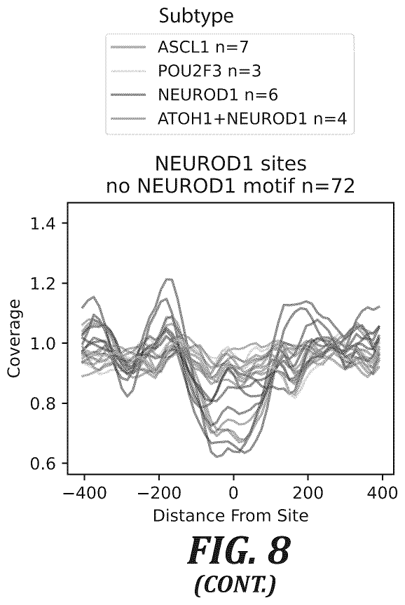

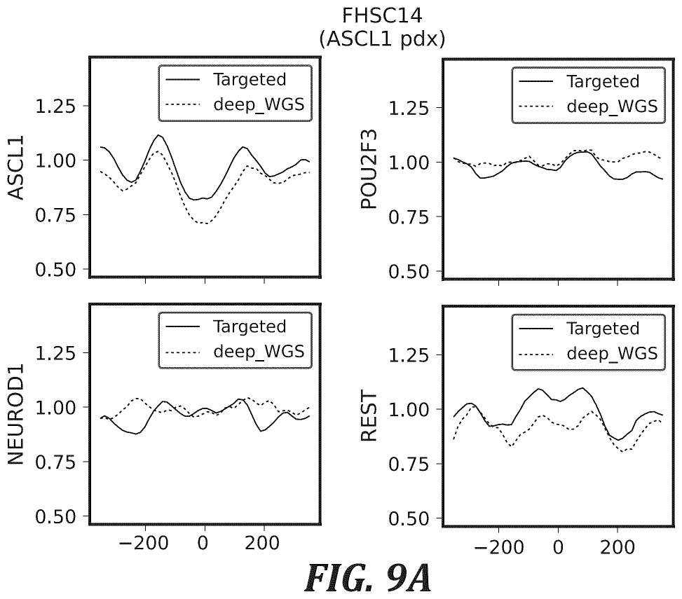

Griffin nucleosome occupancy profiling was performed as previously described (57) with some minor modifications as follows. First, when running GC correction, only positions that were covered by probes in the targeted panel were considered when counting reads and GC content. Second, separate GC corrections were performed for regions targeted by the TSS component versus the TFBS and coding sequence component of the panel. Third, 50 bp read mappability was used when identifying mappable positions (the shortest read lengths in this study were 50 bp). Fourth, when performing nucleosome profiling, coverage profiles were calculated in the window±495 bp from a targeted TFBS or a −90 to +240 from targeted TSS. Additionally, sites that were not fully covered by the targeted panel were excluded from further analysis. Coverage outliers were retained (i.e., “exclude_outliers=False” used) and individual sites were normalized to a mean coverage of 1 (CNA_normalization=True). These filters resulted in estimation of nucleosome occupancy profiles for 1,853 TFBSs (448 ASCL1, 330 NEUROD1, 471 POU2F3, and 604 REST), and 32,717 TSSs corresponding to 16,160 genes. It was also examined whether in silico centering onto TF binding motifs at each TFBS prior to averaging influenced the shape of composite nucleosome profiles. Motifs in the targeted TFBSs were identified using motifmatcheR to search for instances of the HOCOMOCO motifs from CIS-BP (70) for REST, ASCL1, and NEUROD1. Coverage profiles were then aligned so that the motif (or the highest scoring motif if there was more than one) was in the center. Recentering of the window of interest to a consensus binding motif improved REST signal strength by visual inspection but did not substantially affect the other TFs (FIG. 8); therefore only motif-centered REST profiles (n=286 sites) were included in all subsequent analyses. This resulted in a final list of 1,535 TFBSs.

### Griffin Implementation for Deep Whole Genome Sequencing Data

For Griffin analysis of deep whole genome sequencing, analysis was also performed as previously described with minor modifications to the nucleosome profiling step. GC correction was performed as previously described using the entire genome and 100 bp read mappability. When performing nucleosome profiling, coverage profiles were calculated in a window±1000 bp and normalized by the coverage in a window±5000 bp. 100 bp mappability was used to omit coverage at low mappabilty positions within sites. Other parameters were the same as the targeted panel analysis as follows. Coverage outliers were retained (i.e., “exclude_outliers=FALSE”), individual sites were normalized to a mean coverage of 1 (CNA_normalization=True). REST sites were aligned with the same procedure as used for the targeted panel. This resulted in analysis of the same 1,853 TFBSs (1,535 TFBSs after REST re-centering) and 32,717 TSS that were analyzed with the targeted panel.

### Curation and Annotation of TSS for Interpretation of Nucleosome Occupancy Analyses

Assignment of One TSS to Each Gene with Available Expression Data

To facilitate gene expression prediction benchmarking and interpretation, CCLE transcript-specific gene expression data was used from the 2021Q1 CCLE data repository to select, for each gene, a single TSS associated with the most highly expressed transcript across SCLC cell lines. The TSS corresponding to the most highly expressed transcript was covered by our targeted panel for 15,615 genes. A TSS for 1,412 genes was also retained in which the TSS associated with the most highly expressed transcript was not covered by the targeted panel but a TSS corresponding to a similarly expressed transcript was. Finally, 34 genes in which only one TSS was covered by the targeted panel was also retained, even though that site did not correspond to the most highly expressed transcript in CCLE data. A total of 17,061 one-to-one TSS to gene mappings were therefore generated. This list was then intersected with TSS that were successfully analyzed by Griffin nucleosome occupancy profiling, genes for which transcript levels were estimated in all the PDX and patient samples in which RNAseq had been performed, and sites at which the entropy feature could be calculated. This resulted in a curated list of 13,240 gene:TSS pairings for subsequent analyses.

### Determination of Distance to the Nearest CpG Island

CpG island locations were downloaded from the UCSC table browser (table cpgIslandExt for UCSC genome version hg38, most recently updated on 3/13/2020). The distance from each TSS to the nearest CpG island was calculated using the “closest” function in the Bedtools package (v2.30.0). TSSs were labeled CpG proximal if the nearest CpG island was <1 kb away.

Calculation of Descriptive Features from Griffin Nucleosome Occupancy Profiles

### TFBS Central Amplitude

The central amplitude was calculated as the arithmetic difference between the mean coverage across two windows flanking the TFBS (−195 to −120 bp and 120 to 195 bp) and the mean coverage at the TFBS (−30 to 30 bp).

### TSS Amplitude

TSS amplitude was defined as the arithmetic difference between the coverage at the +120 position and the −45 position (relative to the TSS).

### TSS Fragment Length Entropy

To calculate the fragment length entropy at individual TSSs, the distribution of aligned fragment lengths in 5 bp bins was first tabulated. Aligned fragments were defined as fragments with mapping quality of at least 20 in both reads ranging in length from 15 to 500 bp that overlapped any of the region 90 bp upstream to 240 bp downstream of each TSS. The entropy of these binned length distributions was then calculated using the scipy.stats.entropy function. Entropy scores were then Z score normalized within each sample.

TFBS k-Means Clustering and Profile Shape Similarity Metric Calculation

At each TFBS, k-means clustering (k=2) of the nucleosome occupancy profiles of the PDX samples was performed. Therefore, clustering at each site was performed on 25 profiles for ASCL1, POU2F3, and REST sites, and on 21 profiles for NEUROD1 sites. Nucleosome occupancy profiles were trimmed to the central 720 bp to minimize the contribution of noisier signal at the edges of captured windows. Clustering was performed with the sklearn.cluster.KMeans module of the scikit-learn package with random_state=0. The adjusted Rand index was then calculated at each TFBS as an indicator of the extent to which variation in nucleosome occupancy profile at that site was related to tissue expression of the corresponding TF. PDX models were classified as positive or negative for a given TF as described above, and the adjusted Rand index of the clustering assignment was calculated using the sklearn.metrics.adjusted_rand_score module of the scikit-learn package. Sites with an adjusted Rand index of at least 0.5 were considered to be informative. The results of the clustering at each site was also used to calculate average nucleosome profiles within each of the two clusters. Average profiles were labeled as “high” or “low” expressing of the TF of interest based on mean relative expression of the TF in samples in each cluster. Next, for each sample at each site, the Euclidean distance between the occupancy profile observed and each of the two average profiles was calculated. Finally, the log₂ transformed ratio of the two Euclidean distance metrics was calculated, where the distance to the “low” expressing cluster was in the numerator and the distance to the “high” expressing cluster was in the denominator.

TSS k-Means Clustering and Profile Shape Similarity Metric Calculation

TSS k-means clustering and profile shape similarity metric calculation was performed as described above for individual TFBSs with the following modifications. Nucleosome occupancy profiles were trimmed to a −60 to +165 bp window relative to the TSS to minimize the contribution of noisier signal at the edges of captured windows. For each TSS of interest, k-means clustering was performed with k=2 of the nucleosome occupancy profiles in the PDX samples. For each site, it was also then tested whether the clustering yielded two distinct expression states by performing a Mann-Whitney U test of the log₂(TPM+1) expression values for the gene of interest in the samples in each of the two clusters. Sites were then filtered based on the Mann Whitney U test p values to identify sites where the coverage profiles significantly discriminated tissue gene expression in the PDX models. To minimize the inclusion of spuriously informative sites, it was also required that the difference in mean expression between the two groups of samples generated by clustering was at least four-fold. Finally, for each sample, the Euclidean distance of the coverage profile at that TSS to the mean coverage profile of each of the two clusters was also calculated, and a single metric of relative distance was calculated as the log₂ of the ratio of the distance of the coverage profile to the low expression cluster mean over the distance of the coverage profile to the high expression cluster mean.

Identification of Genes that are Correlated with Key SCLC TFs

SCLC cell line gene expression data from CCLE (2021 Q2 dataset) was analyzed for the correlation of individual gene expression values with the expression of the key SCLC TFs ASCL1, ATOH1, NEUROD1, and POU2F3. TF-associated genes were defined as genes with a Pearson correlation coefficient greater than 0.6 with a given TF. Identification of genes that are differentially expressed between NSCLC and SCLC cell lines.

NSCLC and SCLC cell line gene expression data from CCLE (2021 Q2 dataset) were analyzed for differential expression of genes between histologic categories. Genes were categorically labeled as expressed or not expressed based on a log₂(TPM+1) cutoff of 1. Differential expression status was tested using Chi-squared and genes with differential expression FDR<0.1 (Benjamini-Hochberg) were considered histology specific.

### Formulation of Predictive Models for Lung Cancer Histology and SCLC TF Activity Using Targeted Nucleosome Profiling

### Model Formulation

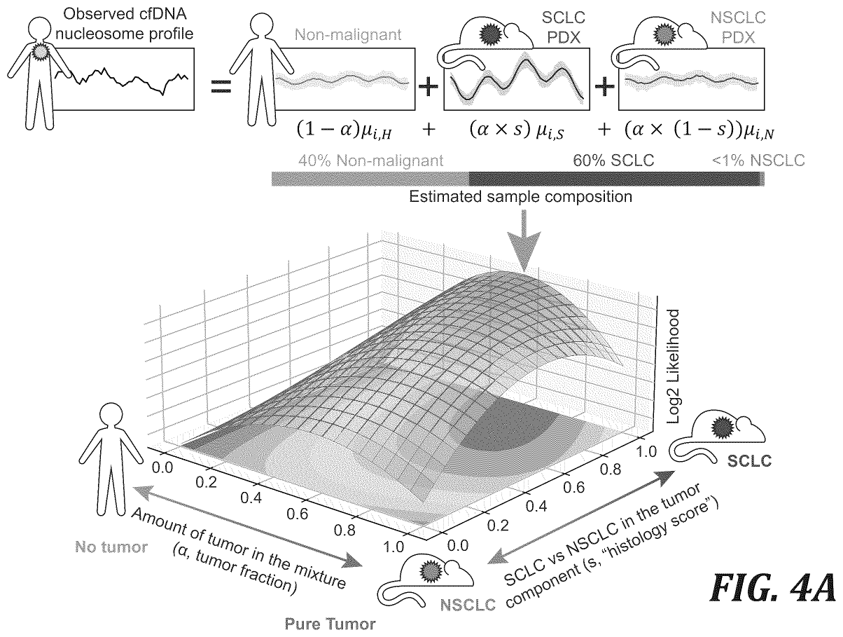

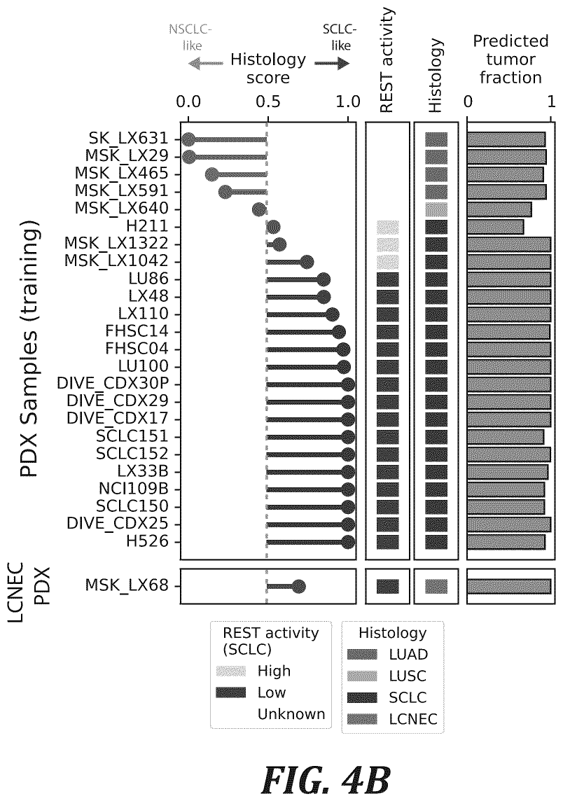

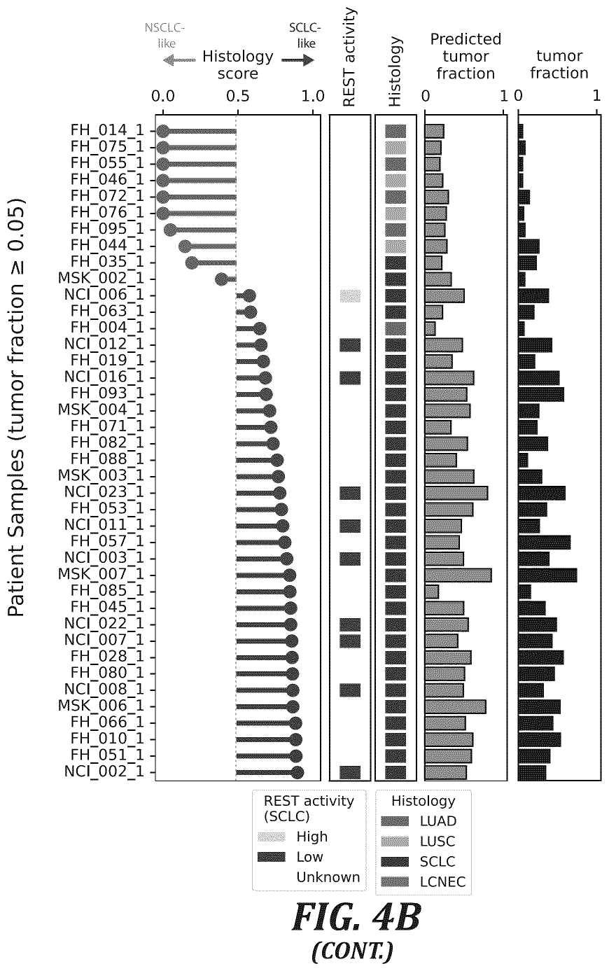

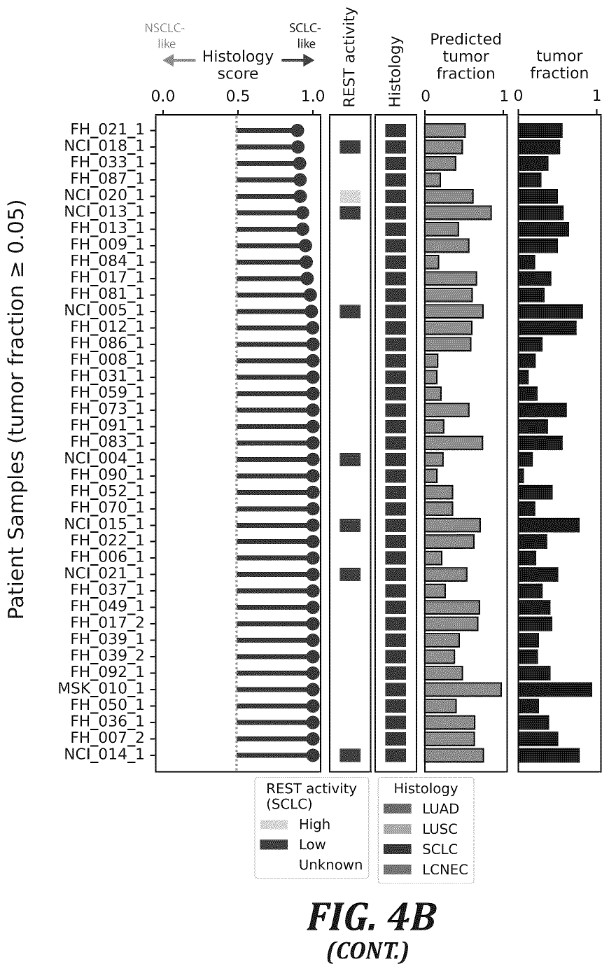

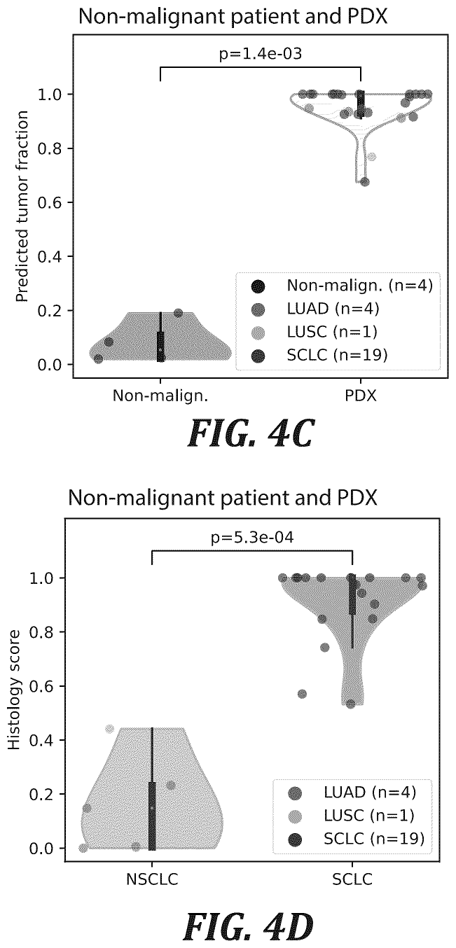

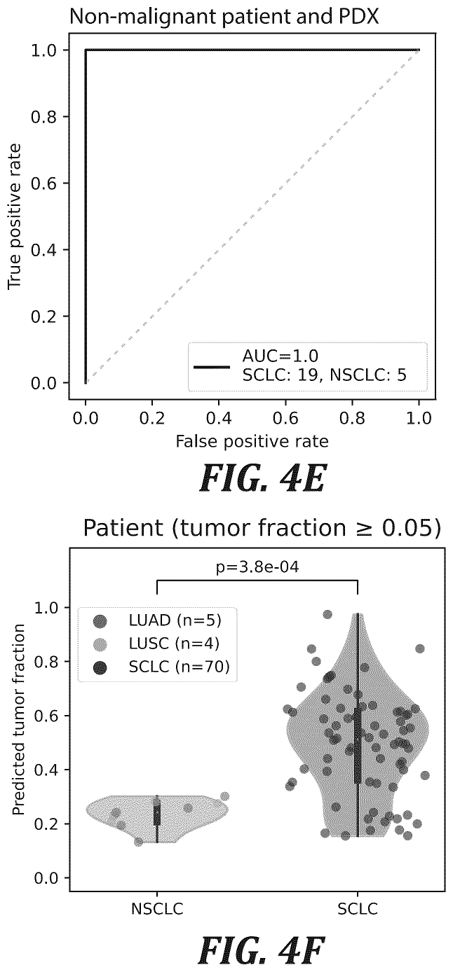

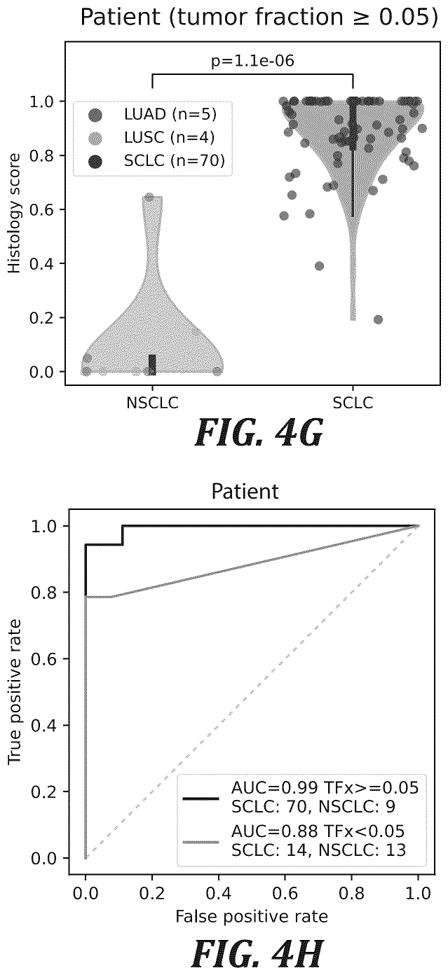

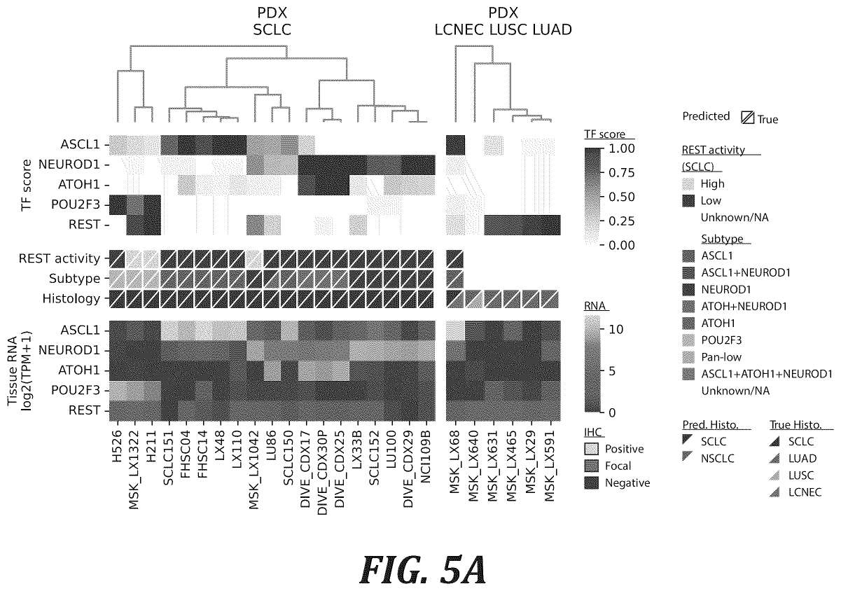

A probabilistic model was designed to estimate the composition of patient samples with respect to lung cancer histology and SCLC TF activity. Because cfDNA in cancer patients is a mixture of DNA derived from healthy and tumor cells, the cfDNA nucleosome relative coverage profiles observed in patients were modeled as a linear combination of the coverage profiles in cfDNA derived from “healthy” (non-malignant) cells only and the coverage profiles in cfDNA derived from tumor cells only. The tumor cell only component was also modeled as a linear combination of cfDNA coverage profiles derived from one of two phenotypic states of interest (e.g., NSCLC or SCLC, or TF-positive vs TF-negative) (see FIG. 4A). Specifically, for each genomic position “I” within the coverage profile, the expected coverage (μi) in a patient sample is a function of “a” which is the fraction of cfDNA that is tumor derived (“tumor fraction”) and “s” which is the fraction of tumor cfDNA that is derived from the phenotypic state. This relationship was modeled with the following equation:

$${μ}_{i} ( α , s ) = ( 1 - α ) {μ}_{i , H} + ( α \times s ) {μ}_{i , S} + ( α \times ( 1 - s ) ) {μ}_{i , N}$$

The expected coverage values for each component (μ_{i,H}, μ_{i,S} and μ_{i,N}) and μ_(i,N)) were determined by the mean coverage values observed in the data in patients without cancer (non-malignant) or in PDX samples. The 4 non-malignant patient samples were used to calculate the “healthy” coverage mean (μ_{i,H}). Phenotype-positive PDX samples were used to calculate μ_{i,S} and phenotype-negative samples were used to calculate μ_{i,N}. The standard deviation for each feature (ag) was calculated as the mean of the standard deviations from each of three states, as follows

$${σ}_{i} = \frac{{σ}_{i , H} + {σ}_{i , S} + {σ}_{i , N}}{3}$$

These relationships were then used to model the likelihood of a given tumor fraction (a) and subtype fraction (s) given the vector of features (coverage at each position, F_{0:T} observed in cfDNA from a particular sample.

$$ℒ ( α , s \vert {F}_{0 : T} ) ∝ {\prod }_{i = 0}^{T} 𝒩 ( {F}_{i} \vert {μ}_{i} ( α , s ) , {σ}_{i} )$$

The probability was calculated of each feature Fi being derived from a normal distribution with mean μᵢ(α,s) and standard deviation σᵢ and then take the product of those probabilities as an estimate of the relative likelihood of a given a and s giving rise to the observed signal. The “a” value and “s” value that have the highest likelihood of creating the observed data was then found. This was achieved by taking the −log₂ of the likelihood value and using the minimize function from scipy.optimize (version 1.7.1) to estimate “a” and “s” simultaneously. This framework was used to generate 6 models. The first predicted the histology (SCLC or NSCLC) and in this case “s” is referred to as the “histology score.” The other 5 models predicted TF activity in SCLC samples (ASCL1, NEUROD1, ATOH1, POU2F3, or REST) and for these models “s” is referred to as the “TF score.”

### Model Training and Features

Each predictive model was trained with samples that represented the phenotypic states of interest. For all models, all 4 non-malignant patient samples were used to estimate the mean feature values in the “healthy” component of cfDNA (μ_{i,H}). PDX models were used to estimate the mean feature values for a tumor with (μ_{i,S}) or without (μ_{i,N}) the phenotype of interest. The specific PDX models used depended on the phenotype being predicted. For the SCLC vs NSCLC model, SCLC PDX samples (n=19) were used to estimate the mean for the phenotype of interest (μ_{i,S}) and both LUAD (n=4) and LUSC (n=1) PDX samples to estimate the mean for the non-phenotype of interest (μ_{i,S}). The LCNEC PDX (n=1) was excluded from the training set due to shared features with both NSCLC and SCLC. For the TF activity predictors, only SCLC PDX samples were used in the training set. TF-positive models were used to calculate μ_{i,S} while TF-negative models were used to calculate μ_{i,S}. For preliminary testing, models were trained and tested on PDX and non-malignant patient samples using leave-one-out cross validation. For testing on SCLC and NSCLC patient samples, models were trained on the entire set of PDX and non-malignant patient samples.

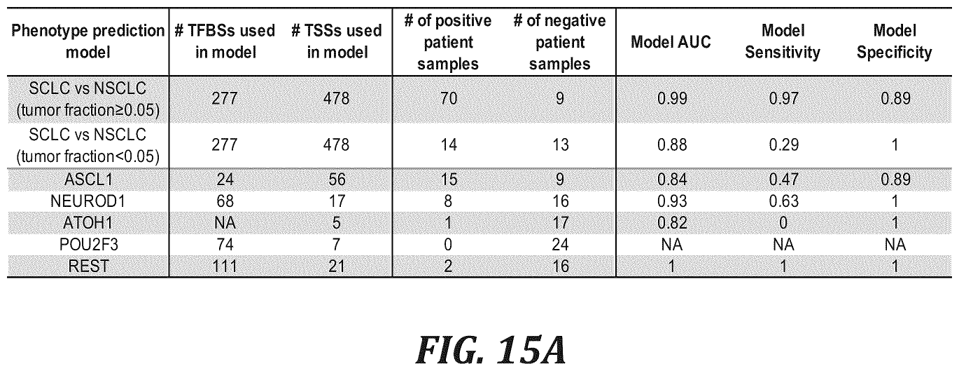

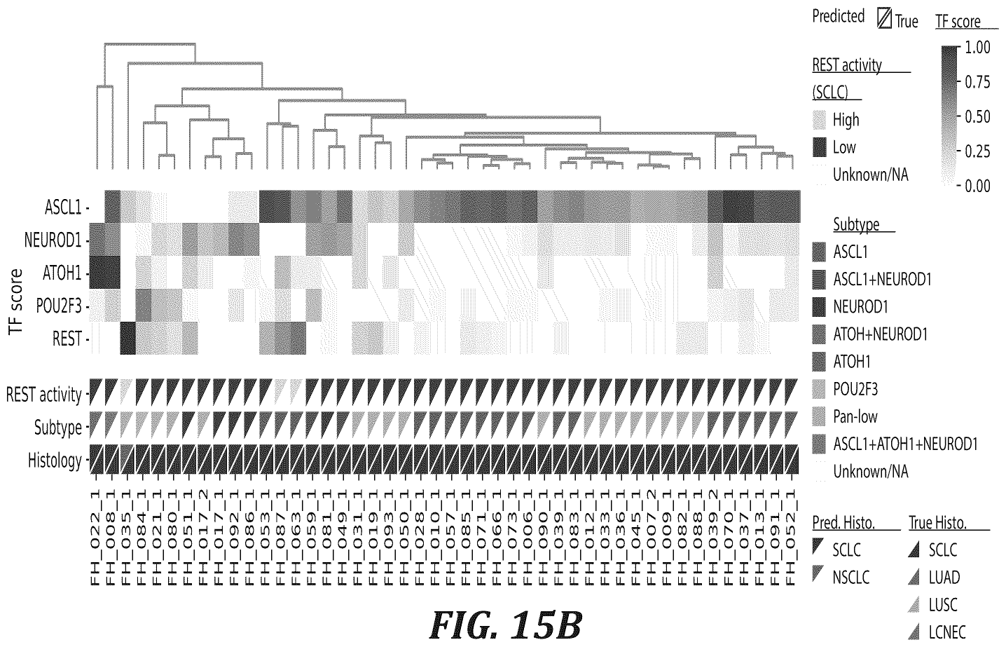

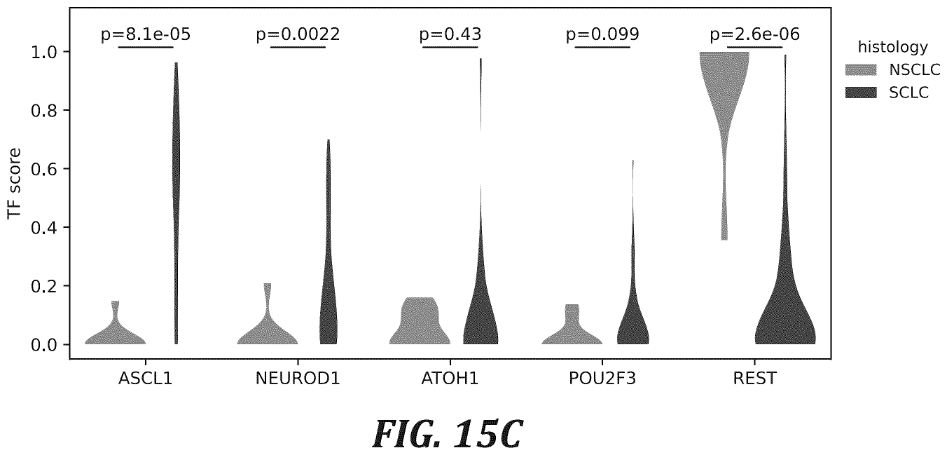

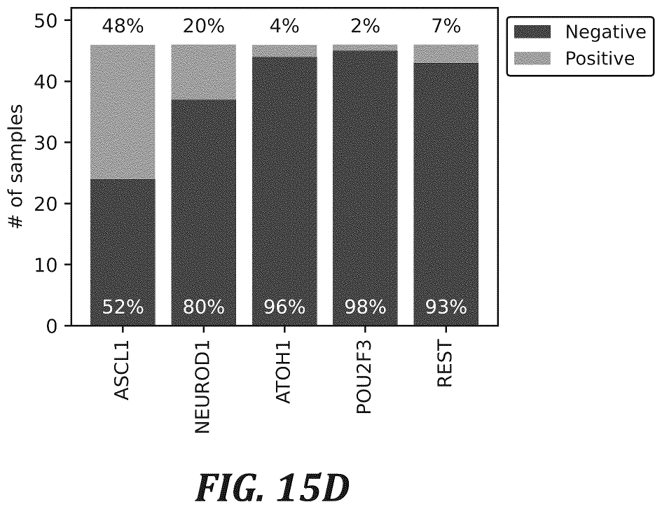

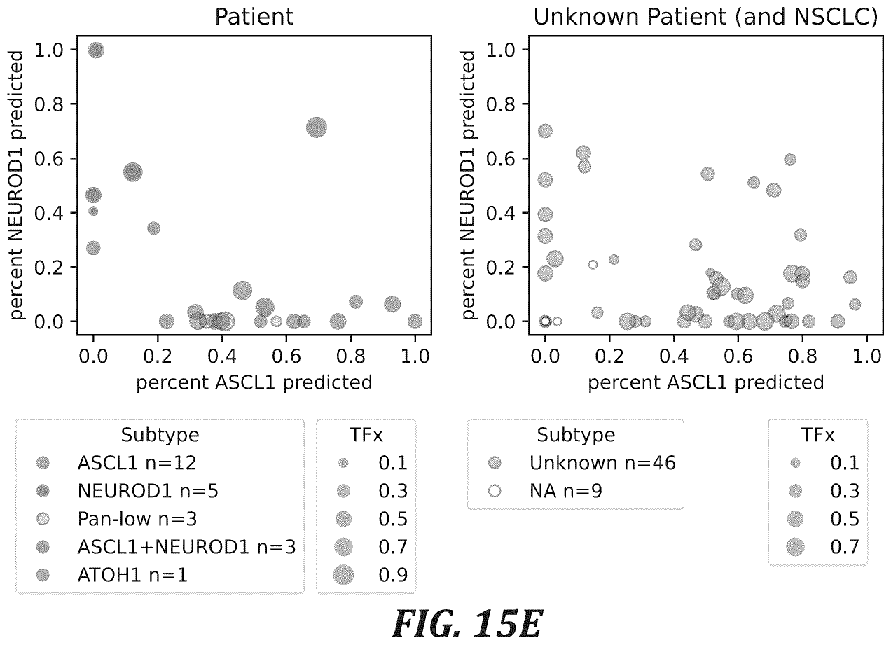

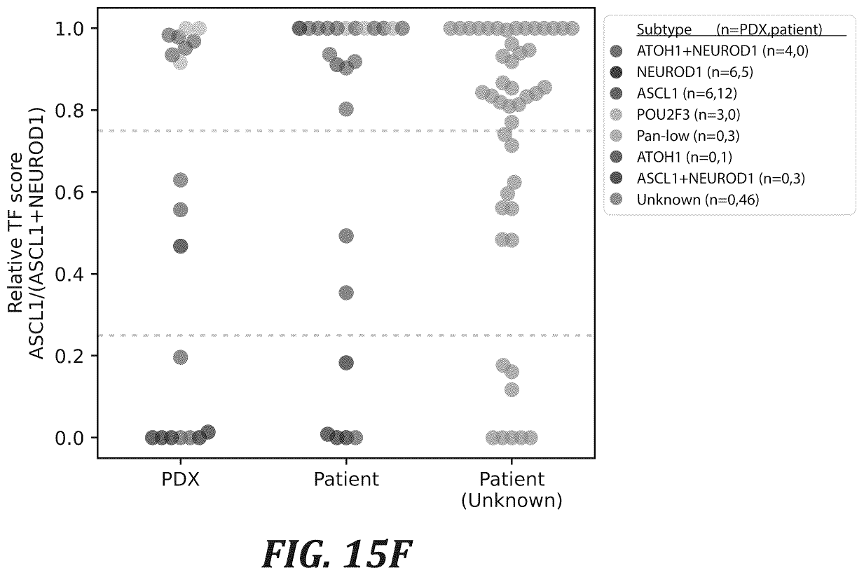

For each probabilistic model, a combination of TFBSs and TSSs that were informative and relevant to the phenotypes of interest was used (see FIG. 15A). Within each TFBS the Griffin relative coverage in 15 bp bins from −360 bp to 360 bp around the TFBS was used. This resulted in a total of 48 features per TFBS except for some REST sites as follows. REST sites had been re-centered to the nearest contained REST motif as described previously (see “Griffin implementation for targeted panel sequencing data”), which led to some sites with less than 360 bp of flanking targeted sequence on one or both sides. At these sites, non-targeted bins were excluded, and fewer than 48 features were used. Within each TSS the Griffin relative coverage in 15 bp bins from −60 bp to 165 bp around the TSS was used. This resulted in a total of 15 features per TSS. For the SCLC vs. NSCLC predictor, all 277 TFBS which were determined to be informative in the analyses described above were used (see “TFBS k-means clustering and” above) and 478 TSS which were determined to be informative and coarsely differentially expressed between SCLC and NSCLC cell lines as described above (see “TSS k-means clustering and profile shape similarity metric calculation” and “Identification of genes that are differentially expressed between NSCLC and SCLC cell lines” above). For the TF activity prediction, we again restricted to informative TFBSs associated with a given TF and informative TSSs associated with genes with expression correlated to SCLC TF expression in SCLC cell line RNAseq data (see “TFBS k-means clustering and profile shape similarity metric calculation”, “TSS k-means clustering and profile shape similarity metric calculation”, and “Identification of genes that are correlated with key SCLC TFs”) (FIG. 15A).

### Threshold Calculation

To select the optimal thresholds for the disclosed models, the F1 Score was calculated in the PDX models for the full range of possible thresholds from 0 to 1 in 0.01 increments. If multiple sequential optimal thresholds yielded the same maximum F1 score, the median of the optimal thresholds was used.

### Results

An Integrated cfDNA Sequencing Assay for Mutation Detection and Inference of Tumor TF Activity and Gene Expression

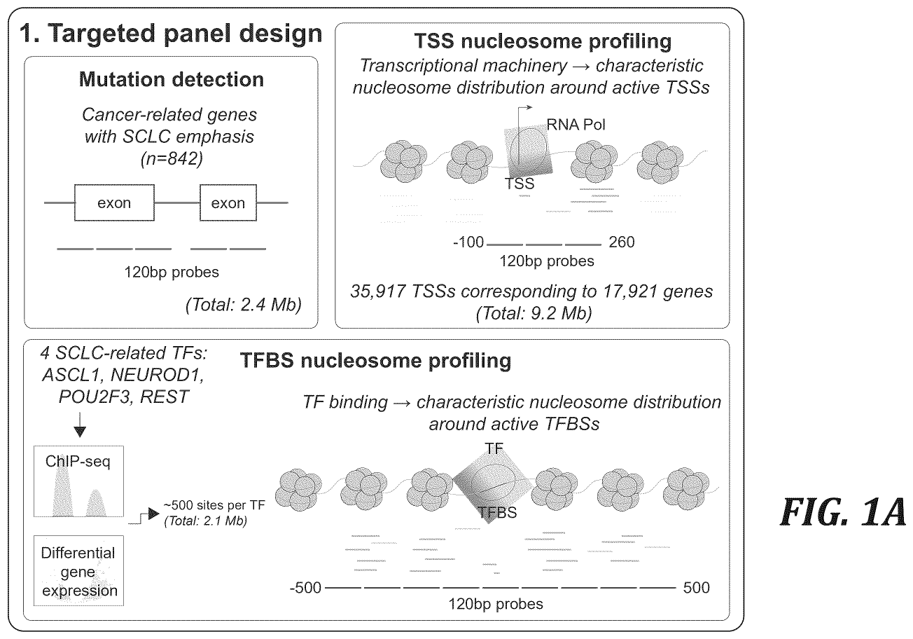

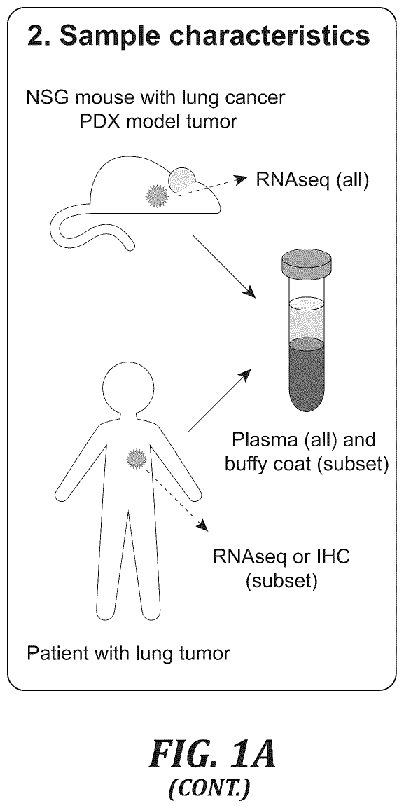

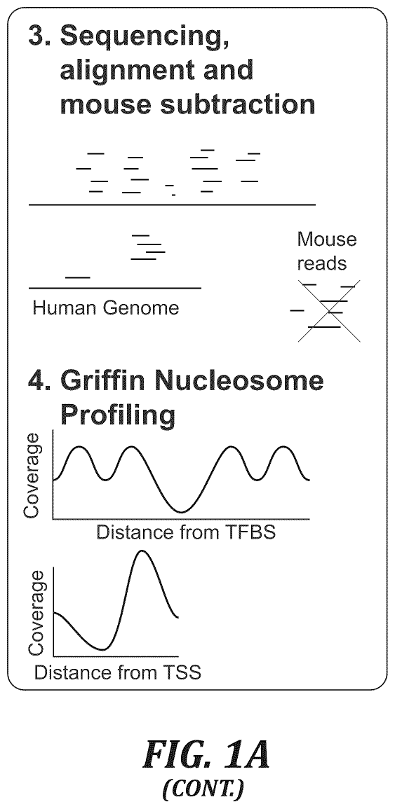

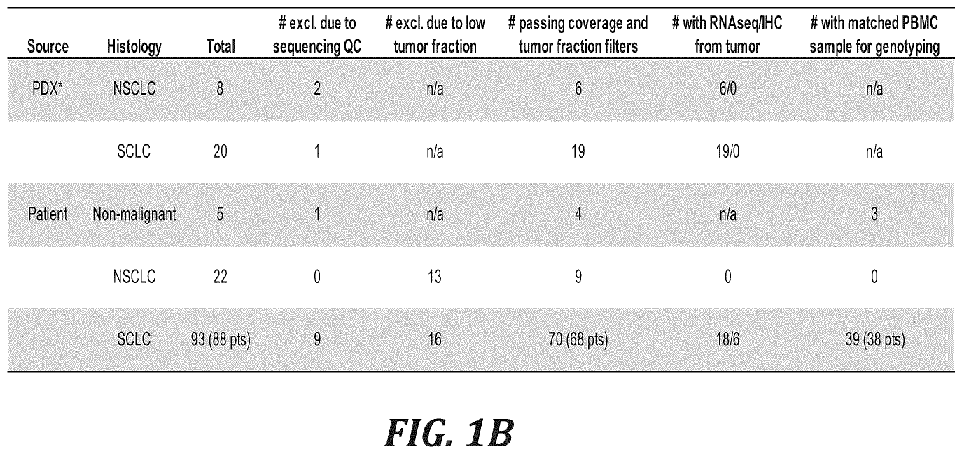

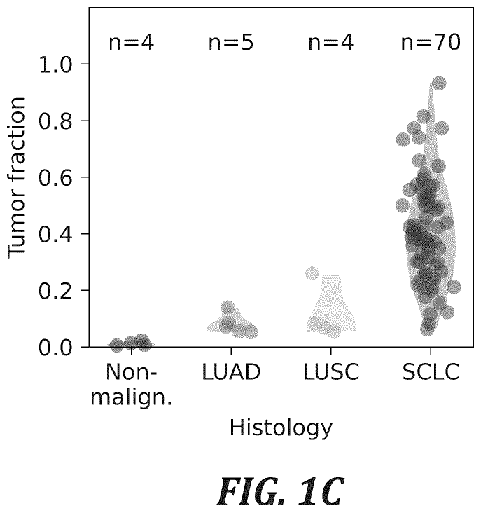

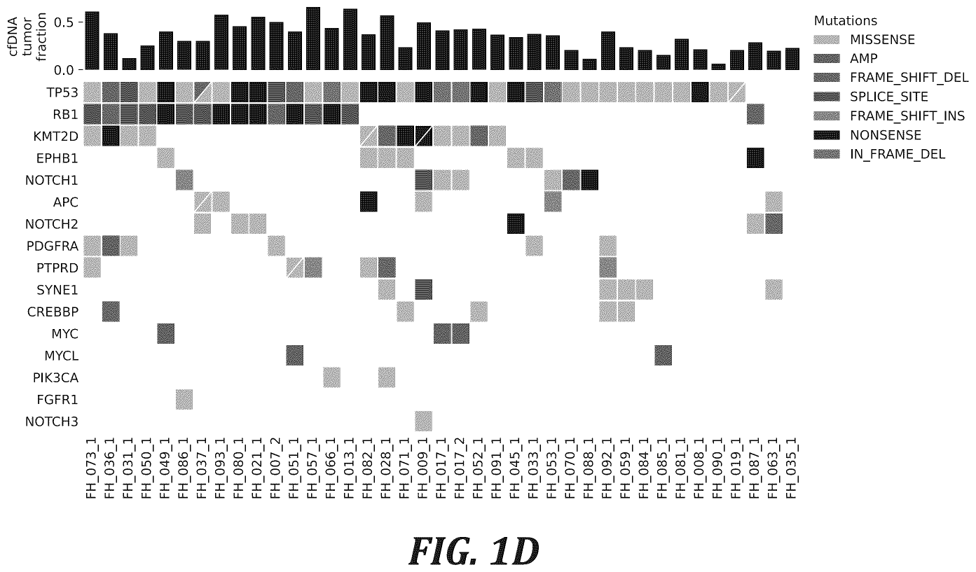

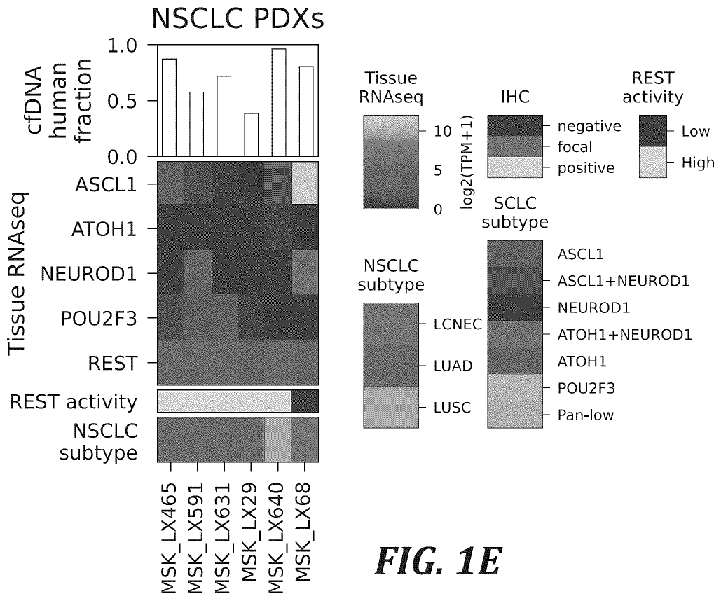

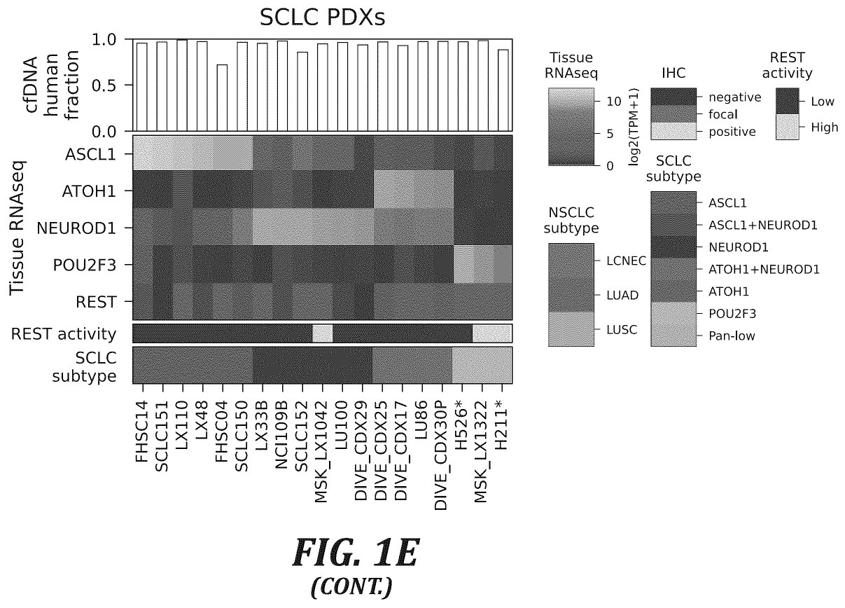

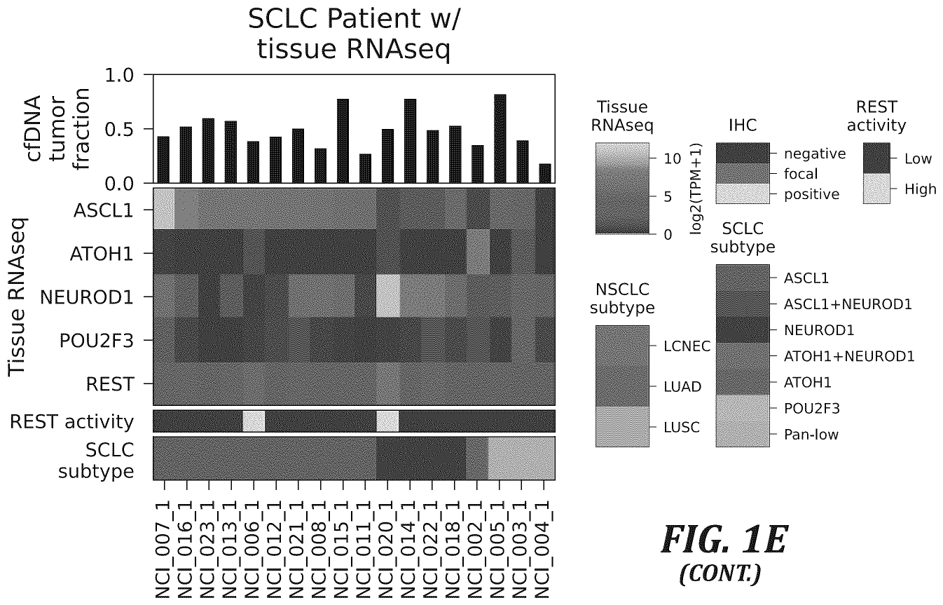

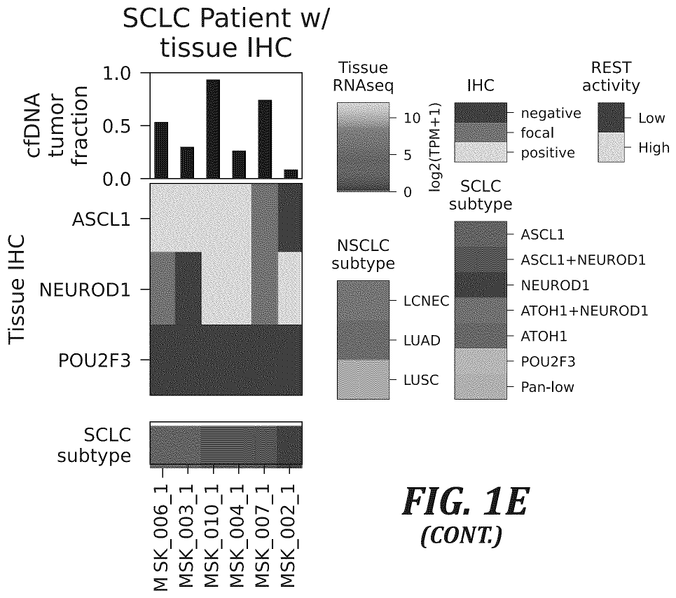

A custom targeted sequencing panel was designed to simultaneously identify somatic mutations and infer TF activity and gene expression from plasma cfDNA (FIG. 1A). Coding regions of 842 genes assembled from a published cancer gene panel were targeted (36), additional genes frequently mutated in SCLC (37,38), and genes identified in ongoing functional studies carried out in the lab of the inventors (FIG. 1A, Methods). To infer the activity of key SCLC TFs, 1 kb windows were targeted around 590 ASCL1, 414 NEUROD1, 640 POU2F3, and 781 REST TFBSs that were identified using available ChIP-seq and gene expression data (FIG. 1A, Methods). To infer expression of single genes, 360 bp windows were targeted around 35,917 TSSs corresponding to 17,921 genes (FIG. 1A, Methods). In total, the panel targeted 13.7 Mb with 120 bp probes (FIG. 1A).

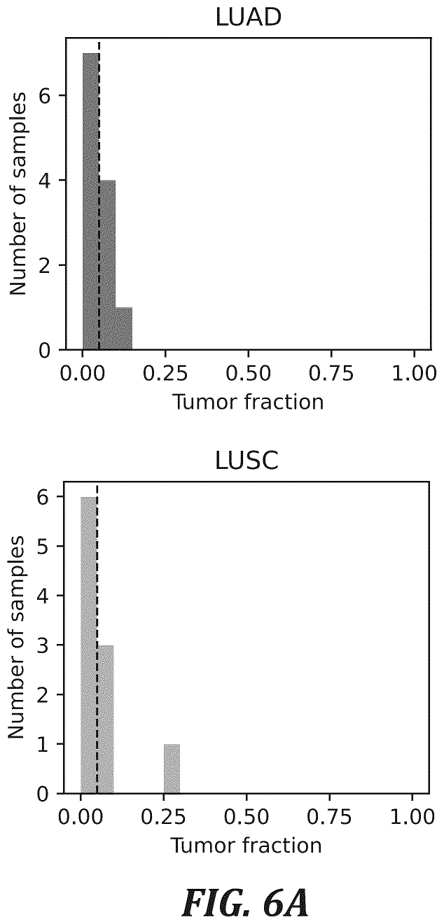

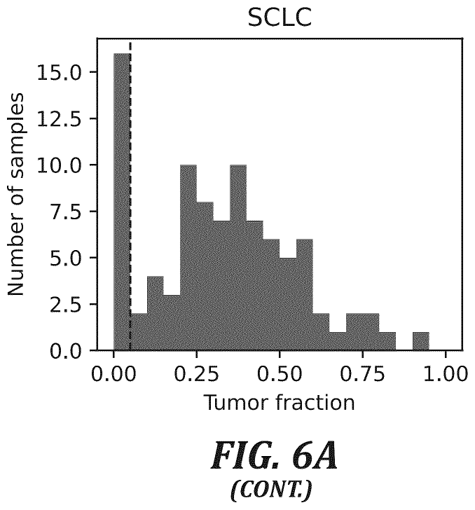

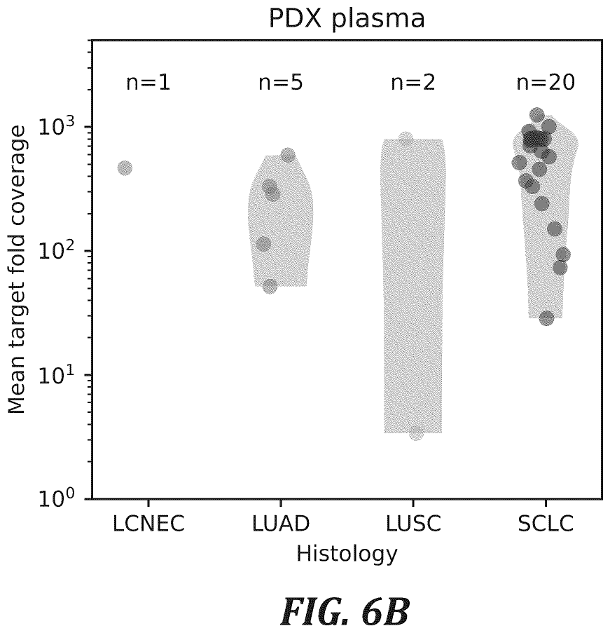

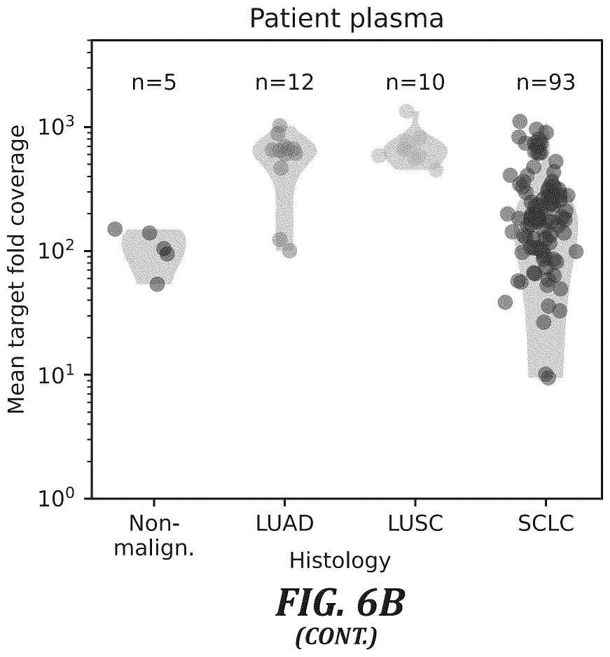

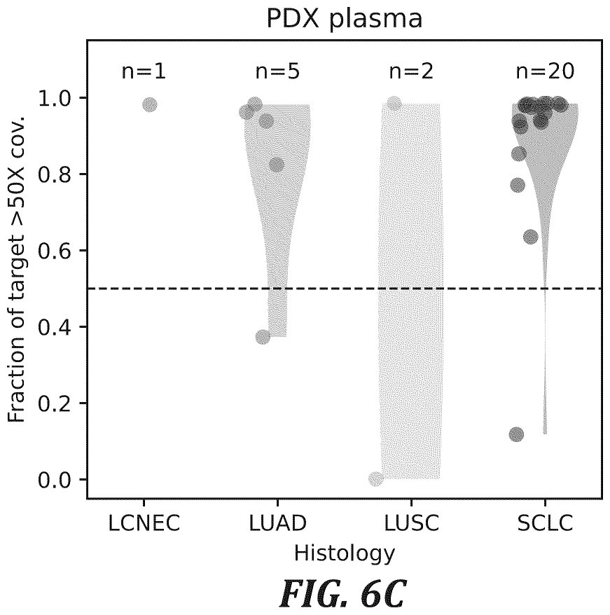

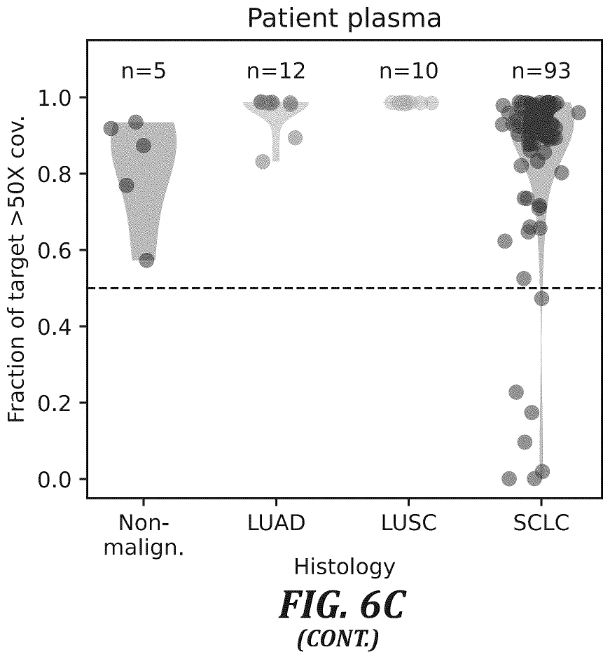

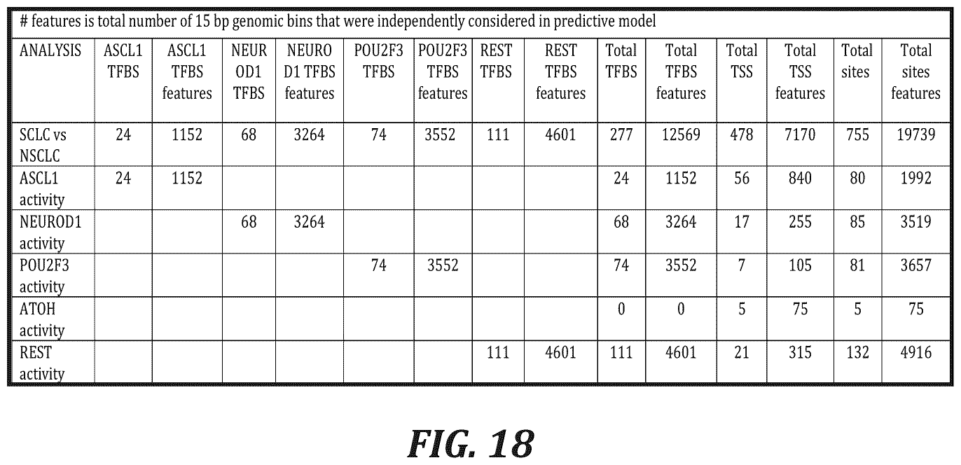

The targeted cfDNA sequencing approach was applied to plasma from mice harboring SCLC (n=20) or NSCLC (n=8) PDX models (FIG. 1B). Because of the scarcity of POU2F3-expressing PDX models, the “PDX” set used herein included one POU2F3 PDX (never passaged ex vivo) and three POU2F3-expressing SCLC cell line xenograft models. In PDX plasma samples, tumor and non-tumor cfDNA are from different species and can be separated bioinformatically to generate a pure tumor (human) cfDNA signal (34, 39). Low coverage samples were excluded, resulting in an analysis set of 19 SCLC and 6 NSCLC PDX models (FIG. 1B, FIG. 6A to 6C, Methods). Median on-target coverage of the PDX analysis set was 575× (range 73-1250×), and the median proportion of sequencing data that aligned preferentially to the human genome versus the mouse genome (termed “human fraction” and retained for subsequent analyses) was 0.95 (range 0.38-0.99) (FIG. 18).

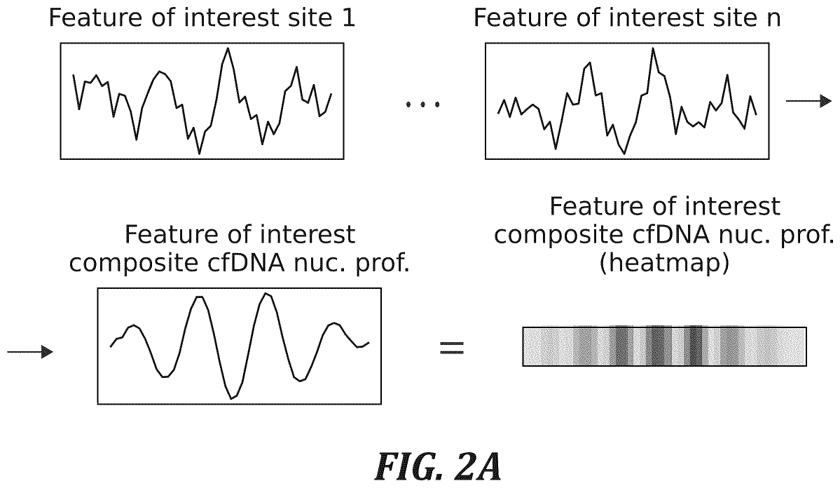

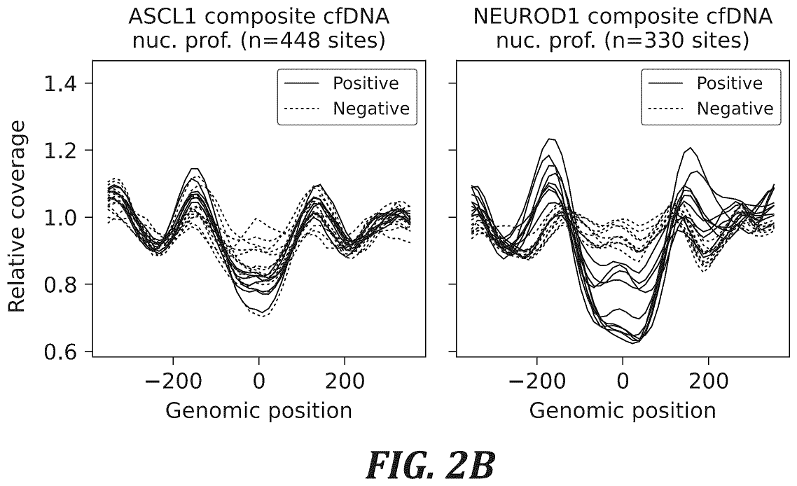

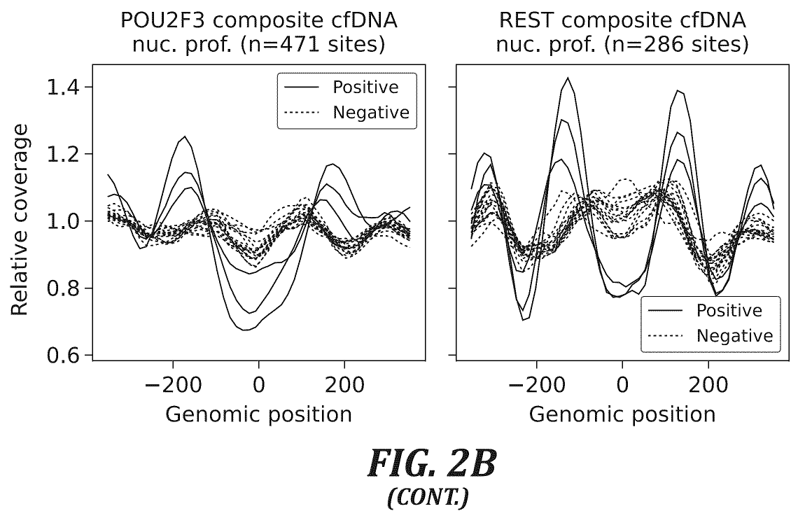

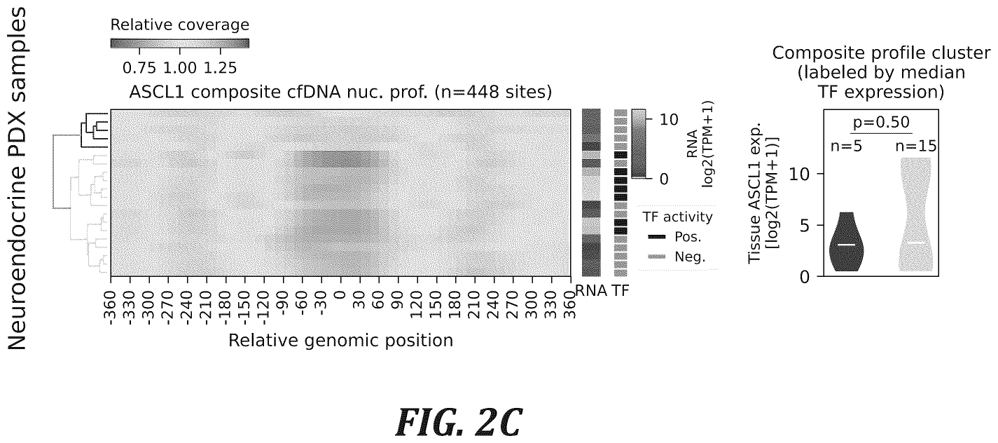

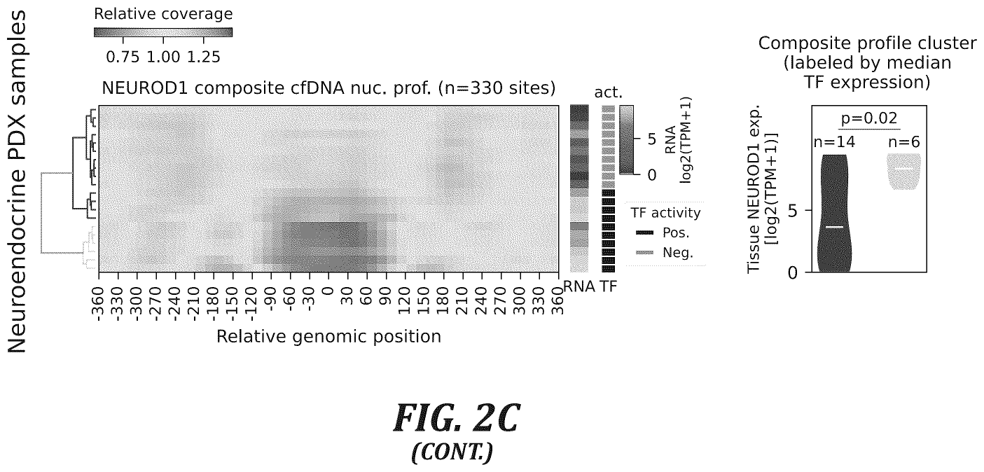

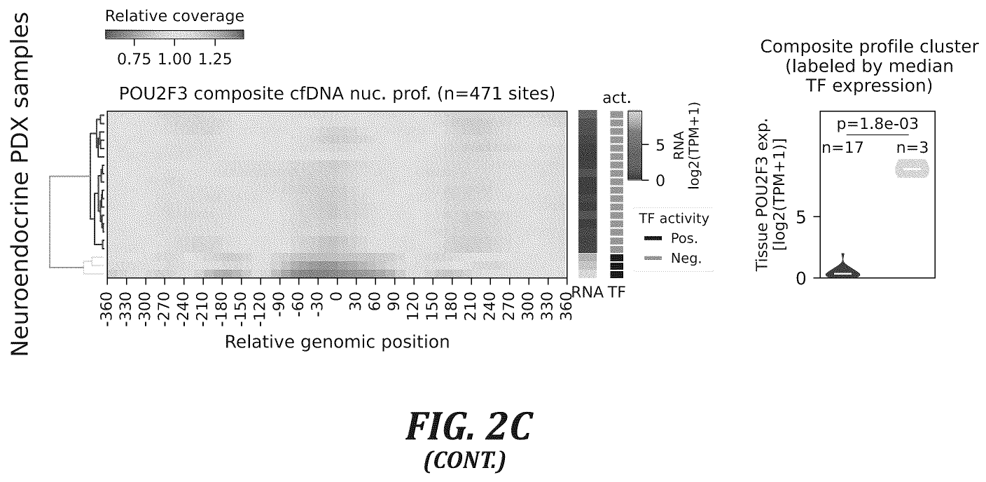

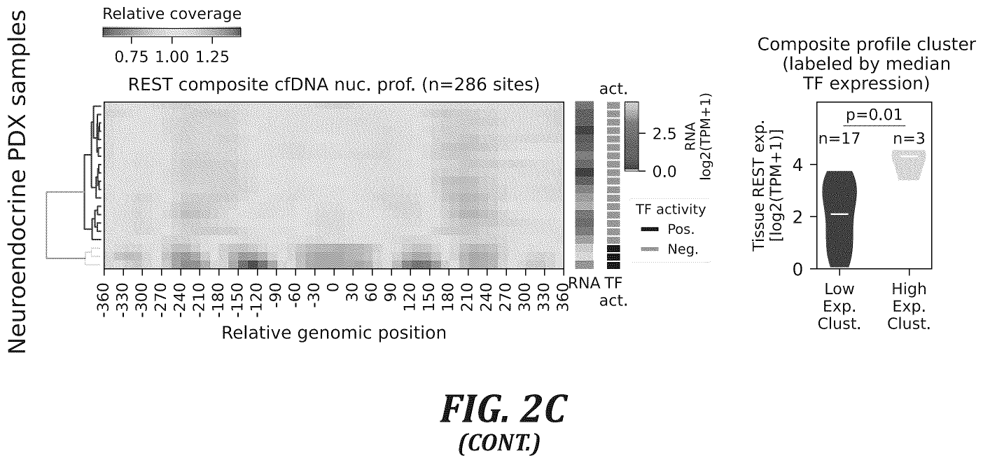

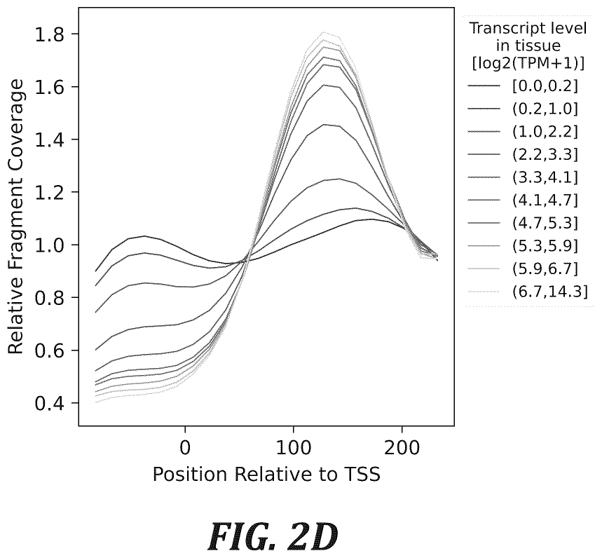

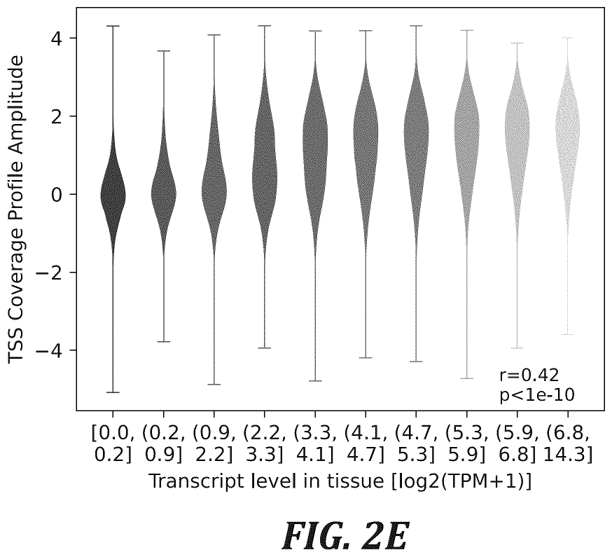

Bioinformatic subtraction of mouse sequencing reads resulted in mean 95% human reads (38%-99%) retained for analysis. The nucleosome profiles in TF-positive samples had decreased coverage indicating increased accessibility, consistent with studies from us and others, although ASCL1 signals were less pronounced (FIG. 2B). It has been previously shown that PDX plasma is the ideal resource to overcome challenges of variable ctDNA fraction and the benchmark with well-characterized matched tumor tissue for identifying sub-type specific ctDNA features; the resulting methods developed using PDX data have successfully validated using patient DNA.

Next, the targeted panel was then applied to a collection of plasma samples from patients with SCLC (n=93 samples from 88 patients), patients with NSCLC (n=22), and individuals without cancer (n=5), which are referred to as “non-malignant” (FIG. 1B). cfDNA tumor fraction was estimated analyzing separate low-pass whole genome sequencing data and excluded low coverage samples and SCLC or NSCLC samples with less than 5% tumor fraction, resulting in an analysis set of 4 non-malignant, 9 NSCLC, and 70 SCLC (n=68 patients) samples (FIG. 1B, FIG. 6, Methods). Median on target coverage of the patient sample analysis set was 212× (range 63-1108×) (FIG. 18). Median tumor fraction was 0.39 (range 0.06-0.93) in SCLC samples and 0.07 (range 0.05-0.26) in NSCLC samples (FIG. 1C), consistent with established histology-specific patterns (40).

Next, for the subset of patient samples with available matched buffy coat genomic DNA (n=39 samples from 38 patients with SCLC) (FIG. 1B), cfDNA targeted sequencing data was used to call mutations in targeted genes (FIG. 1D, Methods). Consistent with data from SCLC genomic studies (6-8) nearly universal deleterious mutations were observed in the canonical SCLC driver gene TP53 (36/39 samples, 92%) and deleterious RB1 mutations were also very frequent (16/39, 41%) (FIG. 1D). Also as expected, recurrent truncating loss of function mutations were observed in SCLC tumor suppressors such as KMT2D, NOTCH1/2 and CREBBP, and recurrent amplifications in MYC and MYCL (FIG. 1D). Together, these results establish that the custom sequencing panel generates high coverage at sites of interest and identifies mutations in key SCLC related genes.

Finally, to serve as a benchmark for analyses of TF activity and gene expression from cfDNA, matched tumor tissue was analyzed using RNAseq or IHC for all PDX samples and for patient samples where available (FIG. 1E, Methods). Using these data, SCLC samples were classified into subtypes based on the expression of ASCL1, ATOH1, NEUROD1, and POU2F3 (Methods), revealing a diverse spectrum of subtypes: ASCL1 (n=6 PDX, 12 patient), ASCL1+NEUROD1 (n=0, 3), NEUROD1 (n=6, 5), ATOH1+NEUROD1 (n=4, 0), ATOH1 (n=0, 1), POU2F3 (n=3, 0), and pan-low (n=0, 3) (FIG. 1E). REST is most commonly active in POU2F3-positive SCLC but can also be active in other SCLC subtypes and is highly active in LUAD and LUSC NSCLC (41); therefore, a REST activity label was separately applied to all samples (Methods), revealing 3 REST-active SCLC PDX models and 2 REST-active SCLC patient samples (FIG. 1E).

Tumor Cell TF Activity and Gene Expression Signatures were Apparent in Targeted cfDNA Nucleosome Profiling Data

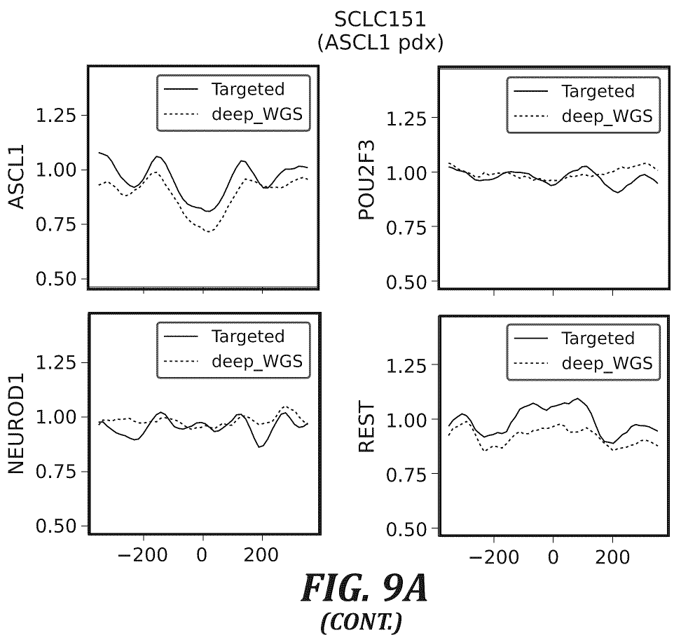

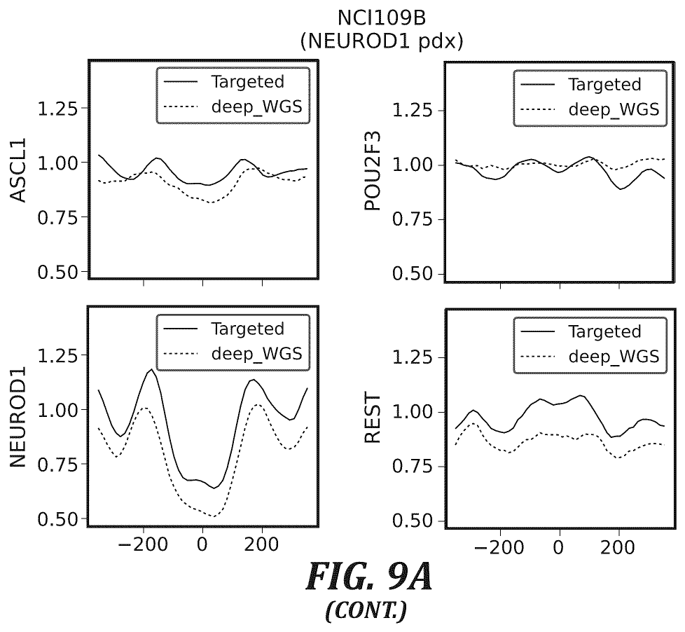

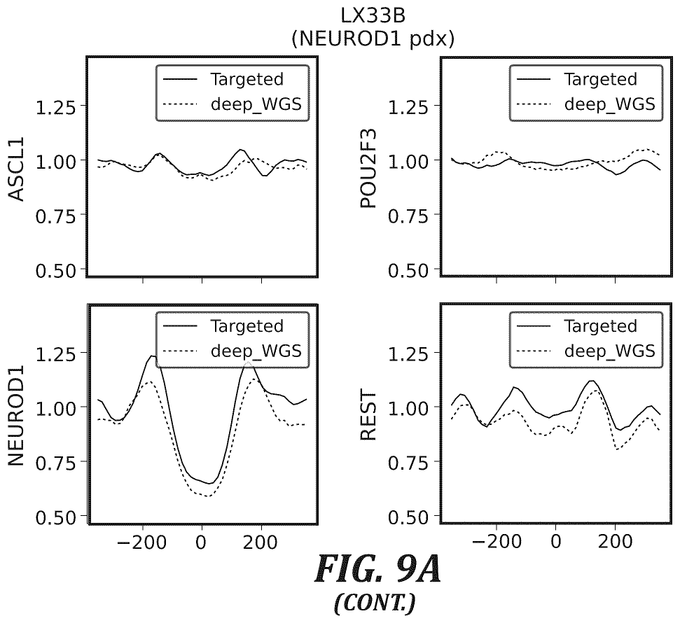

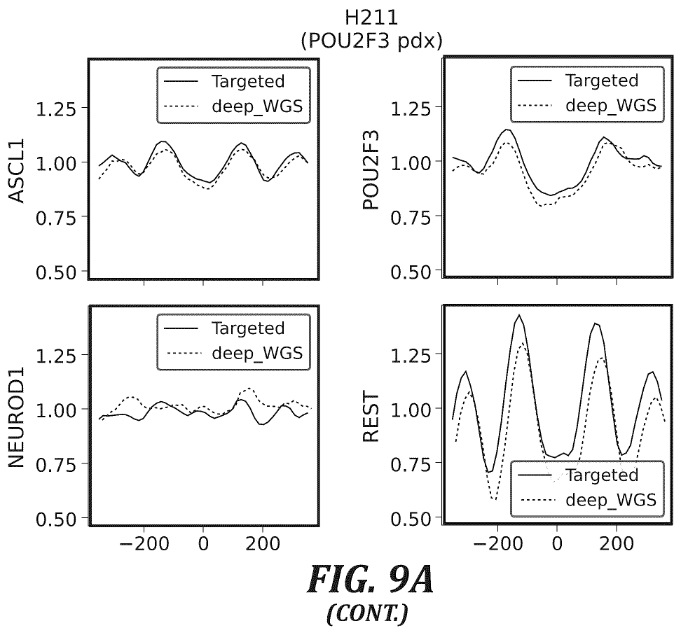

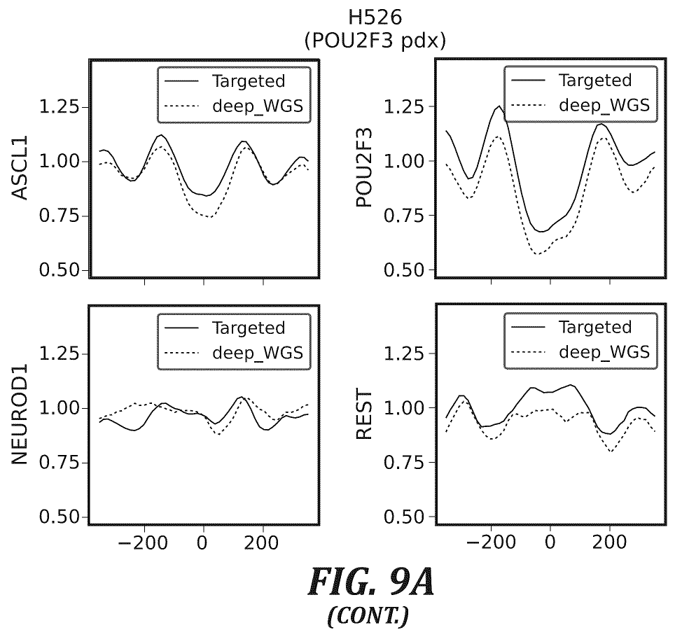

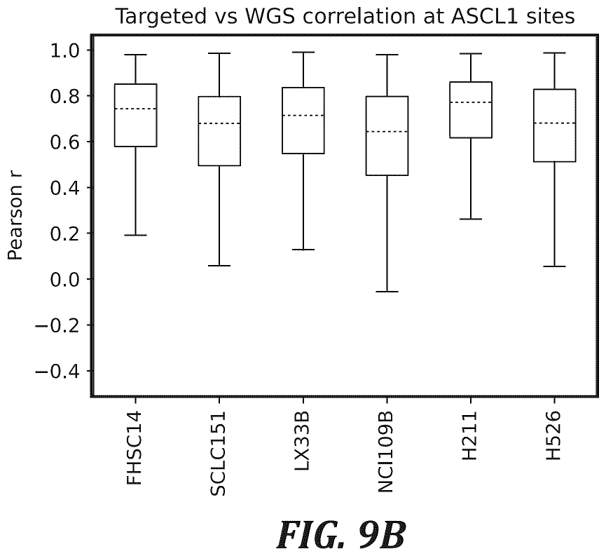

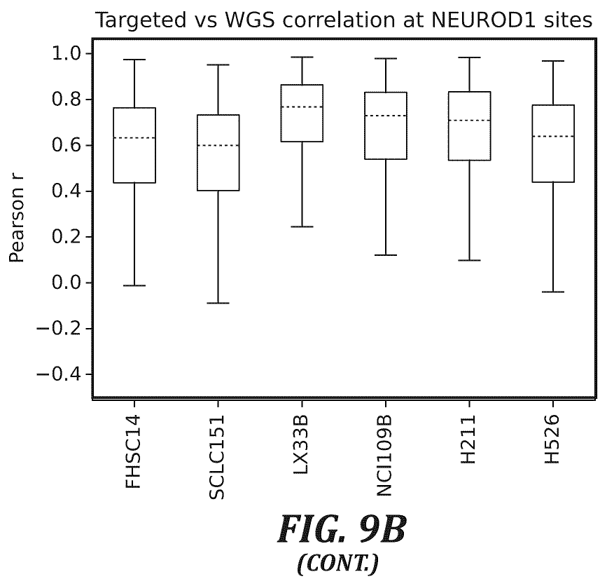

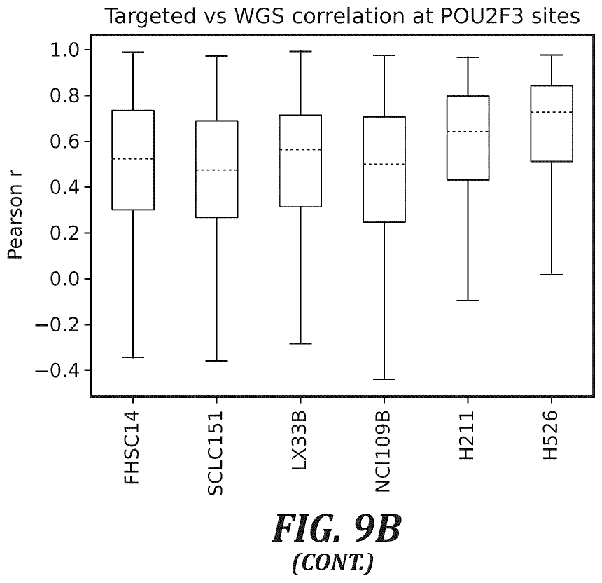

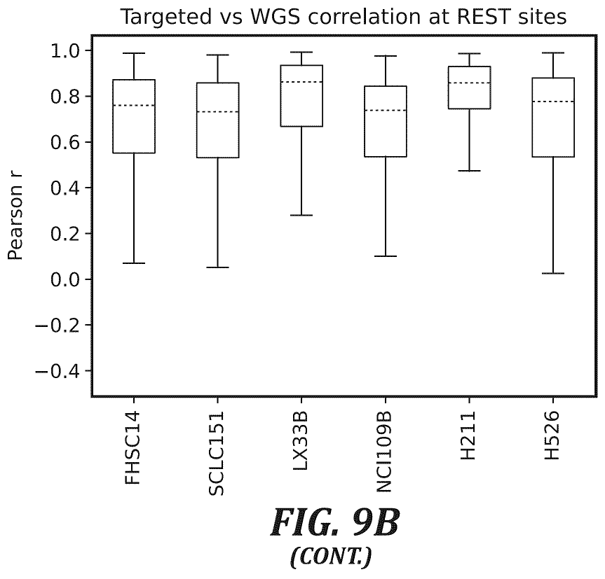

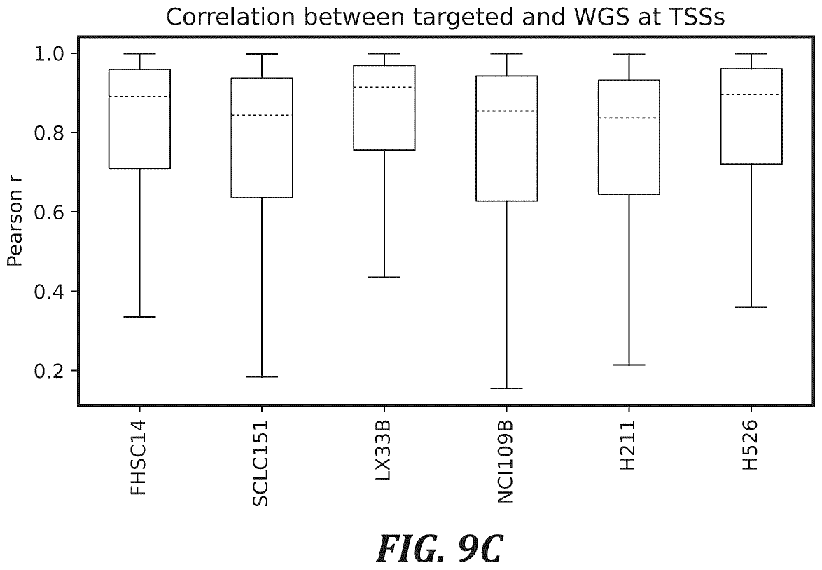

It was then asked whether targeted sequencing of TFBSs in cfDNA reveals evidence of tumor cell TF activity in neuroendocrine (NE) PDX samples (n=19 SCLC and 1 LCNEC). To generate cfDNA nucleosome profiles at TFBSs, the recently described Griffin cfDNA nucleosome profiling computational framework (33) was applied for use with targeted sequencing data, which resulted in a set of 1,535 TFBSs that passed quality control filters (FIG. 8, Methods). A single “composite” cfDNA nucleosome profile was then generated for each TF in each sample by averaging the Griffin signal for all TF-linked TFBSs in the targeted panel (FIG. 2A). To validate the fidelity of targeted cfDNA nucleosome profiling, composite and individual site profiles were compared to deep cfDNA whole genome sequencing (WGS) from 6 PDX samples (median 93×), which revealed concordance in coverage patterns for both composite and individual TFBS nucleosome profiles (median Pearson's r range 0.63-0.74) (FIGS. 9A-9C).

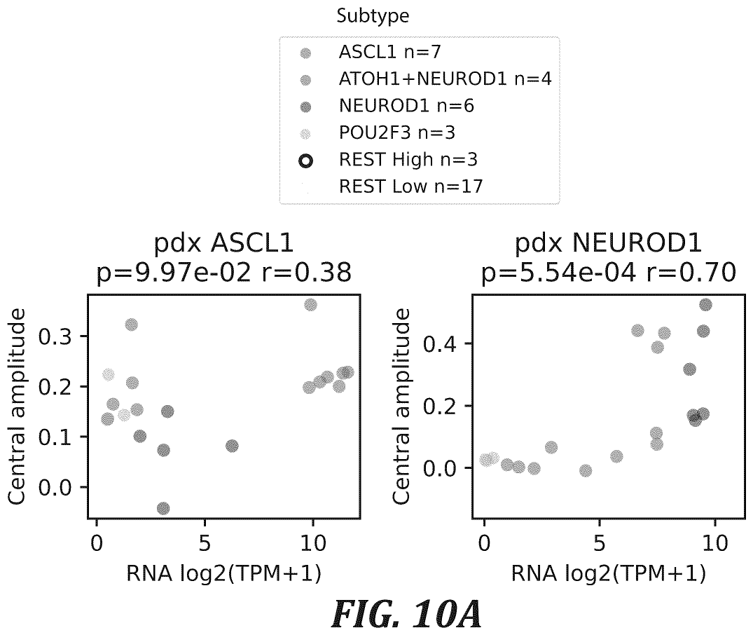

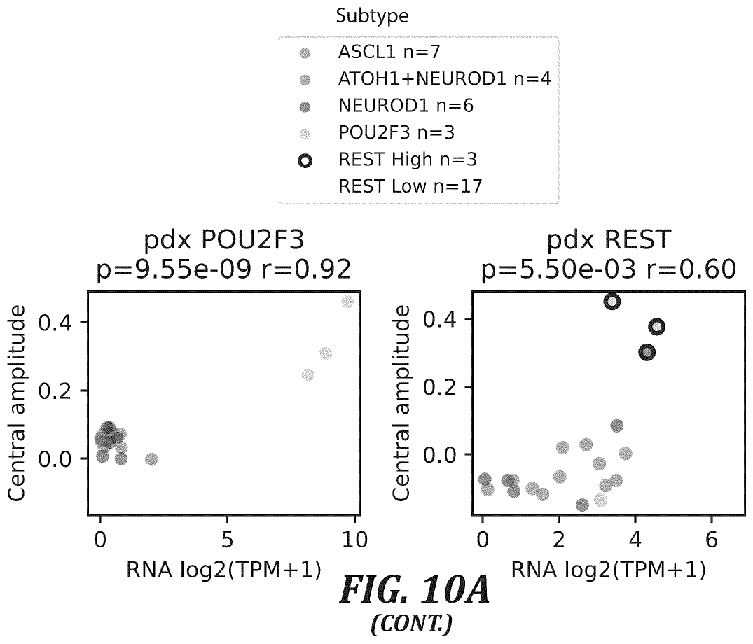

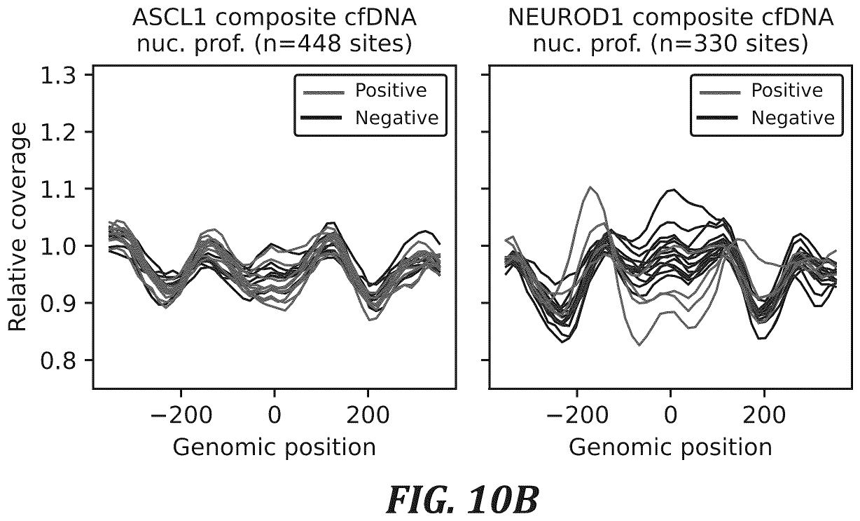

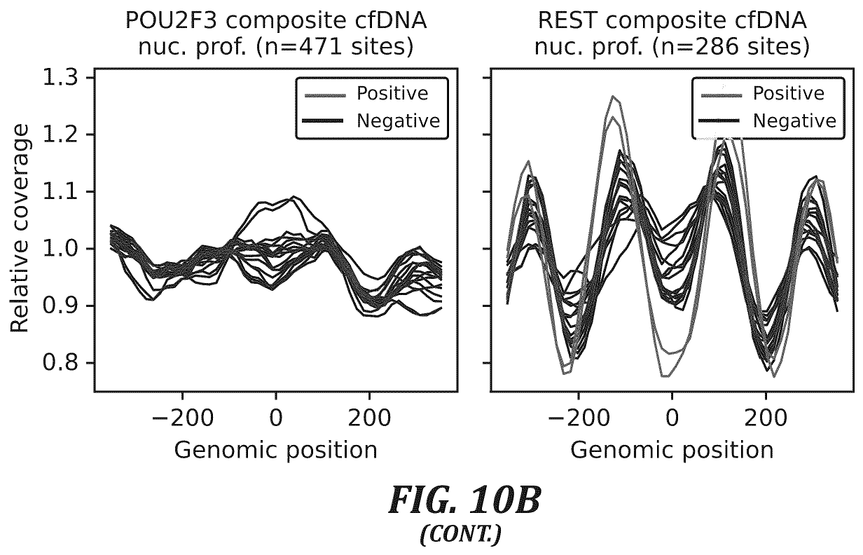

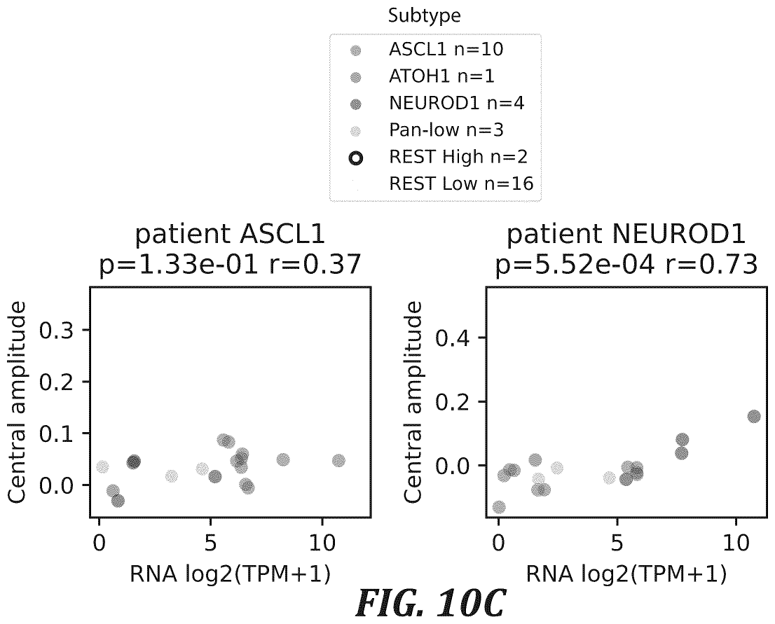

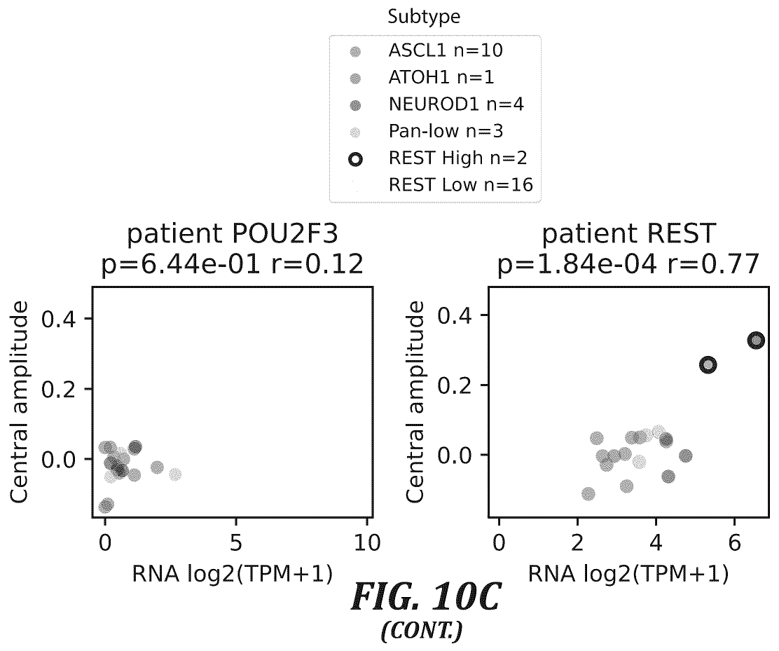

In targeted cfDNA nucleosome profiling of all 20 NE PDXs, composite nucleosome profiles in TF-positive samples appeared substantially more variable than in TF negative samples with decreased coverage at the TFBS relative to the flanking regions, consistent with previous studies (FIG. 2B) (29, 42). The magnitude of the coverage difference at the TFBS relative to the flanking region, which were termed “central amplitude,” was significantly correlated with NE PDX tissue TF transcript level for NEUROD1 (Pearson's r=0.70, p=5.5e-4), POU2F3 (r=0.92, p=1e-8), and REST (r=0.60, p=5.5e-3), but not ASCL1 (r=0.38, p=0.10) (FIG. 10A). Similarly, unsupervised hierarchical clustering of the composite profiles discriminated samples by TF activity for NEUROD1, POU2F3, and REST (two-sided Mann-Whitney U test p value range 0.002-0.02) but not ASCL1 (FIG. 2C).

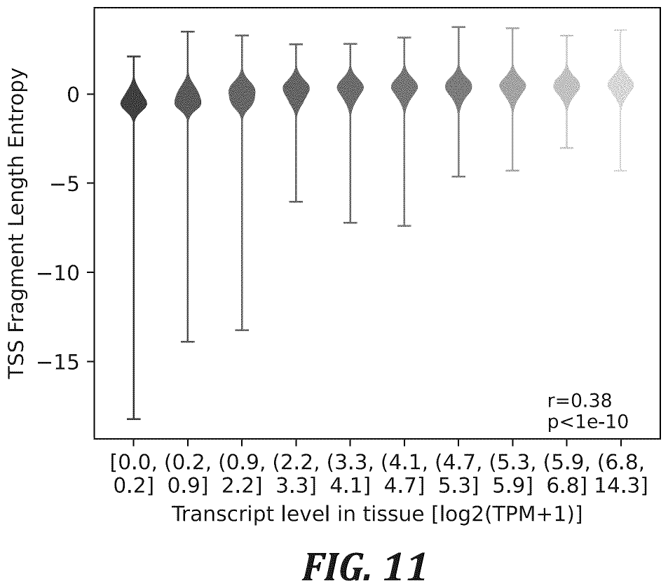

It was then asked whether targeted cfDNA nucleosome profiles at TSSs reveal evidence of tumor cell gene expression, focusing on a set of 13,240 TSSs that were selected for transcript abundance, available gene expression data, and adequate coverage in cfDNA libraries (Methods). Despite targeting a relatively narrow 360 bp window, targeted TSS cfDNA nucleosome profiles were strongly correlated with those in WGS data (median Pearson's r range 0.84-0.91) (FIGS. 9A-9C). In targeted cfDNA sequencing of the 20 NE PDXs, composite TSS cfDNA nucleosome profiles were progressively more non uniform with increasing gene expression decile, consistent with previous reports (30, 35, 43, 44) (FIG. 2D). Next, it was examined whether the high depth of coverage at individual TSSs in the acquired data would enable single gene expression inference. The magnitude of the coverage difference between the +120 and −45 positions at an individual TSS, which was termed TSS “amplitude,” was positively correlated with gene transcript level (Pearson's r=0.42, p<1e-10) (FIG. 2E), although there was considerable overlap across gene expression deciles. Fragment length entropy, which has been proposed to predict gene expression from cfDNA (43), exhibited a similar pattern (Pearson's r=0.38, p<1e-10) (FIG. 11). Altogether, these results establish that highly multiplexed targeted cfDNA nucleosome profiling at TFBSs and TSSs faithfully reveals signals of tissue TF activity and gene expression in NE PDXs.

### Analysis of Selected Highly Informative Sites Preserved Strong Signals of TF Activity and Gene Expression

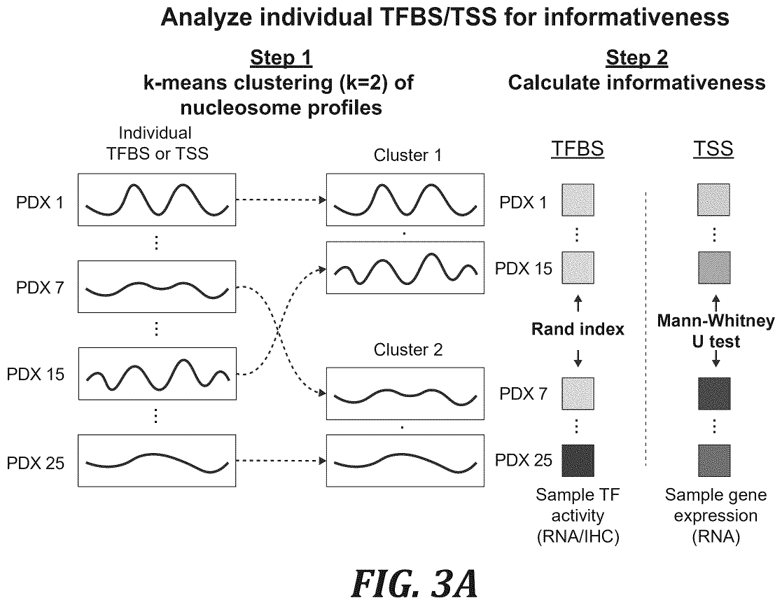

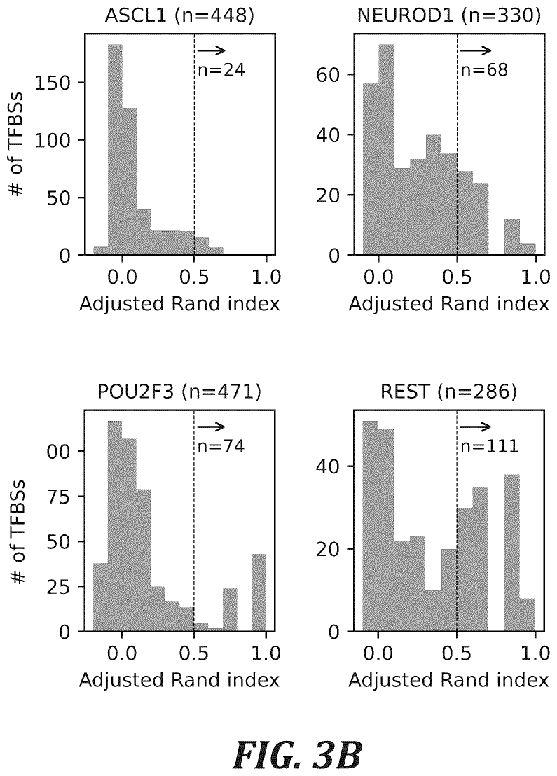

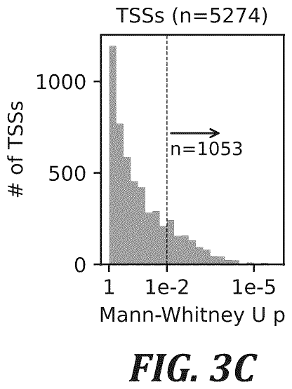

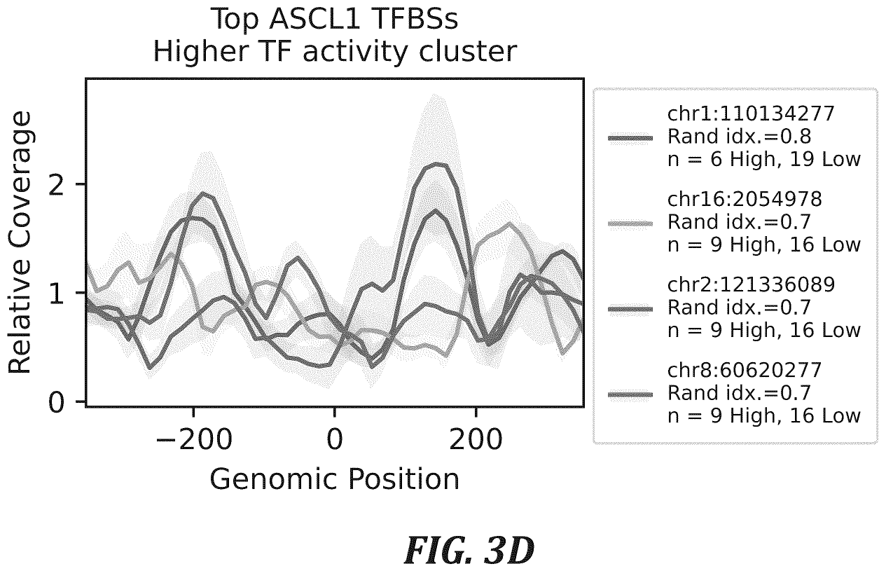

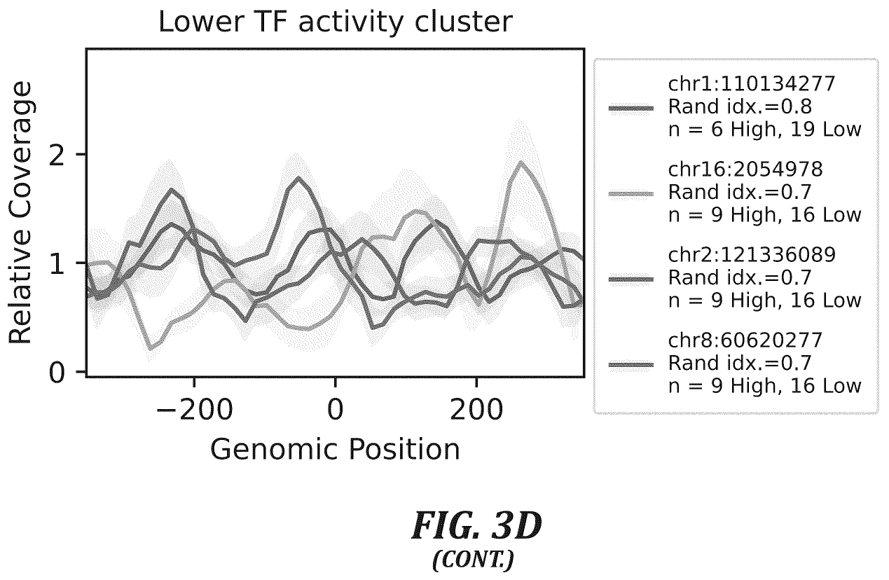

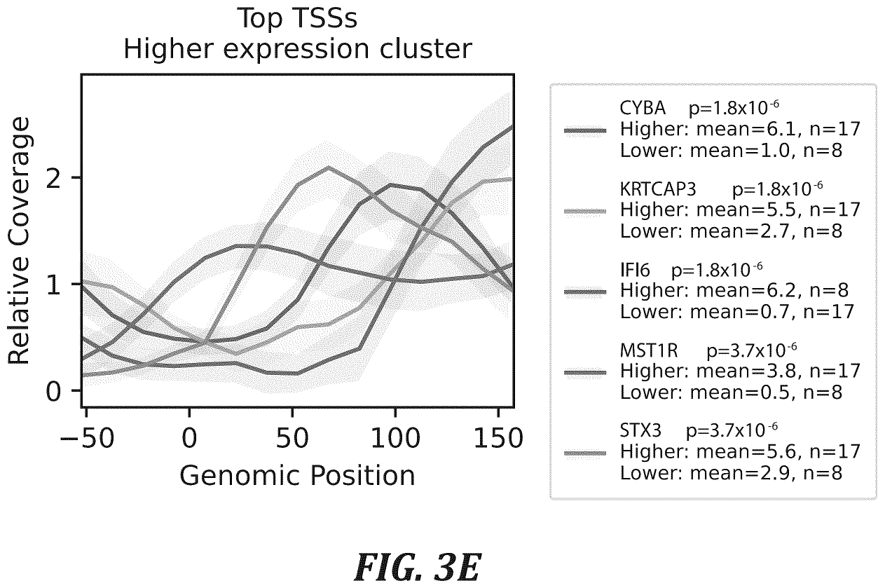

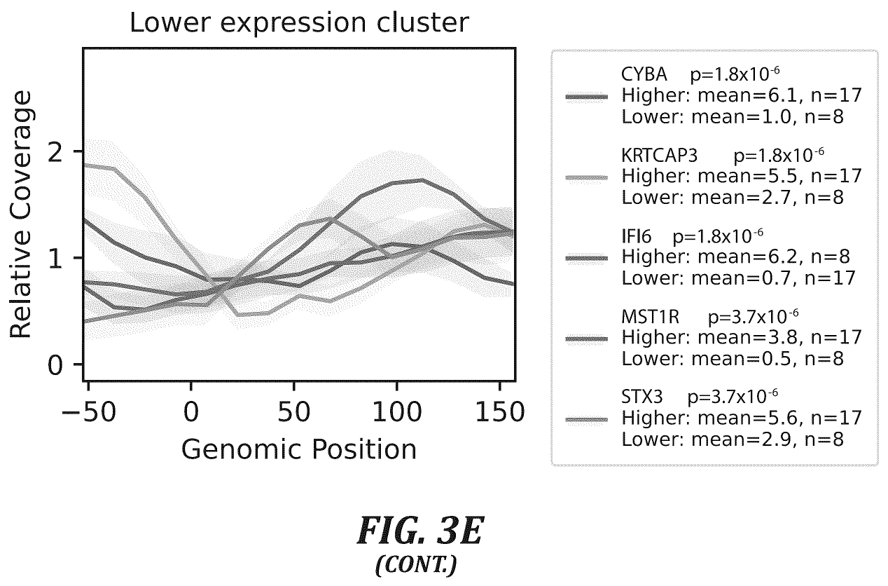

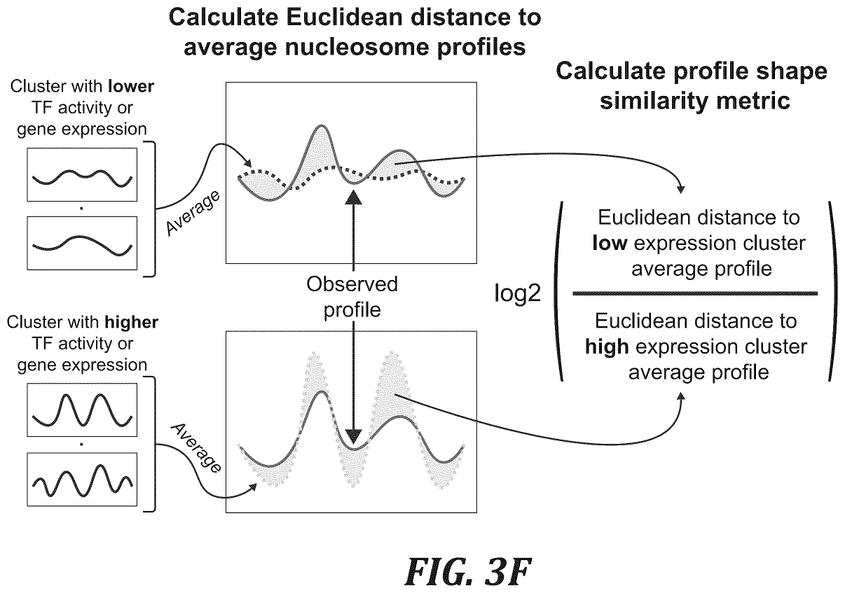

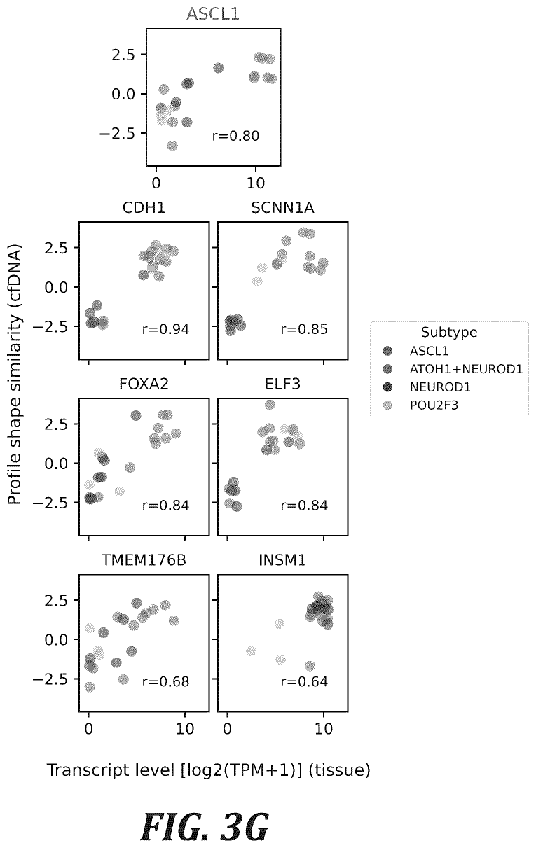

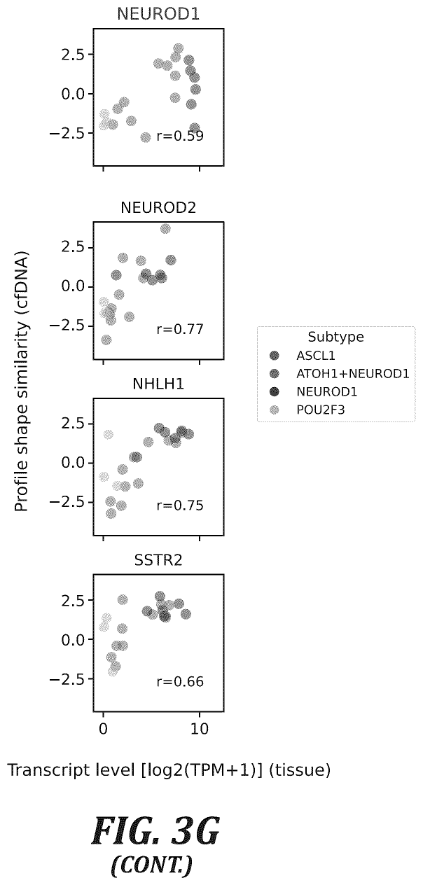

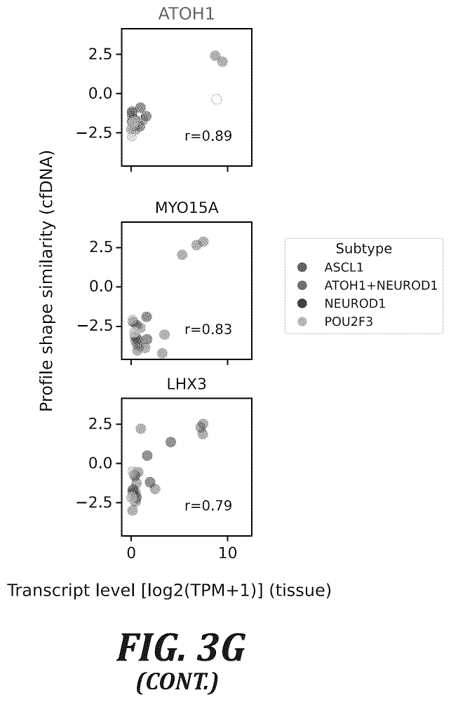

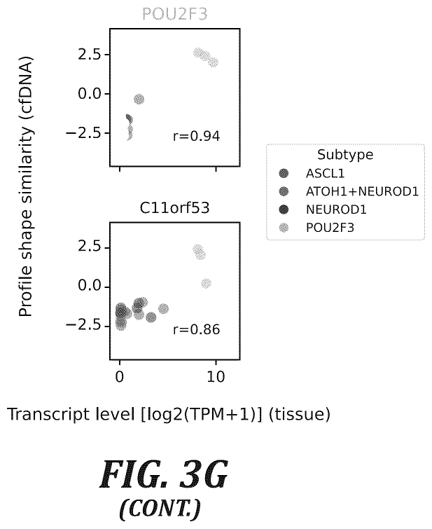

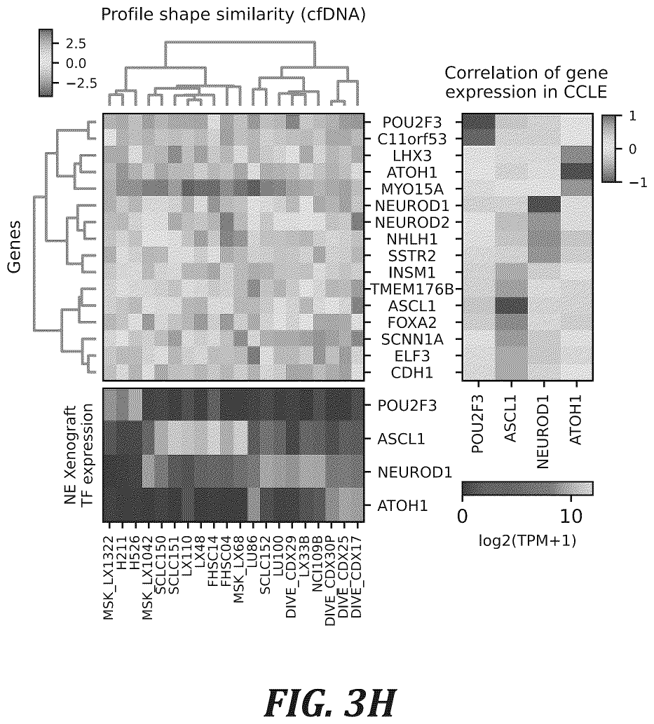

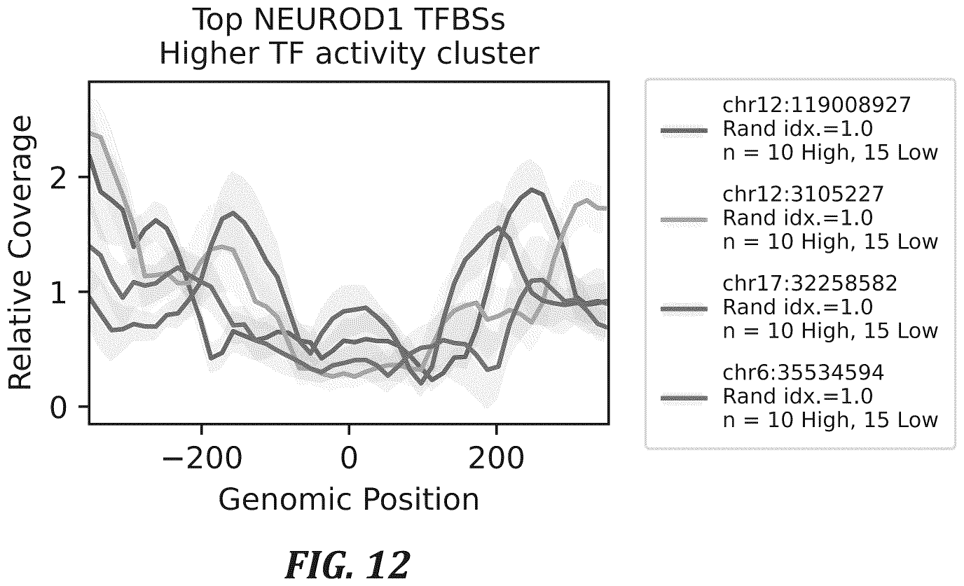

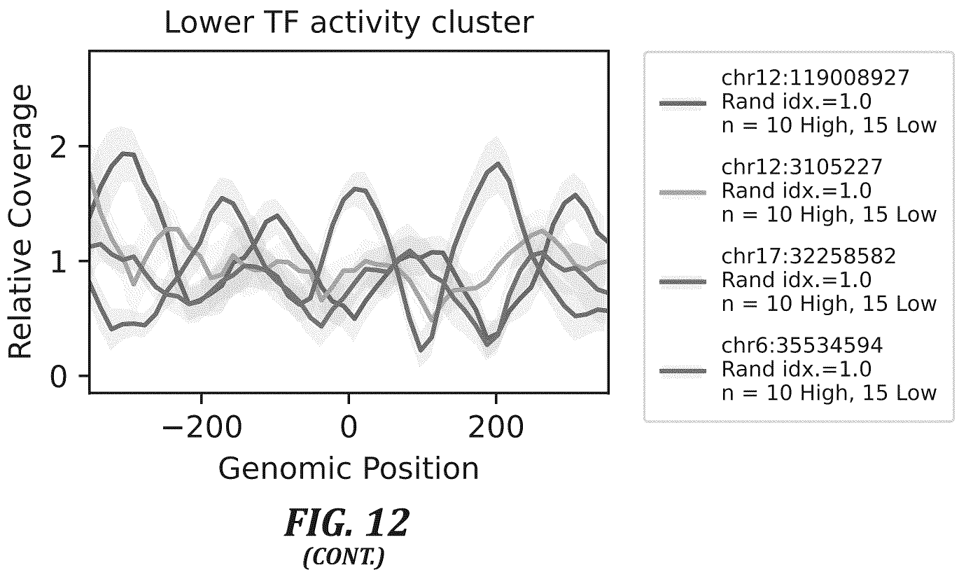

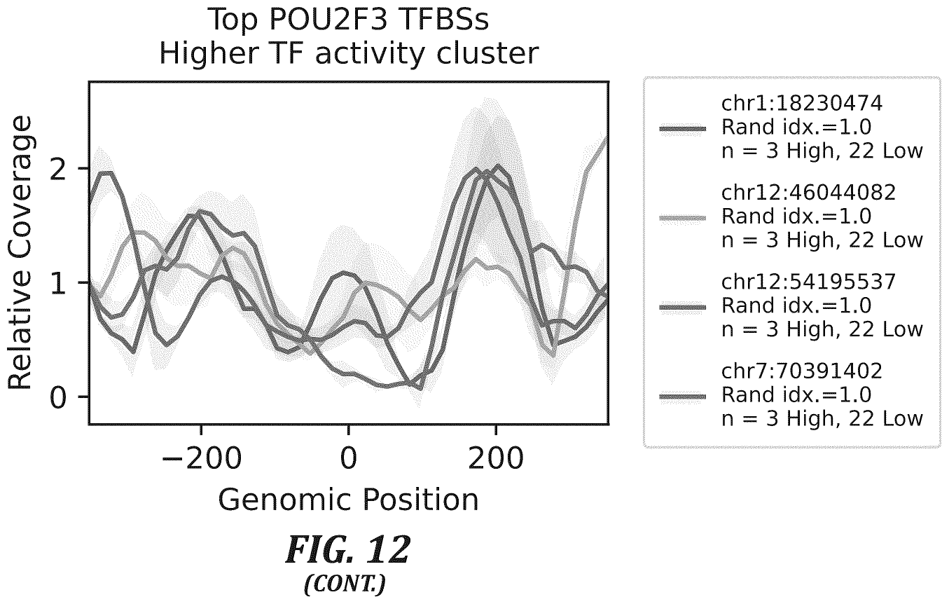

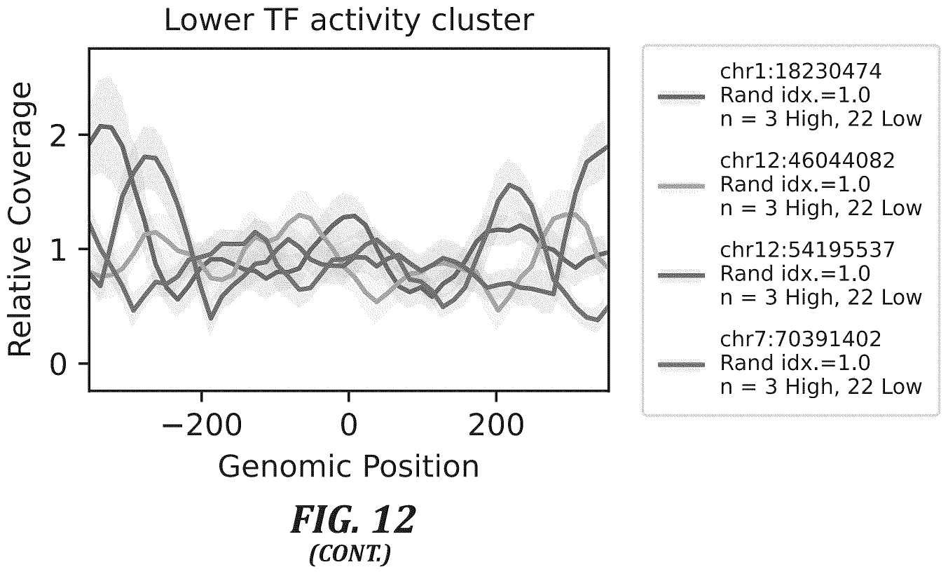

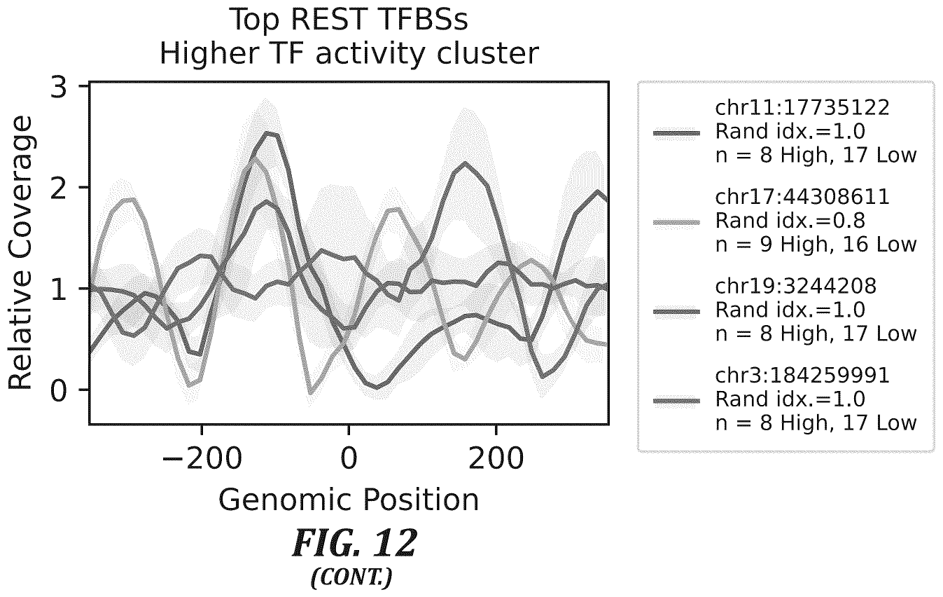

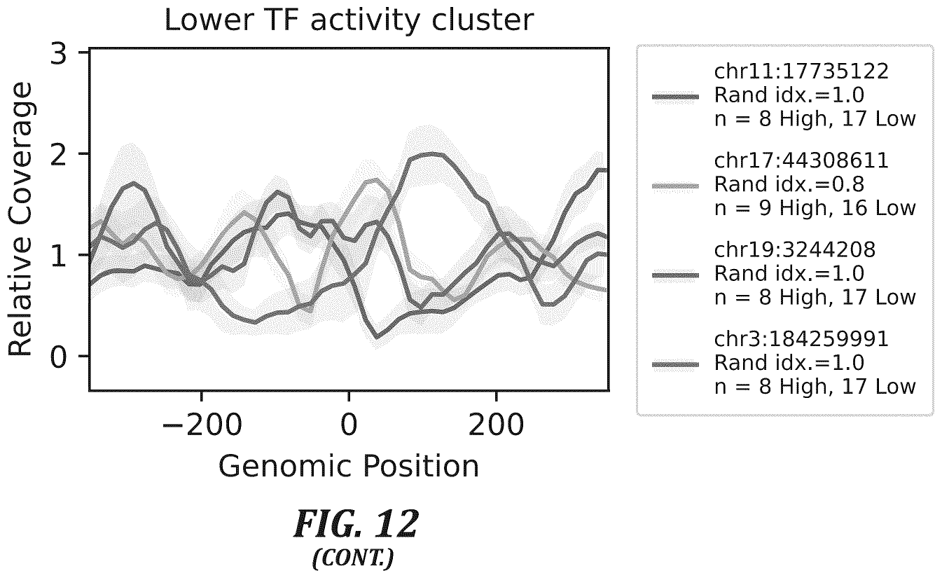

Chromatin accessibility patterns can exhibit substantial heterogeneity across genomic loci of interest (45). Therefore, it was explored whether accurate assessment of cfDNA nucleosome profiles at individual sites would support more sensitive inference of TF activity and gene expression (45). In contrast to low coverage WGS, high coverage targeted sequencing facilitates detailed examination of individual cfDNA nucleosome profiling sites. Therefore, it was sought to refine the individual sites used to infer TF activity (TFBSs) or gene expression (TSSs). Separate unsupervised k-means clustering (k=2) of the cfDNA nucleosome profiles were performed at each of the 1535 TFBSs across the PDX samples (n=25) and quantified individual TFBS “informativeness” using the adjusted Rand index, which compares sample cluster assignments with tissue TF activity labels (FIG. 3A, Methods). It was found that only 18% of TFBSs (277 of 1535) exhibited an adjusted Rand index of at least 0.5, signifying good agreement between cfDNA nucleosome profile cluster assignment and TF activity label (FIG. 3B). Of 5,274 TSSs associated with variably expressed genes in the PDX samples, using an analogous strategy (FIG. 3A, Methods), it was found that 20% had a strong association (two sided Mann-Whitney U test p<0.01) between cluster assignment and expression of the associated gene (FIG. 3C). Next, it was asked whether cfDNA nucleosome profiles at highly informative sites exhibited similar or divergent profile shapes, as the composite analysis strategy used above assumes similar profile shapes between sites. Visually divergent average nucleosome profiles of TF active samples were observed at the 4 most informative TFBSs ranked by adjusted Rand index (FIG. 3D, FIG. 12) and of samples highly expressing a given gene at the 5 most informative TSSs ranked by Mann Whitney U test p value (FIG. 3E). These results indicate that targeted sequencing can reveal considerable heterogeneity between sites and suggest that tissue TF activity and gene expression may be detectable via individual analysis of carefully selected sites.

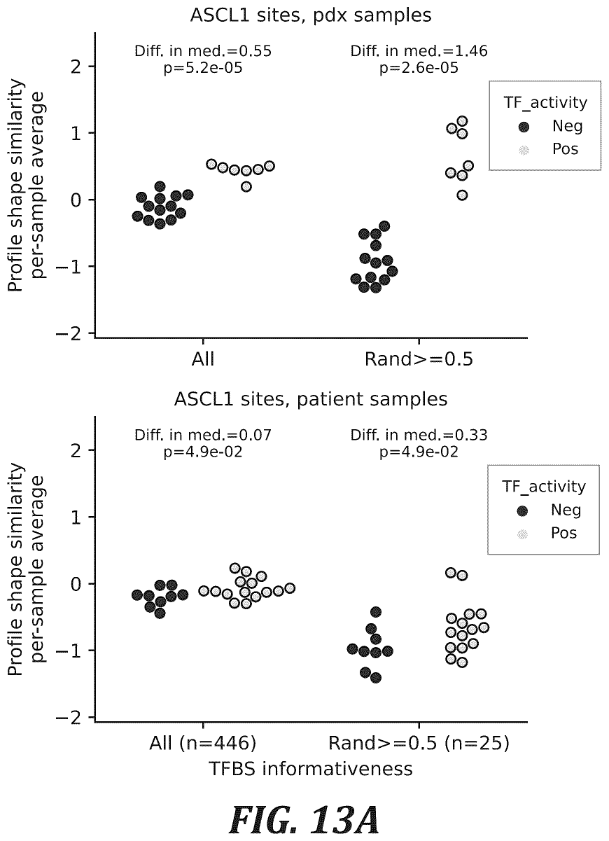

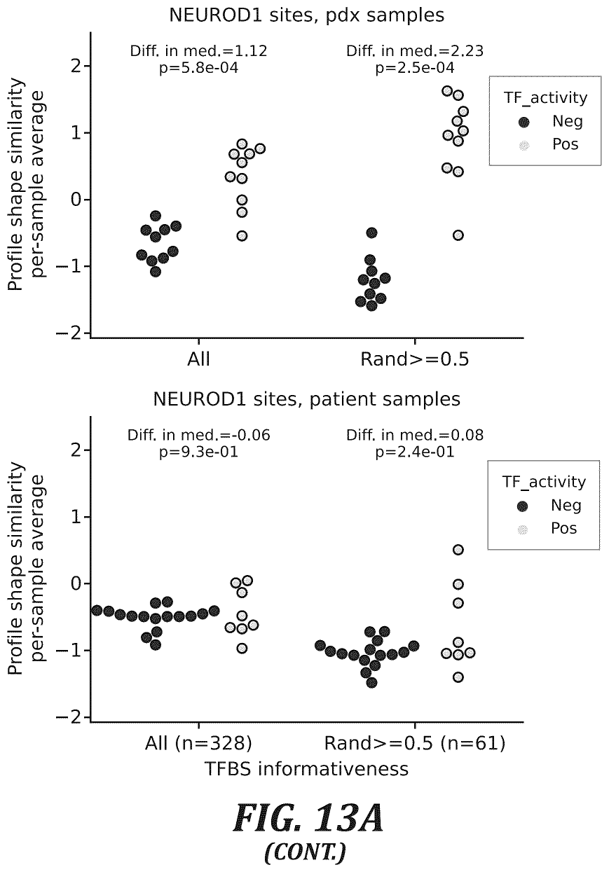

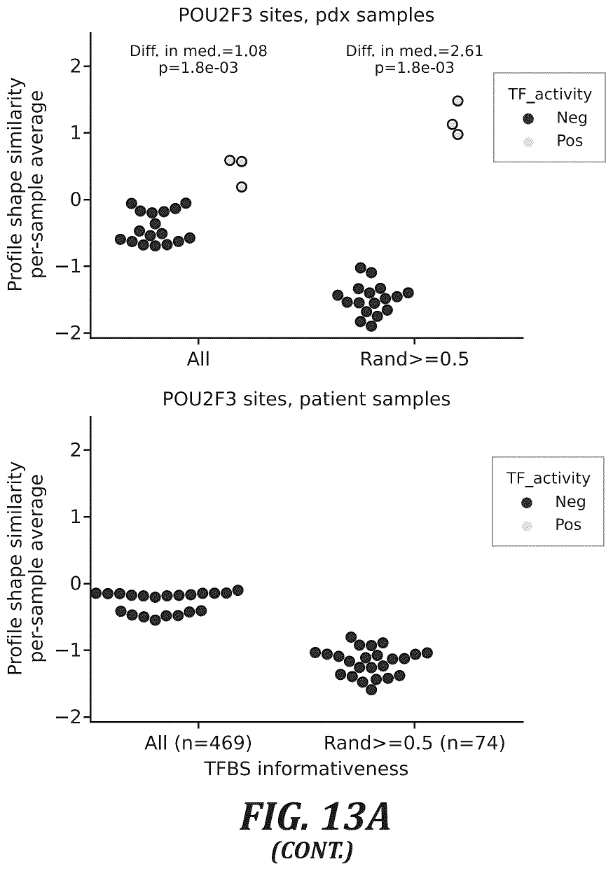

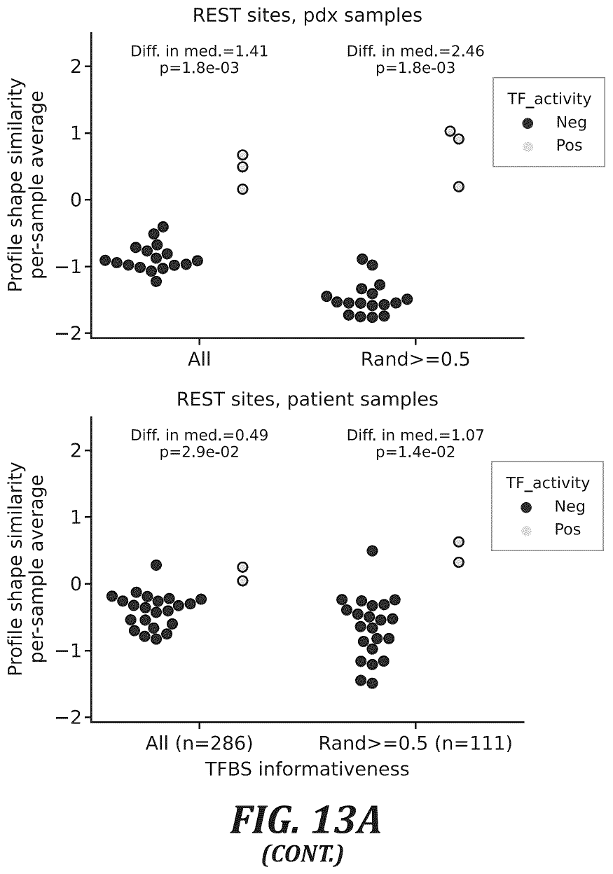

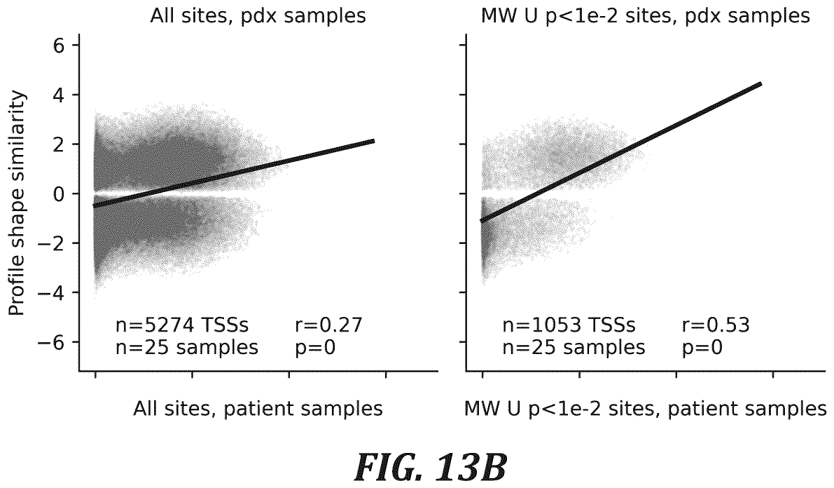

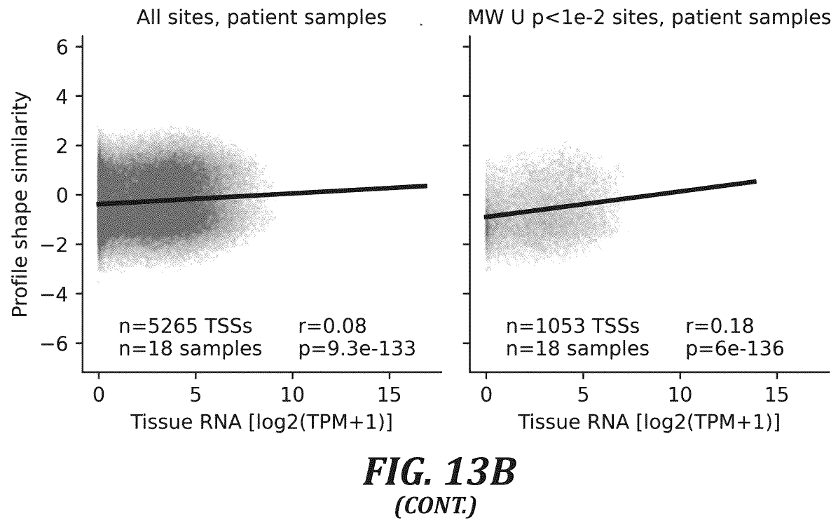

It was then sought to confirm that the most informative sites identified in PDX cfDNA samples were also more informative in cfDNA from SCLC patients. Considering the observed heterogeneity between sites, we developed a method to interpret cfDNA nucleosome profiles in each sample without a prespecified expectation of characteristic nucleosome profile shapes associated with TF activity or gene expression states. At an individual TFBS or TSS, a “profile shape similarity” metric was computed based on Euclidean distance to quantify the relative similarity of an observed cfDNA nucleosome profile in a given sample to the average profile observed across samples with higher or lower TF activity (for TFBSs) and higher or lower gene expression (for TSSs) (FIG. 3F, Methods). Profile shape similarity scores were more strongly related to TF activity or gene expression at highly informative sites (TFBSs with adjusted Rand index≥0.5 or TSSs with Mann-Whitney U test p<0.01) compared to all sites in SCLC patient cfDNA samples (FIGS. 13A and 13B), supporting the generalizability of informative site selection using targeted cfDNA data from PDX samples.

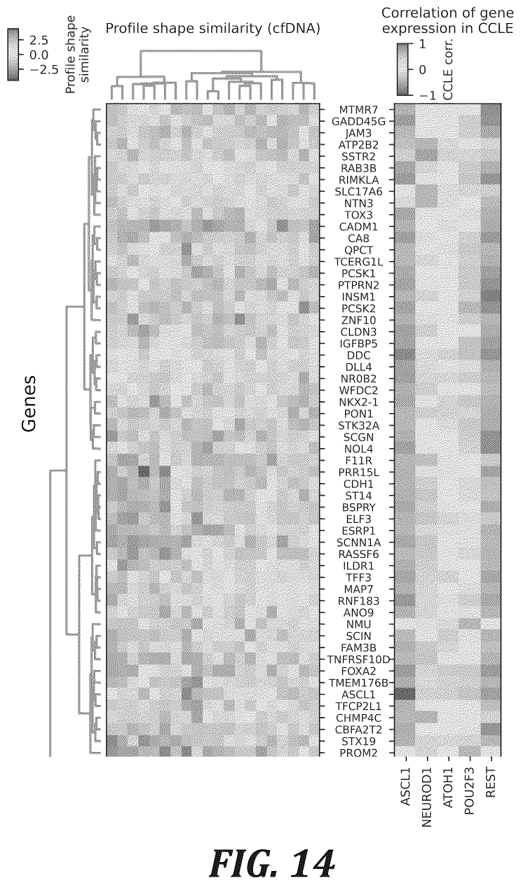

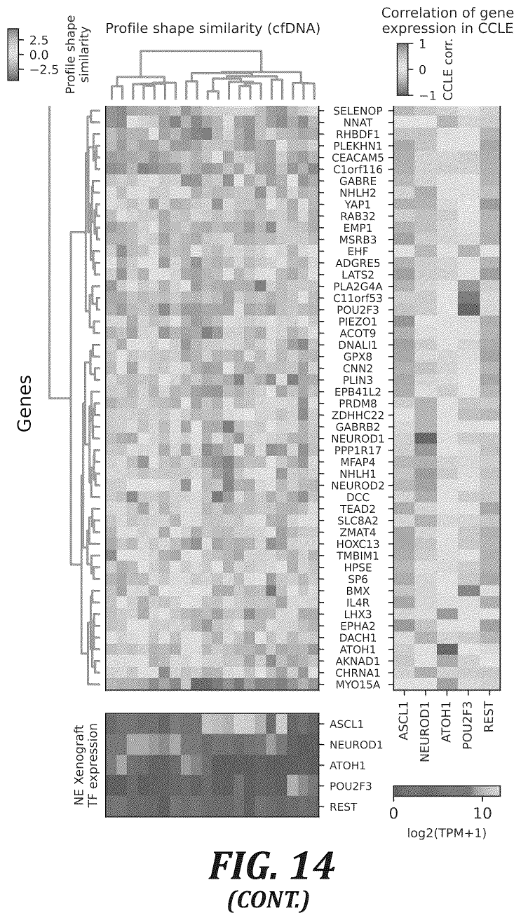

The profile shape similarity metric was also closely examined at individual highly informative TSSs in NE PDX samples, focusing on TSSs of genes that were co expressed with ASCL1, NEUROD1, ATOH1, POU2F3, or REST in SCLC cell line gene expression data (n=106 TSSs) (41) (Methods). Unsupervised clustering of TSS profile shape similarity values grouped NE PDX samples by TF activity (FIG. 14) and grouped TSSs by correlation with SCLC TF expression, suggesting it may be possible to directly interpret individual TSS cfDNA nucleosome profiles (FIG. 14). Notably, genes of these TSSs include TFs that define SCLC subtypes (ASCL1, NEUROD1, POU2F3 and ATOH1), as well as many genes that are functionally linked to these TFs and highly relevant to SCLC biology (46-52). A list curated of 16 TSSs with a high correlation between TSS profile shape similarity and gene expression (Pearson r range 0.64-0.89) was created (FIG. 3G). Included among these were TSSs for ASCL1, NEUROD1, and POU2F3, as well as the ASCL1 target FOXA2, NEUROD1 targets NHLH1 and SSTR2, ATOH1 co-expressed genes LHX3 and MYO15A, and POU2F3 coactivator C11orf53 (recently renamed POU2AF2) (46-52). Hierarchical clustering of the profile shape similarity metric at these 16 TSSs formed four main clusters with distinct SCLC TF activity (FIG. 3H). Altogether these results indicated that the targeted panel includes individual sites that are informative sites for inference of TF activity (TFBSs) or gene expression (TSSs), that informative sites may adopt distinct characteristic profile shapes, and that individual site analysis of selected sites reveals TF activity and single gene expression signals.

Histology Prediction in Patient Samples Using Targeted cfDNA Nucleosome Profiling

Histologic classification is a critical component of lung cancer clinical management at the time of diagnosis. Additionally, reassessment of histologic type at later timepoints is gaining importance as transformation between types, such as LUAD to SCLC in EGFR-mutated NSCLC, has emerged as a key mechanism of resistance to targeted therapies (2, 3, 53). Although histologic classification currently requires tissue sampling, pilot studies have predicted tumor histology using cfDNA (43, 54). It was sought to test whether targeted cfDNA nucleosome profiling could predict lung cancer histologic type, focusing on distinguishing SCLC from NSCLC (both LUAD and LUSC). Although strong composite nucleosome profile signals in PDX samples were observed, admixture of non-tumor cfDNA diminishes signal strength in patient samples (FIG. 10B), making interpretation more challenging (FIG. 10C). Using highly informative sites identified in the previous analysis (TFBSs with adjusted Rand index≥0.5 or TSSs with Mann Whitney U test p<0.01 based on individual site k=2 clustering), a probabilistic framework was formulated analyzing cfDNA nucleosome profiles to simultaneously estimate the tumor fraction and histologic composition of a patient plasma sample (FIG. 4A, FIG. 15A, Methods). Histologic composition was estimated as a continuous value from 0 (NSCLC-like) to 1 (SCLC-like), which we term the “histology score.”

The histology prediction model was trained using PDX samples (n=24, excluding the LCNEC sample) and non-malignant patient samples (n=4) and evaluated the prediction performance using a leave-one-out cross-validation (FIG. 4B). The model predicted uniformly higher tumor fraction for PDX samples (median=0.994, IQR 0.930-1.000) compared to non-malignant patient samples (median=0.053, IQR 0.023 0.109) (two-sided Mann-Whitney U test p=0.0014). Histology scores were also highly concordant with expectation: the SCLC sample median histology score was 1.000 (IQR 0.875-1.000) and the NSCLC sample median histology score was 0.147 (IQR 0.004-0.231) (two-sided Mann-Whitney U test p=5×10⁻⁴) (FIG. 4C). The area under the receiver operating characteristic curve (AUC) was 1.0 for classifying SCLC and NSCLC (FIG. 4D-4E). For MSK_LX1042, an SCLC PDX model from a patient with EGFR-mutated NSCLC that had transformed to SCLC after targeted therapy exposure, the model predicted a histology score of 0.69, consistent with SCLC-like chromatin organization.

After retraining the model on the full training set of PDX samples, it was applied to analyze nine NSCLC and 70 SCLC patient plasma samples with a tumor fraction of 0.05 or greater (FIG. 4F). As expected, the tumor fraction was considerably higher in SCLC patients (median=0.511, IQR 0.360-0.614) relative to NSCLC patients (median=0.241, IQR 0.206-0.276) (two-sided Mann-Whitney U test p=0.0004) (FIG. 4G). Critically, a marked difference in the histology score was also observed between SCLC (median=0.915, IQR 0.830-1.000) and NSCLC (median=0.000, IQR 0.000-0.049) patient samples (two-sided Mann-Whitney U test p=1e-6) (FIG. 4H), with an AUC of 0.994 for classifying NSCLC and SCLC histology (FIG. 4I). Using an optimal histology score threshold of 0.49 determined from the training data (Methods), model sensitivity for SCLC histology was 0.97 and specificity was 0.89. In 27 samples with a tumor fraction less than 0.05 (14 SCLC, 13 NSCLC), the model AUC was 0.88 (FIG. 4H), supporting the use of this approach in earlier stage disease. Together, these results illustrated the use of targeted cfDNA nucleosome profiling for non-invasive discrimination of NSCLC from SCLC.

### SCLC Subtype Inference in Patient Samples Using TF Activity Prediction

Next, the probabilistic framework was updated to predict activity of the key SCLC TFs: ASCL1, NEUROD1, ATOH1, POU2F3, and REST. For each TF, a prediction model was formulated that jointly estimated cfDNA tumor fraction and a “TF score,” ranging from 0 (TF-negative) to 1 (TF-positive) (Methods). For a given TF, model features were restricted to its informative TFBSs (range 0-111, none were used for ATOH1) and informative TSSs (range 7-56) of genes with expression correlated to the TF based on RNAseq of SCLC cell lines (41) (FIG. 15A, Methods).

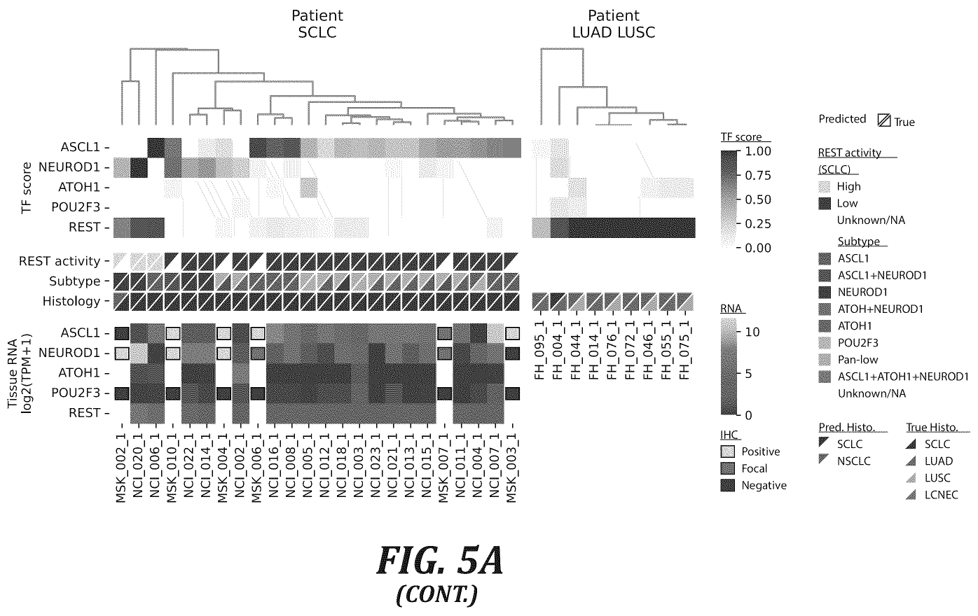

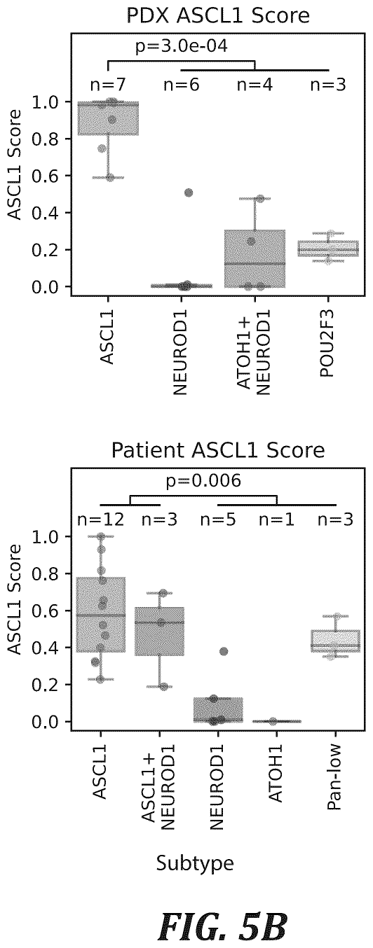

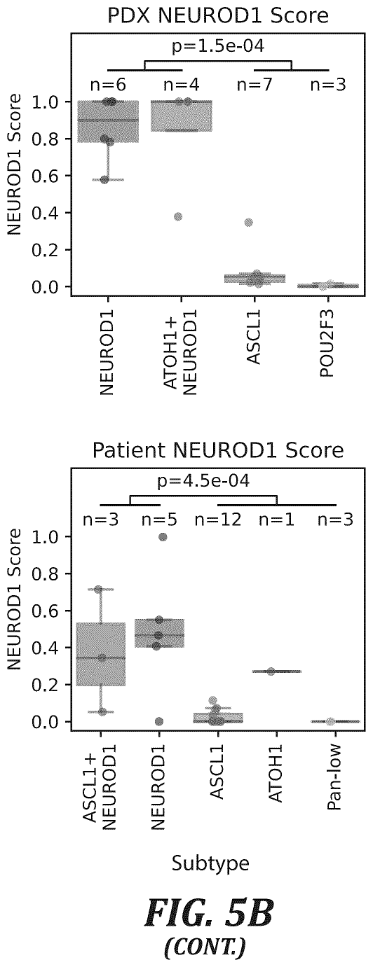

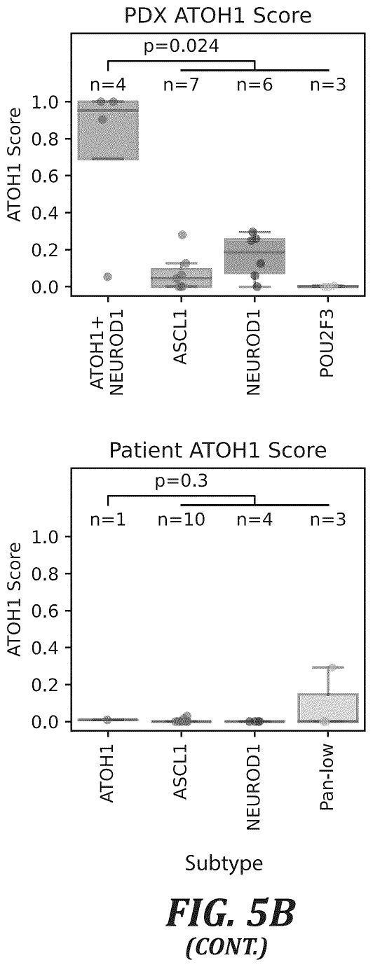

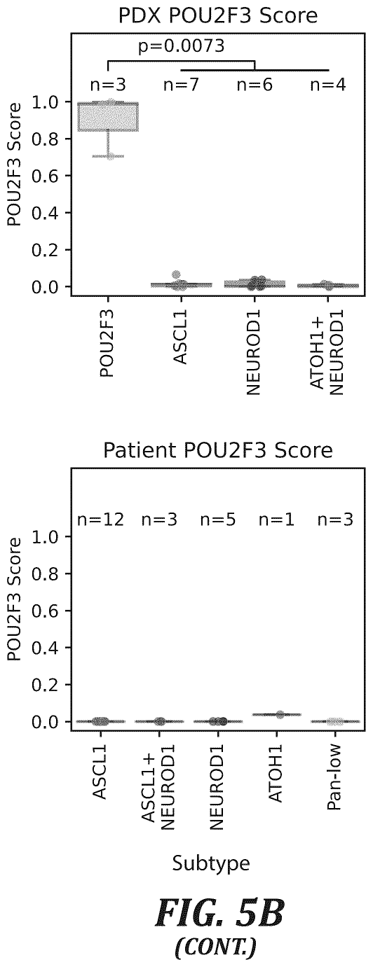

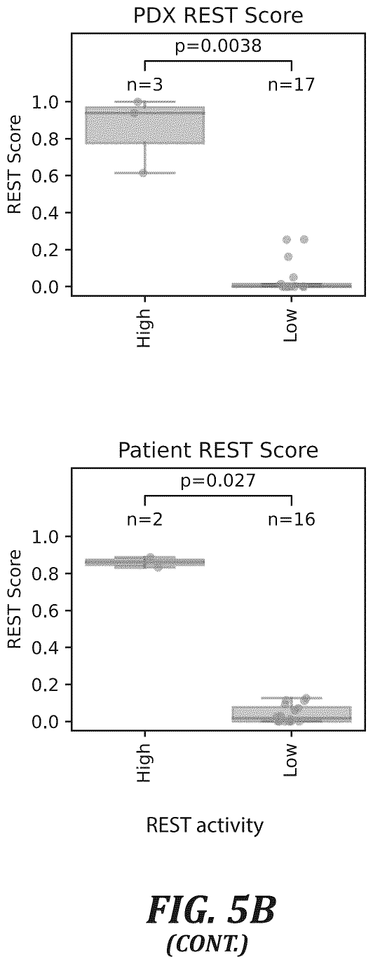

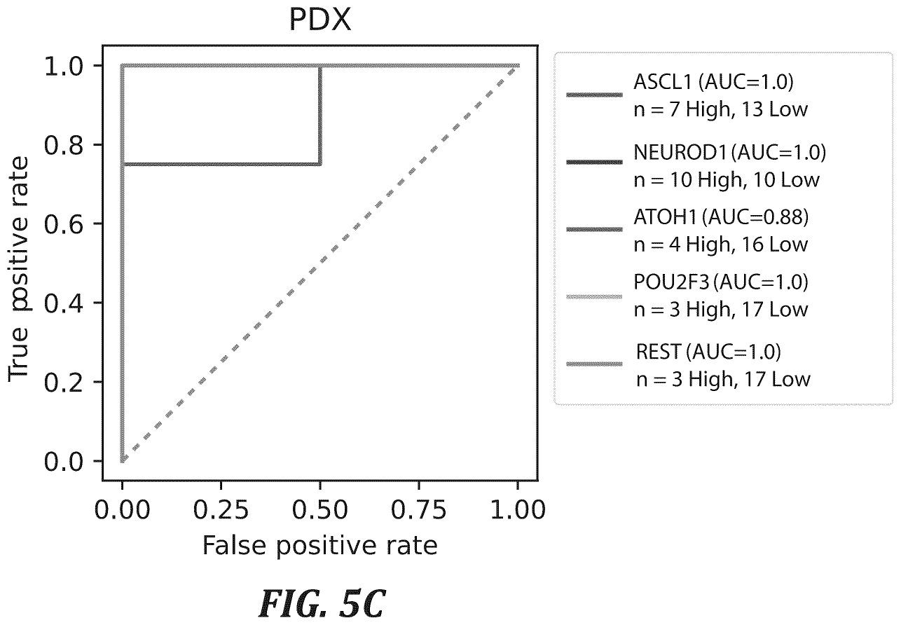

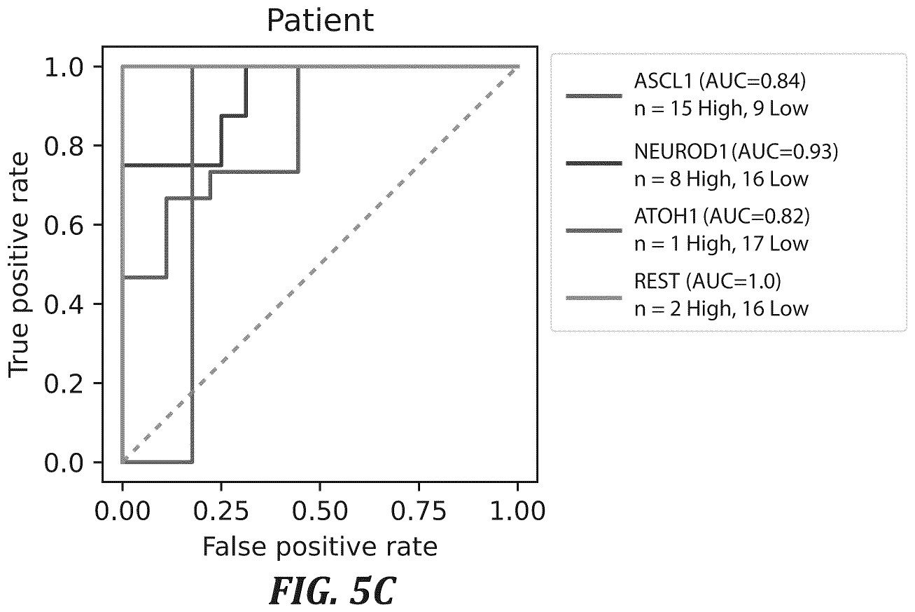

The TF activity prediction models were trained using cfDNA from NE PDX (n=20) and non-malignant patient (n=4) samples and evaluated model performance using leave-one-out cross-validation (FIG. 5A, Methods). The models account for the tumor fraction (i.e., ctDNA fraction) as patient plasma samples contain cfDNA originating from non-tumor cells, largely hematopoietic cells. The tumor fraction was confirmed by performing ultra-low-pass (ULP) WGS and comparing to estimates made by the ichorDNA tool. Using cross-validation, higher TF activity scores were observed for PDX samples categorized by expression of the same factors by RNAseq or immunohistochemistry. It was observed that TF scores were consistent with TF activity in NE PDX samples (FIG. 5B). For example, ASCL1 TF scores were significantly higher in ASCL1-positive PDX samples (median=0.980, IQR 0.825-0.996, n=7) versus ASCL1-negative samples (median=0.01, IQR 0.000-0.244, n=13) (two-sided Mann-Whitney U test p=3×10⁻⁴). Similarly, for NEUROD1, ATOH1, POU2F3, and REST, significantly higher TF scores were observed for PDX samples that expressed these factors (two-sided Mann-Whitney U test p<0.02) (FIG. 5B). As a control, it was noted that the models generated uniformly low ASCL1, NEUROD1, ATOH1, and POU2F3 TF scores in NSCLC PDX samples (median range 0-0.033) and high REST TF scores (median=0.884, IQR 0.815-0.959), consistent with activity patterns of these factors (FIG. 5A). Finally, the models were applied to predict TF activity in the PDX samples and achieved an AUC of 1.0 for each of ASCL1, NEUROD1, POU2F3, and REST, and an AUC of 0.875 for ATOH1 (FIG. 5C).

The models were then retrained on the full training set (NE PDX n=20, non-malignant patient n=4) and applied each model to predict TF scores in SCLC patient plasma samples with available tissue TF activity data (n=24) (FIG. 5A). For the ASCL1, NEUROD1, and REST, the model predicted significantly higher TF scores in patients that were positive for the TF of interest compared to patients not expressing the TF (two-sided Mann-Whitney U test p<0.027) (FIG. 5B). The classification performance of the models in these patient samples by TF activity was 0.84 and 0.88 AUCs for ASCL1 and NEUROD1, respectively (FIG. 5C second panel. The optimal sensitivity was 67% (10/15) at 89% (8/9) specificity for ASCL1 and 75% (6/8) sensitivity at 100% specificity (16/16) for NEUROD1. Using TF score thresholds derived from the training data to classify samples as positive or negative for ASCL1, NEUROD1, or REST, models achieved high specificity (range 0.89-1) and more variable sensitivity (range 0.47-1) (FIG. 15A, Methods). POU2F3 model TF scores were uniformly low (median and IQR 0.0) in the patient cohort, which did not contain any POU2F3-positive tumors (FIGS. 5A-5B). In contrast to the other TFs, it was observed that the ATOH1 TF score (0.0090) was low for the lone patient with an ATOH1-positive tumor despite expected scores for the ATOH1-negative samples (median and IQR=0) (two-sided Mann-Whitney U test p=0.3) (FIG. 5B), although model AUC remained relatively high (0.82) (FIG. 5C). Unlike the four ATOH1 positive PDX models in the training set, the single ATOH1-positive patient sample did not co-express NEUROD1, which may explain the model performance.

To assess an initial baseline of detection for future development/improvements, pairwise admixtures were generated by combining sequencing reads from each of the 19 PDX models with a non-malignant patient sample (n=4) for a total of 96 admixture pairs. For each pair, ctDNA fractions were generated ranging from 0.01 to 0.30 (1,120 admixtures). The TF prediction models bench-marked at AUC>0.7 at 0.10 tumor fraction (FIG. 5C first graph). In silico mixtures were also generated using patient plasma samples and bench-marked at AUC>0.65 (FIG. 5C third graph). Since SCLC has higher disease burden, as 68/84 (81%) of the samples had >0.10 ctDNA fraction (median 0.32), 0.10 ctDNA fraction is a reasonable limit for SCLC prediction.

When applying the models to 9 NSCLC and 46 SCLC patient samples without matched tissue (FIG. 5A, FIG. 15B) and comparing SCLC to NSCLC, higher TF scores were observed for ASCL1 (two-sided Mann-Whitney U test p=8×10⁻⁵) and NEUROD1 (p=0.002) and lower TF scores for REST (p=3×10⁻⁶) (FIG. 15C), consistent with established histology-specific patterns (1, 41). Using thresholds to classify SCLC sample TF activity, the relative frequency of TF activity was consistent with previous reports, such that ASCL1 positivity was the most common (48%), followed by NEUROD1 (20%) and then the other TFs (FIG. 15D) (21, 23, 24). The relative magnitude of ASCL1 and NEUROD1 TF scores was also examined within individual samples. While a substantial fraction of SCLC tumors express both ASCL1 and NEUROD1 transcript or protein (21, 23, 24), it has recently been reported that most tumors are homogeneously composed of single cells expressing an ASCL1- or NEUROD1-specific signature (55). Consistent with this finding, most samples exhibited relatively dominant ASCL1 or NEUROD1 TF scores (FIGS. 15E-15F). Interestingly, two patient samples with matched tissue were ASCL1 and NEUROD1-positive by IHC and both exhibited similar ASCL1 and NEUROD1 TF scores (FIG. 5A, FIGS. 15F-15G), suggesting that our approach can also detect cases of coordinate TF activity. Overall, these results establish that the probabilistic models tailored to target cfDNA nucleosome profiling of informative TFBSs and TSSs reliably predict TF activity to enable subtype classification of lung cancer patient samples.

In the instant disclosure, SCLCpheno-seq was developed, a method that predicts tumor TF activity to infer SCLC subtype and distinguishes SCLC from NSCLC histology using targeted sequencing of native plasma cfDNA. It was hypothesized that targeted nucleosome profiling at TFBSs corresponding to key SCLC TFs and TSSs at transcriptome scale would detect signals of tissue chromatin organization that are characteristic of TF or other gene expression. In this disclosure it was first established that targeted nucleosome profiling readily detected signals of TF and gene expression using a panel of PDX models, and that nucleosome profiles observed in targeted sequencing were congruent with WGS of the same sample. It was also observed that a subset of genomic loci was especially informative as reporters of TF or gene expression, and that some informative sites exhibited nucleosome profiles that diverged widely from the meta-profile observed upon averaging sites together. By independently analyzing individual TSSs, detected signals of single gene expression were detected at highly biologically relevant genes such as ASCL1, ATOH1, NEUROD1, POU2F3, and C11orf53/POU2AF2. Finally, these insights were leveraged to develop prediction models for histology (NSCLC vs SCLC) and SCLC TF activity that were accurate when applied to patient samples. SCLCpheno-seq was also integrated with a genotyping panel to detect mutations in hundreds of cancer-related genes, illustrating its modularity.

This integrated approach simultaneously detected mutations, amplifications and deletions in key disease-related genes while inferring tumor TF activity and gene expression, permitting exploration of candidate genotype-phenotype associations. For example, previous reports have linked MYC activation and NEUROD1 subtype (9, 56). Of the three samples (n=2 patients) in the present disclosure with MYC amplification, all exhibited elevated predicted NEUROD1 activity (TF score range 0.31-0.48). These results highlight the use of this approach to characterize genotype-phenotype links across the natural history of SCLC.

SCLCpheno-seq discriminates SCLC from NSCLC and infers SCLC transcriptional subtype based on TF activity, adding to emerging reports of cfDNA-based subtyping in other tumor types (34, 43, 57). However, the present data also demonstrated the use of targeted cfDNA nucleosome profiling of TSSs to predict the expression of individual disease-relevant genes at scale, which to date has only been reported for a small number of individual genes and applied to cancer detection (35). For example, informative nucleosome profile patterns identified at TSSs of key markers of SCLC subtype including ASCL1, ATOH1, NEUROD1, and POU2F3. Importantly, it was also observed that coordinate TSS profile changes at SCLC TF-associated genes with strong biological connections (FIGS. 3G and 3H). ASCL1 directly binds regulatory regions of the neuroendocrine regulatory TFs INSM1 and FOXA2 (46), and NEUROD1 is associated with increased expression of neuronal axonogenesis gene programs (46, 55) and directly targets NHLH1 (51) and SSTR2 (52). ATOH1 (49), LHX3 (58), and MYO15A (50) are all key co-expressed regulators of auditory hair cell development, while POU2F3 and C11orf53/POU2AF2 are coordinately required during differentiation of chemosensory tuft cells (47, 48). These results support the reliability of targeted nucleosome profiling and suggest that gene expression can be predicted from individual TSSs, dramatically increasing the number of transcriptional features that could be linked to treatment response and resistance. Analysis of larger sample sets will determine the full potential of this approach.

In the work described herein, published SCLC-related ChIPseq and gene expression data (41, 46, 59) was used to select genomic sites for targeted nucleosome profiling. When analyzed in aggregate, these sites yielded signals of TF and gene expression, suggesting that the site selection was generally successful in identifying informative loci. However, in contrast to recent nucleosome profiling efforts, nucleosome profiles at thousands of individual sites were carefully examined. Surprisingly, a minority of the targeted sites were found to be informative with respect to TF or gene expression, and that the observed signal at informative sites sometimes diverged widely from the composite signal obtained by averaging across sites. Heterogeneity in nucleosome profiling site informativeness and structure has not been previously described and has critical implications for optimal targeted panel design and data interpretation.

A key aspect of the disclosed approach is the modularity, customizability, and scalability of hybridization capture-based sequencing. The first iteration (SCLCpheno-seq v1.0) described here targeted 13.2 Mb which enabled broad characterization of nucleosome profiling patterns and performed mutation detection but reduced the feasibility of obtaining deeper (>500×) coverage at individual sites. Importantly, an informative subset of 277 TFBSs and 508 TSSs were identified spanning 460 Kb. A future iteration of SCLCpheno-seq targeting this smaller footprint could practically be sequenced to much deeper coverage, likely generating more precise nucleosome profiling signals and improving performance of SCLC transcriptional subtyping and NSCLC to SCLC trans-differentiation monitoring. Deeper coverage will also likely advance efforts to predict gene expression using single TSSs, potentially reducing or eliminating the need to obtain tissue for RNAseq as a means of broader mechanistic exploration and biomarker discovery.

Here, SCLCpheno-seq was characterized using a set of 25 SCLC patient samples with a spectrum of subtypes; however, this set included only one ATOH1 positive tumor and no POU2F3-positive tumors, limiting the ability to characterize the performance of those predictive models. It should also be noted that SCLCpheno-seq v1.0 was designed prior to the nomination of ATOH1 as a subtype defining gene; inclusion of ATOH1 TFBSs in future iterations improved ATOH1 activity prediction.

Recently, other methods for SCLC TF activity inference using circulating analytes have been described, including DNA methylation profiling (60) and immunoprecipitation of modified nucleosomes in cfDNA (61). Decreased stability of the key analytes (methylated DNA or modified histones) as well as the need for more complex library construction protocols could limit the performance of these methods, especially at lower tumor fraction. In contrast, the disclosed approach is based on targeted sequencing of native cfDNA and is therefore readily integrated into existing high throughput cfDNA sequencing workflows and likely more tolerant of variation in sample collection and storage. For example, SCLCpheno-seq was successfully applied to plasma samples collected up to 12 years prior to DNA isolation in this work. The flexibility of the disclosed approach was also leveraged to individually predict expression of five factors that all play key roles in SCLC biology (ASCL1, NEUROD1, ATOH1, POU2F3, and REST), while other methods have typically focused on fewer factors (54, 60).

In summary, SCLCpheno-seq uses targeted cfDNA nucleosome profiling and detailed knowledge of heterogeneity between individual genomic sites to perform accurate SCLC subtype inference and discrimination of NSCLC vs SCLC histology. This method addresses key gaps in lung cancer care and research. For example, the monitoring of patients with driver mutation positive NSCLC for the development of SCLC trans-differentiation can facilitate more timely change in treatment and improved outcomes. In SCLC patients, SCLCpheno-seq can be used to link transcriptional subtype and genetic mutations to the efficacy of standard and investigational therapies, to explore whether a subtype is plastic over time in individual patients, and to determine patient eligibility for clinical trials that are subtype specific. It is therefore expected that current and future iterations of SCLCpheno-seq can meaningfully advance research and care for patients with lung cancer.

While illustrative embodiments have been illustrated and described, it will be appreciated that various changes can be made therein without departing from the spirit and scope of the invention.

### REFERENCES

1. Rudin C M, Brambilla E, Faivre-Finn C, Sage J. Small-cell lung cancer. Nat. Rev. Dis. Primers. 2021; 7:1-20.
2. Marcoux N, Gettinger S N, O'Kane G, Arbour K C, Neal J W, Husain H, et al. EGFR-Mutant Adenocarcinomas That Transform to Small-Cell Lung Cancer and Other Neuroendocrine Carcinomas: Clinical Outcomes. J. Clin. Oncol. 2019; 37:278-285.
3. Quintanal-Villalonga Á, Chan J M, Yu H A, Pe'er D, Sawyers C L, Sen T, et al. Lineage plasticity in cancer: a shared pathway of therapeutic resistance. Nat. Rev. Clin. Oncol. 2020; 17:360-371.
4. Wistuba I I, Gazdar A F, Minna J D. Molecular genetics of small cell lung carcinoma. Semin. Oncol. 2001; 28:3-13.
5. Mori N, Yokota J, Akiyama T, Sameshima Y, Okamoto A, Mizoguchi H, et al. Variable mutations of the RB gene in small-cell lung carcinoma. Oncogene 1990; 5:1713-1717.
6. George J, Lim J S, Jang S J, Cun Y, Ozretić L, Kong G, et al. Comprehensive genomic profiles of small cell lung cancer. Nature 2015; 524:47-53.
7. Rudin C M, Durinck S, Stawiski E W, Poirier J T, Modrusan Z, Shames D S, et al. Comprehensive genomic analysis identifies SOX2 as a frequently amplified gene in small-cell lung cancer. Nat. Genet. 2012; 44:1111-1116.
8. Augert A, Zhang Q, Bates B, Cui M, Wang X, Wildey G, et al. Small Cell Lung Cancer Exhibits Frequent Inactivating Mutations in the Histone Methyltransferase KMT2D/MLL2: CALGB 151111 (Alliance). J. Thorac. Oncol. 2017; 12:704-713.
9. Mollaoglu G, Guthrie M R, Bohm S, Brägelmann J, Can I, Ballieu P M, et al. MYC Drives Progression of Small Cell Lung Cancer to a Variant Neuroendocrine Subtype with Vulnerability to Aurora Kinase Inhibition. Cancer Cell. 2017; 31:270-285.
10. Rudin C M, Poirier J T, Byers L A, Dive C, Dowlati A, George J, et al. Molecular subtypes of small cell lung cancer: a synthesis of human and mouse model data. Nat. Rev. Cancer 2019; 19:289-97.
11. Gay C M, Stewart C A, Park E M, Diao L, Groves S M, Heeke S, et al. Patterns of transcription factor programs and immune pathway activation define four major subtypes of SCLC with distinct therapeutic vulnerabilities. Cancer Cell. 2021; 39:346-360.e7.
12. Tlemsani C, Pongor L, Elloumi F, Girard L, Huffman K E, Roper N, et al. SCLC-CellMiner: A Resource for Small Cell Lung Cancer Cell Line Genomics and Pharmacology Based on Genomic Signatures. Cell Rep. 2020; 33:108296.
13. Simpson K L, Stoney R, Frese K K, Simms N, Rowe W, Pearce S P, et al. A biobank of small cell lung cancer CDX models elucidates inter- and intratumoral phenotypic heterogeneity. Nat. Cancer. 2020; 1:437-451.
14. Gazdar A F, Carney D N, Nau M M, Minna J D. Characterization of variant subclasses of cell lines derived from small cell lung cancer having distinctive biochemical, morphological, and growth properties. Cancer Res. 1985; 45:2924-2930.
15. Carney D N, Gazdar A F, Bepler G, Guccion J G, Marangos P J, Moody T W, et al. Establishment and identification of small cell lung cancer cell lines having classic and variant features. Cancer Res. 1985; 45:2913-2923.
16. Zhang W, Girard L, Zhang Y-A, Haruki T, Papari-Zareei M, Stastny V, et al. Small cell lung cancer tumors and preclinical models display heterogeneity of neuroendocrine phenotypes. Transl. Lung Cancer Res. 2018; 7:32-49.
17. Cai L, Liu H, Huang F, Fujimoto J, Girard L, Chen J, et al. Cell-autonomous immune gene expression is repressed in pulmonary neuroendocrine cells and small cell lung cancer. Commun. Biol. 2021; 4:314.
18. Lissa D, Takahashi N, Desai P, Manukyan I, Schultz C W, Rajapakse V, et al. Heterogeneity of neuroendocrine transcriptional states in metastatic small cell lung cancers and patient-derived models. Nat. Commun. 2022; 13:2023.
19. Lim J S, Ibaseta A, Fischer M M, Cancilla B, O'Young G, Cristea S, et al. Intratumoural heterogeneity generated by Notch signalling promotes small-cell lung cancer. Nature 2017; 545:360-364.
20. Hong D, Knelson E H, Li Y, Durmaz Y T, Gao W, Walton E, et al. Plasticity in the Absence of NOTCH Uncovers a RUNX2-Dependent Pathway in Small Cell Lung Cancer. Cancer Res. 2022; 82:248-263.
21. Baine M K, Hsieh M-S, Lai W V, Egger J V, Jungbluth A A, Daneshbod Y, et al. SCLC Subtypes Defined by ASCL1, NEUROD1, POU2F3, and YAP1: A Comprehensive Immunohistochemical and Histopathologic Characterization. J. Thorac. Oncol. 2020; 15:1823-1835.
22. Baine M K, Febres-Aldana C A, Chang J C, Jungbluth A A, Sethi S, Antonescu C R, et al. POU2F3 in SCLC: Clinicopathologic and Genomic Analysis With a Focus on Its Diagnostic Utility in Neuroendocrine-Low SCLC. J. Thorac. Oncol. 2022; 17:1109-1121.
23. Megyesfalvi Z, Barany N, Lantos A, Valko Z, Pipek O, Lang C, et al. Expression patterns and prognostic relevance of subtype-specific transcription factors in surgically resected small-cell lung cancer: an international multicenter study. J. Pathol. 2022; 257:674-686.
24. Qu S, Fetsch P, Thomas A, Pommier Y, Schrump D S, Miettinen M M, et al. Molecular Subtypes of Primary SCLC Tumors and Their Associations With Neuroendocrine and Therapeutic Markers. J. Thorac. Oncol. 2022; 17:141-153.
25. Caeser R, Egger J V, Chavan S, Socci N D, Jones C B, Kombak F E, et al. Genomic and transcriptomic analysis of a library of small cell lung cancer patient-derived xenografts. Nat. Commun. 2022; 13:2144.
26. Roper N, Velez M J, Chiappori A, Kim Y S, Wei J S, Sindiri S, et al. Notch signaling and efficacy of PD-1/PD-L1 blockade in relapsed small cell lung cancer. Nat. Commun. 2021; 12:3880.
27. Heitzer E, Auinger L, Speicher M R. Cell-Free DNA and Apoptosis: How Dead Cells Inform About the Living. Trends Mol Med. 2020; 26:519-528.
28. Ignatiadis M, Sledge G W, Jeffrey S S. Liquid biopsy enters the clinic—implementation issues and future challenges. Nat. Rev. Clin. Oncol. 2021; 18:297-312.
29. Snyder M W, Kircher M, Hill A J, Daza R M, Shendure J. Cell-free DNA Comprises an In Vivo Nucleosome Footprint that Informs Its Tissues-Of-Origin. Cell 2016; 164:57-68.
30. Ulz P, Thallinger G G, Auer M, Graf R, Kashofer K, Jahn S W, et al. Inferring expressed genes by whole-genome sequencing of plasma DNA. Nat. Genet. 2016; 48:1273-1278.
31. Ulz P, Perakis S, Zhou Q, Moser T, Belic J, Lazzeri I, et al. Inference of transcription factor binding from cell-free DNA enables tumor subtype prediction and early detection. Nat. Commun. 2019; 10:4666.
32. Cristiano S, Leal A, Phallen J, Fiksel J, Adleff V, Bruhm D C, et al. Genome-wide cell-free DNA fragmentation in patients with cancer. Nature 2019; 570:385-389.
33. Doebley A-L, Ko M, Liao H, Cruikshank A E, Santos K, Kikawa C, et al. A framework for clinical cancer subtyping from nucleosome profiling of cell-free DNA. Nat. Commun. 2022; 13:7475.
34. De Sarkar N, Patton R D, Doebley A-L, Hanratty B, Adil M, Kreitzman A J, et al. Nucleosome patterns in circulating tumor DNA reveal transcriptional regulation of advanced prostate cancer phenotypes. Cancer Discov. 2022; CD-22-0692.
35. Zhu G, Guo Y A, Ho D, Poon P, Poh Z W, Wong P M, et al. Tissue-specific cell-free DNA degradation quantifies circulating tumor DNA burden. Nat. Commun. 2021; 12:2229.
36. Rothwell D G, Ayub M, Cook N, Thistlethwaite F, Carter L, Dean E, et al. Utility of ctDNA to support patient selection for early phase clinical trials: the TARGET study. Nat. Med. 2019; 25:738-743.
37. Cerami E, Gao J, Dogrusoz U, Gross B E, Sumer S O, Aksoy B A, et al. The cBio cancer genomics portal: an open platform for exploring multidimensional cancer genomics data. Cancer Discov. 2012; 2:401-404.
38. Gao J, Aksoy B A, Dogrusoz U, Dresdner G, Gross B, Sumer S O, et al. Integrative analysis of complex cancer genomics and clinical profiles using the cBioPortal. Sci. Signal. 2013; 6:pl1.
39. Rao S, Han A L, Zukowski A, Kopin E, Sartorius C A, Kabos P, et al. Transcription factor-nucleosome dynamics from plasma cfDNA identifies ER-driven states in breast cancer. Sci. Adv. 2022; 8:eabm4358.
40. Almodovar K, Jams W T, Meador C B, Zhao Z, York S, Horn L, et al. Longitudinal Cell-Free DNA Analysis in Patients with Small Cell Lung Cancer Reveals Dynamic Insights into Treatment Efficacy and Disease Relapse. J. Thorac. Oncol. 2018; 13:112-123.
41. Ghandi M, Huang F W, Jané-Valbuena J, Kryukov G V, Lo C C, McDonald E R, et al. Next-generation characterization of the Cancer Cell Line Encyclopedia. Nature 2019; 569:503.
42. Ulz P, Perakis S, Zhou Q, Moser T, Belic J, Lazzeri I, et al. Inference of transcription factor binding from cell-free DNA enables tumor subtype prediction and early detection. Nat. Commun. 2019; 10:4666.
43. Esfahani M S, Hamilton E G, Mehrmohamadi M, Nabet B Y, Alig S K, King D A, et al. Inferring gene expression from cell-free DNA fragmentation profiles. Nat. Biotechnol. 2022; 40:585-597.
44. Herberts C, Annala M, Sipola J, Ng S W S, Chen X E, Nurminen A, et al. Deep whole-genome ctDNA chronology of treatment-resistant prostate cancer. Nature 2022; 608:199-208.
45. Kundaje A, Kyriazopoulou-Panagiotopoulou S, Libbrecht M, Smith C L, Raha D, Winters E E, et al. Ubiquitous heterogeneity and asymmetry of the chromatin environment at regulatory elements. Genome Res. 2012; 22:1735-1747.
46. Borromeo M D, Savage T K, Kollipara R K, He M, Augustyn A, Osborne J K, et al. ASCL1 and NEUROD1 Reveal Heterogeneity in Pulmonary Neuroendocrine Tumors and Regulate Distinct Genetic Programs. Cell Reports 2016; 16:1259-1272.
47. Szczepanski A P, Tsuboyama N, Watanabe J, Hashizume R, Zhao Z, Wang L. POU2AF2/C11orf53 functions as a coactivator of POU2F3 by maintaining chromatin accessibility and enhancer activity. Sci. Adv. 2022; 8:eabg2403.
48. Wu X S, He X-Y, Ipsaro J J, Huang Y-H, Preall J B, Ng D, et al. OCA-T1 and OCA-T2 are coactivators of POU2F3 in the tuft cell lineage. Nature 2022; 607: 169-175.
49. Izumikawa M, Minoda R, Kawamoto K, Abrashkin K A, Swiderski D L, Dolan D F, et al. Auditory hair cell replacement and hearing improvement by Atoh1 gene therapy in deaf mammals. Nat. Med. 2005; 11:271-276.
50. Chen J-R, Tang Z-H, Zheng J, Shi H-S, Ding J, Qian X-D, et al. Effects of genetic correction on the differentiation of hair cell-like cells from iPSCs with MYO15A mutation. Cell Death Differ. 2016; 23:1347-1357.
51. Seo S, Lim J-W, Yellajoshyula D, Chang L-W, Kroll K L. Neurogenin and NeuroD direct transcriptional targets and their regulatory enhancers. EMBO J. 2007; 26:5093-5108.
52. Ma N, Puls B, Chen G. Transcriptomic analyses of NeuroD1-mediated astrocyte-to-neuron conversion. Dev. Neurobiol. 2022; 82:375-391.
53. Quintanal-Villalonga A, Taniguchi H, Zhan Y A, Hasan M M, Chavan S S, Meng F, et al. Multiomic Analysis of Lung Tumors Defines Pathways Activated in Neuroendocrine Transformation. Cancer Discov. 2021; 11:3028-3047.
54. Mathios D, Johansen J S, Cristiano S, Medina J E, Phallen J, Larsen K R, et al. Detection and characterization of lung cancer using cell-free DNA fragmentomes. Nat. Commun. 2021; 12:5060.
55. Chan J M, Quintanal-Villalonga Á, Gao V R, Xie Y, Allaj V, Chaudhary 0, et al. Signatures of plasticity, metastasis, and immunosuppression in an atlas of human small cell lung cancer. Cancer Cell 2021; 39:1479-1496.e18.
56. Ireland A S, Micinski A M, Kastner D W, Guo B, Wait S J, Spainhower K B, et al. MYC Drives Temporal Evolution of Small Cell Lung Cancer Subtypes by Reprogramming Neuroendocrine Fate. Cancer Cell 2020; 38:60-78.e12.
57. Doebley A-L, Ko M, Liao H, Cruikshank A E, Santos K, Kikawa C, et al. A framework for clinical cancer subtyping from nucleosome profiling of cell-free DNA. Nat. Commun. 2022; 13:7475.
58. Hume C R, Bratt D L, Oesterle E C. Expression of LHX3 and SOX2 during mouse inner ear development. Gene Expr. Patterns 2007; 7:798-807.
59. Huang Y-H, Klingbeil O, He X-Y, Wu X S, Arun G, Lu B, et al. POU2F3 is a master regulator of a tuft cell-like variant of small cell lung cancer. Genes Dev. 2018; 32:915-928.
60. Chemi F, Pearce S P, Clipson A, Hill S M, Conway A-M, Richardson S A, et al. cfDNA methylome profiling for detection and subtyping of small cell lung cancers. Nat. Cancer 2022; 3:1260-1270.
61. Fialkoff G, Takahashi N, Sharkia I, Gutin J, Pongor L, Rajan A, et al. Subtyping of Small Cell Lung Cancer using plasma cell-free nucleosomes [Internet]. Cancer Biol. 2022 Jun. Available from: http://biorxiv.org/lookup/doi/10.1101/2022.06.24.497386
62. Pozo K, Kollipara R K, Kelenis D P, Rodarte K E, Ullrich M S, Zhang X, et al. ASCL1, NKX2-1, and PROX1 co-regulate subtype-specific genes in small-cell lung cancer. iScience 2021; 24:102953.
63. Zhang X, Coleman I M, Brown L G, True L D, Kollath L, Lucas J M, et al. SRRM4 Expression and the Loss of REST Activity May Promote the Emergence of the Neuroendocrine Phenotype in Castration-Resistant Prostate Cancer. Clin. Cancer Res. 2015; 21:4698-4708.
64. Chen E Y, Tan C M, Kou Y, Duan Q, Wang Z, Meirelles G V, et al. Enrichr: interactive and collaborative HTML5 gene list enrichment analysis tool. BMC Bioinformatics. 2013; 14:128.
65. Yevshin I, Sharipov R, Kolmykov S, Kondrakhin Y, Kolpakov F. GTRD: A database on gene transcription regulation—2019 update. Nucl. Acids Res. 2019; 47:D100-105.
66. Subramanian A, Tamayo P, Mootha V K, Mukherjee S, Ebert B L, Gillette M A, et al. Gene set enrichment analysis: A knowledge-based approach for interpreting genome-wide expression profiles. Proc. Natl. Acad. Sci. USA 2005; 102:15545-15550.
67. Liberzon A, Birger C, Thorvaldsdóttir H, Ghandi M, Mesirov J P, Tamayo P. The Molecular Signatures Database (MSigDB) hallmark gene set collection. Cell Syst. 2015; 1:417-425.
68. Jo S-Y, Kim E, Kim S. Impact of mouse contamination in genomic profiling of patient-derived models and best practice for robust analysis. Genome Biology. Genome Biol. 2019; 20:231.
69. Crowdis J, He M X, Reardon B, Van Allen E M. CoMut: visualizing integrated molecular information with comutation plots. Robinson P, editor. Bioinformatics 2020; 36:4348-4349.
70. Weirauch M T, Yang A, Albu M, Cote A G, Montenegro-Montero A, Drewe P, et al. Determination and inference of eukaryotic transcription factor sequence specificity. Cell 2014; 158:1431-1443.

The embodiments of the invention in which an exclusive property or privilege is claimed are defined as follows:

1. A method of determining a lung cancer type from a sample comprising cell-free DNA isolated from a patient biological sample, the method comprising:
obtaining nucleotide sequence read data generated from the sample comprising cell-free DNA;
performing a computer-implemented method comprising:
receiving, by a computing system, sequence read data, wherein the sequence read data includes a plurality of fragment reads, wherein each fragment read has a fragment length and a GC content indicating a percentage of bases in the fragment read that are G or C;
determining, by the computing system, GC bias values for each fragment read based on the fragment length and the GC content of the fragment read;
generating, by the computing system, a genomic coverage distribution that is adjusted for GC bias using the sequence read data and the GC bias values;
predicting, by the computing system, the cell type based on the genomic coverage distribution; and
determining the lung cancer type based on the prediction provided by the computer system.

2. The method according to claim 1, wherein determining the GC bias value based on the fragment length and the GC content of the fragment read includes:
counting a number of observed reads of each combination of fragment length and GC content to determine GC counts for the sequence read data;
dividing the GC counts by corresponding GC frequencies in a GC frequency matrix to determine a GC bias for each fragment length;
normalizing a mean GC bias for each fragment length to determine rough GC bias values; and
smoothing the rough GC bias values to determine the GC bias values.

3. The method according to claim 2, wherein the lung cancer is determined to be small cell lung cancer (SCLC) or non-small cell lung cancer (NSCLC).

4. The method according to claim 3, wherein determining the SCLC phenotype includes determining expression of one or more genes of interest.

5. The method according to claim 3, wherein the method is performed a plurality of time over time, and wherein the method further comprises detecting a change from NSCLC to SCLC over time.

6. The method according to claim 3, wherein the patient receives a cancer therapy between performances of the method, wherein the method further comprises determining the responsivity of the NSCLC or SCLC to the treatment.

7. The method according to claim 1, wherein the sequence read data is generated from a panel of genomic targets.

8. The method according to claim 7, wherein the panel of genomic targets comprise transcription factor binding sites (TFBSs) of one or more transcription factors associated with SCLC.

9. The method of claim 8, wherein the one or more transcription factors associated with SCLC comprise one or more of ASLC, ATOH1, NEUROD1, POU2F3, REST, and wherein the method comprises determining the nucleosome occupancy of the TFBSs.

10. The method according to claim 8, wherein the TFBSs are identified by ChIP-seq data, and are retained in the panel if they are proximal to a transcription start site of a gene associated with lung cancer.

11. The method according to claim 7, wherein the panel of genomic targets comprise transcription start sites (TSSs) for one or more markers associated with lung cancer, wherein the method comprises determining the nucleosome occupancy of the TSSs.

12. The method according to claim 4, wherein the method further comprises administering an effective treatment to the patient based on the determined cancer subtype.

13. The method according to claim 5, wherein the method further comprises administering an effective treatment to the patient based on the transition of the lung cancer from NSCLC to SCLC.

14. A method for treating a patient with lung cancer comprising:
obtaining nucleotide sequence read data generated from the sample comprising cell-free DNA;
performing a computer-implemented method comprising:
receiving, by a computer system receiving, by a computing system, sequence read data, wherein the sequence read data includes a plurality of fragment reads, wherein each fragment read has a fragment length and a GC content indicating a percentage of bases in the fragment read that are G or C;
determining, by the computing system, GC bias values for each fragment read based on the fragment length and the GC content of the fragment read;
generating, by the computing system, a genomic coverage distribution that is adjusted for GC bias using the sequence read data and the GC bias values; and
predicting, by the computing system, the cell type based on the genomic coverage distribution;
determining the lung cancer type based on the prediction provided by the computer system; and
administering to the patient an effective therapy for the lung cancer type detected.

15. The method according to claim 14, wherein determining the GC bias value based on the fragment length and the GC content of the fragment read includes:
counting a number of observed reads of each combination of fragment length and GC content to determine GC counts for the sequence read data;
dividing the GC counts by corresponding GC frequencies in a GC frequency matrix to determine a GC bias for each fragment length;
normalizing a mean GC bias for each fragment length to determine rough GC bias values; and
smoothing the rough GC bias values to determine the GC bias values.

16. The method according to claim 15, wherein the lung cancer is determined to be small cell lung cancer (SCLC) or non-small cell lung cancer (NSCLC).

17. The method according to claim 16, wherein determining the SCLC phenotype includes determining expression of one or more genes of interest.

18. The method according to claim 16, wherein the method is performed a plurality of time over time, and wherein the method further comprises detecting a change from NSCLC to SCLC over time.

19. The method according to claim 14, wherein the sequence read data is generated from a panel of genomic targets.

20. The method according to claim 19, wherein the panel of genomic targets comprise transcription factor binding sites (TFBSs) of one or more transcription factors associated with SCLC and/or transcription start sites (TSSs) for one or more markers associated with lung cancer.
